(19) United States
(12) Patent Application Publication
(10) Pub. No.: US 2023/0092770 A1
(43) Pub. Date: Mar. 23, 2023

(54) METHODS AND DEVICES FOR PHOTOBIOMODULATION

(71) Applicant: JelikaLite LLC, New York, NY (US)

(72) Inventors: Eugenia Steingold, New York, NY (US); Katya Sverdlov, New York, NY (US); Michael Ashikhmin, New York, NY (US); Ali Mostashari, New York, NY (US); Sam Steingold, New York, NY (US)

(21) Appl. No.: 17/949,997

(22) Filed: Sep. 21, 2022

Related U.S. Application Data
(63) Continuation-in-part of application No. PCT/US2022/020770, filed on Mar. 17, 2022.
Application No. PCT/US2022/020770 is a continuation-in-part of application No. 17/105,313, filed on Nov. 25, 2020.
Application No. 17/105,313 is a continuation-in-part of application No. PCT/US2020/055782, filed on Oct. 15, 2020.
Application No. PCT/US2020/055782 is a continuation-in-part of application No. 29/728,109, filed on Mar. 16, 2020, now Pat. No. D949355.
(60) Provisional application No. 63/303,384, filed on Jan. 26, 2022.
(60) Provisional application No. 63/272,823, filed on Oct. 28, 2021.
(60) Provisional application No. 63/250,703, filed on Sep. 30, 2021.
(60) Provisional application No. 63/162,484, filed on Mar. 17, 2021.
(60) Provisional application No. 63/033,756, filed on Jun. 2, 2020.
(60) Provisional application No. 62/940,788, filed on Nov. 26, 2019.
(60) Provisional application No. 62/915,221, filed on Oct. 15, 2019.

(51) Int. Cl. A61N 5/06

(52) U.S. Cl. CPC A61N 5/0622; A61N 2005/0647; A61N 2005/0626; A61N 2005/0651; A61N 2005/0663; A61N 2005/0659

(57) ABSTRACT

Systems and methods are described for treatment of neurological conditions in which transcranial illumination using infrared, near-infrared and/or red wavelengths of light are delivered into the brain of a patient using a portable head wearable device. Systems and methods are also described to deliver light to patient tissues for photobiomodulation, particularly through the patient's mouth.

### CROSS REFERENCE TO RELATED APPLICATIONS

This application is a continuation-in-part of International Application No. PCT/US2022/020770, filed Mar. 17, 2022, which claims priority to U.S. Provisional Application No. 63/303,384, filed Jan. 26, 2022, and to U.S. Provisional Application No. 63/272,823, filed Oct. 28, 2021, and to U.S. Provisional Application No. 63/250,703, filed Sep. 30, 2021, and to U.S. Provisional Application No. 63/162,484, filed Mar. 17, 2021. International Application No. PCT/US2022/020770 is also a continuation-in-part of U.S. patent application Ser. No. 17/105,313, filed Nov. 25, 2020, which is a continuation-in-part of International Patent Application No. PCT/US2020/055782, filed Oct. 15, 2020, which claims priority to U.S. Provisional Application No. 63/033,756, filed Jun. 2, 2020, U.S. Provisional Application No. 62/940,788, filed Nov. 26, 2019, U.S. Provisional Application No. 62/915,221, filed Oct. 15, 2019, and U.S. Design Application No. 29/728,109, filed Mar. 16, 2020, the entire contents of each of the above-mentioned applications being incorporated herein by reference.

### FIELD OF THE INVENTION

The presently disclosed subject matter relates generally to methods and devices for transcranial illumination for the therapeutic treatment of neurological conditions. Preferred embodiments can include wearable devices that communicate with mobile devices such as web enabled phones and tablets to facilitate system operation and patient data analysis. This can optionally include cross-modal brain stimulation, diagnostic modalities and, more particularly, provide methods and devices for treating children suffering from autism that can optionally utilize simultaneous audio and light stimulation.

### BACKGROUND

Research indicates that in treating many neurological and psychiatric conditions, a strong combinatory effect of two separate types of treatments exists. For example, in the treatment of depression and anxiety, a combination of both medications and cognitive behavioral therapy (or dialectic behavioral therapy) produces stronger effects than either one of those modalities independently.

Furthermore, music therapy and videos games have been used to treat epilepsy patients. Some of the results indicate that listening to specific musical content in combination with pharmacological treatment reduced both the frequencies of epileptic discharges and frequencies of seizures. Similarly, combining video games with pharmacological treatment has also been shown to modulate the brain neuroplasticity and improve age-related neuronal deficits and enhanced cognitive functions in older adults. Therefore, adding two types of different treatments together has been shown to improve the outcome of the overall treatment of neurological and psychiatric conditions across various domains. Overall, when treating psychiatric or neurological disorder, combinatory effects of brain stimulation through various channels is likely to be stronger than unimodal stimulation.

For children diagnosed with Autism Spectrum Disorder (“ASD”), one of the most common challenges they face is learning language. Studies show that children with ASD struggle with acquiring syntax. As a result, they cannot parse sentences, understand speech, and/or acquire or produce new words. In particular, learning language by the age of five (being able to speak full sentences) is critical for future successful integration with neuro-typical community and independent functioning. In addition, language learning may only occur during the sensitive period (REFS), which ends between 5-7 years of age. If a child does not fully learn language during that period, subsequent learning is highly effortful and achieving fluency is unlikely. Furthermore, being able to comprehend and produce language reduces tantrums and improves behavior in individuals with ASD. Therefore, delays in speech development is one of the most critical symptoms that needs to be alleviated.

Another critical symptom that needs to be alleviated in children with ASD is anxiety. General anxiety is frequently quite debilitating in ASD children and it affects, among other things, children's ability to learn and ability to integrate socially. Children with ASD are frequently prescribed medication to reduce their anxiety, but these medications often have unintended side effects and may be not effective.

In the United States, there are over 1.5 MM children currently diagnosed with ASD, and approximately 80,000 new children are diagnosed with ASD annually. Across the world, approximately 1.5 MM-2 MM new children are annually diagnosed with ASD. Autism services cost Americans approximately $250 billion a year, which includes both medical costs (outpatient care, home care, and drugs) and non-medical costs (special education services, residential services, etc.). In addition to outright costs, there are hidden ones, such as emotional stress as well as the time required to figure out and coordinate care. Research indicates that lifelong care costs can be reduced by almost two thirds with proper early intervention. Further research indicates that ASD is often correlated with mitochondrial dysfunction. Mitochondria in brain cells of autistic individuals does not produce enough adenosine triphosphate (“ATP”). The result of mitochondria dysfunction may be especially pronounced in the brain, since it uses 20% of all the energy generated by the human body, which may lead to neuro-developmental disorders, such as ASD. Encouraging research has shown that infrared and red light may activate a child's mitochondria, and therefore increase ATP production. The metabolic pathways impacting neurological function have been studied in significant detail. See, for example, Naviaux, “Metabolic features and regulation of the healing cycle-A new model for chronic disease pathogenesis and treatment”, Mitochondrion. 2019 May; 46:278-297. doi: 10.1016/j.mito.2018.08.001. Epub 2018 Aug. 9. Note also, Mason et al. “Nitric Oxide inhibition of respiration involves both competitive (heme) and noncompetitive (copper) binding to cytochrome c oxidase”, PNAS, vol. 103, no. 3, Jan. 17, 2006, the entire contents of the above two publications being incorporated herein by reference.

Transcranial photobiomodulation (“tPBM”) of the brain with near infrared and red light has been shown to be beneficial for treating various psychiatric and neurological conditions such as anxiety, stroke and traumatic brain injury. Remarkably, autism spectrum disorder may potentially be treated therapeutically with tPBM as several scientists have recently linked the disorder to mitochondria disbalance and tPBM can potentially affect mitochondria by causing it to produce more ATP. Patients treated with tPBM will absorb near infrared light, which can potentially reduce inflammation, increase oxygen flow to the brain and increase production of ATP. However, devices and methods are needed that will enable additional treatment options for various neurological conditions.

One problem with language acquisition is that many children with ASD cannot focus on the language enough to extract syntactic features of words, to parse sentences, and/or to attend to syntactic and semantic clues of speech. Therefore, their word learning may be delayed.

The problem with anxiety is that ASD children frequently get very stressed and do not know how to calm themselves before a particular learning or social situation. As a result, they are unable to participate in regular activities (such as playdates or classes).

Accordingly, there is a need for improved methods and devices providing treatment of neurological disorders and to specifically provide therapies for the treatment of children.

### SUMMARY

Preferred embodiments provide devices and methods in which a head wearable device is configured to be worn by a subject that is operated to deliver illuminating wavelengths of light with sufficient energy that are absorbed by a region of brain tissue during a therapeutic period. Transcranial delivery of illuminating light can be performed with a plurality of light emitting devices mounted to the head wearable device that can also preferably include control and processing circuitry. Therefore, providing brain stimulation with one or more of, or combinations of (i) infrared, near infrared and red light to improve operational states of the brain such as by ATP production in the brain, for example, and (ii) provide additional specific linguistic input(s) to learn syntax will improve language acquisition in ASD children. Therefore, providing brain stimulation with a combination of (i) near infrared and red light to reduce anxiety and (ii) specific meditations written for ASD children will reduce anxiety. Reduced anxiety leads to both improved language learning and better social integration. Providing an audio language program specifically designed for ASD children, may focus the attention of the child on the language, provide the child with the information about linguistic markers, and improve the child's ability to communicate. This is likely to reduce lifelong care costs for affected individuals.

Preferred embodiments can use a plurality of laser diodes or light emitting diodes (LEDs) configured to emit sufficient power through the cranium of a patient to provide a therapeutic dose during a therapeutic period. This plurality of light emitting devices can be mounted to circuit boards situated on a head wearable device. For the treatment of children the spacing between light emitters in each array mounted to the head wearable device can be selected to improve penetration depth through the cranium. As the cranium of a child increases in thickness with age, the parameters of light used to penetrate the cranium will change as a function of age. As attenuation of the illuminating light will increase with age, the frequency of light, power density and spot size of each light emitter can be selectively adjusted as a function of age. The system can automatically set the illumination conditions as a function of age of the patient. The thickness of the cranium of an individual patient can also be quantitatively measured by x-ray scan and entered into the system to set the desired illumination parameters needed to deliver the required power density to the selected region of the brain. The density of the cranium can also change as a function of age and can be quantitatively measured by x-ray bone densitometer to generate further data that can be used to control and adjust the level of radiance applied to different regions of the cranium.

Aspects of the disclosed technology include methods and devices for cross-modal stimulation brain stimulation, which may be used to treat ASD children. Consistent with the disclosed embodiments, the systems and methods of their use may include a wearable device (e.g., a bandana) that includes one or more processors, transceivers, microphones, headphones, LED lights (diodes), or power sources (e.g., batteries). One exemplary method may include positioning the wearable device on the head of a patient (an ASD child). The method may further include transmitting, by the wearable device (e.g., the LED lights), a pre-defined amount of light (e.g., red or near infrared light). The method may also include simultaneously outputting, by the headphones of the wearable device or other device that can be heard or seen by the patient, a linguistic input to the patient, for example. The linguistic input may include transparent syntactic structures that facilitate, for example, learning how to parse sentences. Also, the method may include outputting specific meditations written for ASD children, that may help ease anxiety, and thus allowing ASD children to better learn language and more easily integrate socially. In some examples, the method may further include receiving a response to the linguistic input from the patient, that the one or more processors may analyze to determine the accuracy of the response and/or to generate any follow-up linguistic inputs. Further, in some examples, the frequency and/or type of light outputted by the wearable device may be adjusted based on the response received from the patient. Also, in some examples, the wearable device may be paired to a user device (e.g., via Bluetooth®) that determines and sends the linguistic input(s) to the wearable device or other devices including one or more transducer devices, such as speakers, or display devices that can generate auditory or visual signals/images that can be heard and/or seen by the patient.

The battery powered headset can preferably be configured with an onboard power control device that automatically controls optical power output of the device during a therapeutic session. Therapeutic sessions can have preset operating conditions for each patient, or a class of patients, as described herein whereby a power distribution circuit board can independently control current levels through each of a plurality of light sources at a selected frequency and duty cycle. Preferred embodiments provide closed loop control of each light source such that the emitted light signal remains within 10% of a nominal value, and preferably within 5% of the selected nominal value. Safety features can be implemented in preferred embodiments in which a sensor can be used to monitor a selected operating condition of the head mounted device. For example, if a patient alters the position of or removes the headset form his or her head during a session, the sensor can transmit a signal to the system control which can, depending upon the received signal, switch off the power to the headset and/or record the time of the signal reception, and optionally send a signal to a remote device by wireless or wired connection communicating the change in state of the device. In another example, if light being emitted by one of the light sources exceeds a threshold value, or if an operating temperature of a component in the optoelectrical system exceeds a threshold temperature, this will trigger a shutoff of the light sources and cause a signal to be sent to an external device communicating the change in operating condition and record time and cause of the change. As the length of a therapeutic session may vary from patient to patient, the operating conditions can be selected based on a plurality of preset operating parameters stored in a device memory. A therapeutic session may last for at least 5 minutes for treatment of certain conditions, whereas the session may last at least 10 minutes for a further condition, and may last 15 or more minutes for a further distinct condition. Power levels may vary for each of these different treatment modules and different light sources may be controlled differently during one or more sessions.

The head wearable device can comprise rigid, semi-rigid or flexible substrates on which the light emitters and circuit elements are attached. The flexible substrates can include woven fabrics or polymer fibers, molded plastics or machine printed components assembled into a band that extends around the head of the patient. Circuit boards on which electrical and optical components are mounted and interconnected can be standard rigid form or they can be flexible so as to accommodate bending around the curvature of the patient's head. As children and adults have heads in a range of different sizes, it is advantageous to have a conformable material that can adjust to different sizes. More rigid head wearable devices can use foam material to provide a conformable material in contact with the patient's head. The head wearable device can be used in conjunction with diagnostic devices and systems that can be used to select the parameters for the therapeutic use of light as described herein. A computing device such as a tablet or laptop computer can be used to control diagnostic and therapeutic operations of the head worn device and other devices used in conjunction with a therapeutic session. Such computing devices can store and manage patient data and generate electronic health or medical records for storage and further use. The computing device can be programmed with software modules such as a patient data entry module, a system operating module that can include diagnostic and therapeutic submodules, and an electronic medical records module. The system can include a networked server to enable communication with remote devices, web/internet operations and remote monitoring and control by secure communication links. The computing device can include connections to electroencephalogram (EEG) electrodes to monitor brain activity before, during or after therapeutic sessions to generate diagnostic data for the patient. The EEG electrodes can be integrated with the head wearable device and be connected either directly to a processor thereon, or alternatively, can communicate by wired or wireless connection to the external computing device such as a touchscreen operated tablet display device. Light sensors that are optically coupled to the head of the patient can be used to monitor light delivery into the cranium of the patient and/or can measure light returning from the regions of the brain that receive the illuminating light. An array of near infrared sensors can be mounted on the LED panels or circuit boards, for example, that can detect reflected light or other light signals returning from the tissue that can be used to diagnose a condition of the tissue. Diagnostic data generated by the system sensors can be used to monitor the patient during a therapeutic period and can optionally be used to control operating parameters of the system during the therapy session such as by increasing or decreasing the intensity of the light delivered through the cranium or adjusting the time period or areas of the brain being illuminated during the therapy session.

For the treatment of children having an autism spectrum disorder, they often are not responsive to instructions, may exhibit behaviors such as self-injury or attempt to injure others, and may exhibit movements that are not conducive to standard therapeutic treatment. Specifically, it can be necessary with many patients that a device placed on the head must be light in weight and be untethered such as by a wired connection during treatment. Consequently, it is important to have a battery powered device that does not have a wired connection during a therapeutic period or session. Any communication that occurs between the head mounted device and an external device used to control and/or monitor the device during a therapeutic period is preferably performed by wireless connection. Thus, an external computing device such as a mobile communication device such as a mobile phone or a tablet display device can communicate wirelessly with the head mounted device. Such devices can include one or more processors configured to stream data to and from the head mounted device. Such devices are connectable to private or public communications networks to facilitate communication with parents and teachers, for example, that are involved with a child's treatment, medical history and education plan.

Machine learning tools can be employed to process data generated by the devices and methods described herein. Methods such as principal component analysis (PCA), support vector machines (SVM), convolutional and/or recurrent neural networks, clustering and other numeric and quantitative methods can be employed to characterize therapeutic outcomes and generate operational parameters for different classes of patients that exhibit different behavioral and/or medical conditions that can be effectively treated by photobiomodulation therapy. Neurologic conditions can impact sleep patterns and learning capacity of children and such computational methods can be used to improve therapeutic treatment.

Further features of the disclosed design, and the advantages offered thereby, are explained in greater detail hereinafter with reference to specific embodiments illustrated in the accompanying drawings, wherein like elements are indicated be like reference designators.

### BRIEF DESCRIPTION OF THE DRAWINGS

Reference will now be made to the accompanying drawings, which are not necessarily drawn to scale, are incorporated into and constitute a portion of this disclosure, illustrate various implementations and aspects of the disclosed technology, and, together with the description, serve to explain the principles of the disclosed technology. In the drawings:

FIG. 1 is an example head wearable device, in accordance with some examples of the present disclosure.

FIGS. 2A-2C show rear, side and front views of a patient with the head wearable device of FIG. 1.

FIG. 3 illustrates use of a portable phone or tablet device connected to the head wearable device.

FIG. 4 schematically illustrates the operating elements of the head wearable device and control features.

FIG. 5 schematically illustrates the components of a head wearable device in accordance with preferred embodiments.

FIG. 6 illustrates a screen shot of a testing procedure used with preferred embodiments of the invention.

FIG. 7 illustrates a light emitter for transcranial illumination mounted on one side of a circuit board that is mounted to the head wearable device.

FIG. 8 illustrates a second side of the circuit board shown in FIG. 7 including connectors to the controller and power source for the head wearable device.

FIG. 9 illustrates a detailed view showing circuit board elements of the head wearable device of certain embodiments.

FIG. 10 shows circuitry mounted on a circuit board of the head wearable device for preferred embodiments.

FIG. 11 shows circuitry mounted on a circuit board for control of preferred embodiments of a head wearable device.

FIG. 12 shows circuitry mounted on a circuit board to control operations of the head wearable device.

FIG. 13 is a process flow diagram in accordance with preferred methods of operating the head wearable device and control system.

FIG. 14 is a process flow diagram illustrating the use of EEG measurements in conjunction with transcranial illumination of a patient.

FIG. 15 illustrates a table with exemplary parameters having variable ranges between upper and lower thresholds used for transcranial illumination of a patient in accordance with preferred embodiments.

FIG. 16 illustrates a process flow diagram for selecting and optimizing parameters over multiple therapeutic sessions including manual and automated selection tracks.

FIG. 17 illustrates a process flow diagram for administering a therapeutic session to a patient in accordance with various embodiments described herein.

FIG. 18A illustrates a further view of a head worn device having circuit housing elements accessible to a user that is communicably connected to a first tablet device used by the patient during a therapy session and a second tablet used by an operator to monitor, control and/or program the system for diagnostic and therapeutic use as described generally herein.

FIG. 18B illustrates a top view of a head mounted photobiomodulation system including an electroencephalographic (EEG) electrode system with wireless transmission of data to an external processing system.

FIG. 18C shows a rear view of the system of FIG. 18B with light emitting and/or sensor arrays mounted on a rear band configured for positioning to transmit and/or receive signals through the cranium via selected transmission paths such as, for example, the lambdoid suture and/or the squamosal suture.

FIG. 18D illustrates an enlarged side cross-sectional view of a spring mounted light emitting array, a sensor array, or a combination thereof in accordance with embodiments described herein.

FIG. 18E illustrates a side view of a head wearable device in accordance with some embodiments described herein.

FIG. 18F illustrates a rear view of the head wearable device of FIG. 18E.

FIG. 18G illustrates an alternative embodiment of the head wearable device with different placement of the head strap in accordance with some embodiments described herein.

FIG. 18H illustrates a head wearable device according to various embodiments described herein.

FIG. 18I illustrates placement of an LED module in a core of a headband in accordance with some embodiments described herein.

FIG. 18J illustrates placement of the LED module in a completed headband in accordance with some embodiments described herein.

FIG. 18K schematically illustrates the electrical connections between elements of the head wearable device in accordance with several embodiments described herein.

FIGS. 18L and 18M illustrate top and bottom views, respectively, of a power printed circuit board in accordance with some embodiments described herein.

FIGS. 18N and 18O illustrate top and bottom views, respectively, of an occipital power distribution printed circuit board in accordance with some embodiments described herein.

FIGS. 18P and 18Q illustrate top and bottom views, respectively, of a frontal power distribution printed circuit board in accordance with some embodiments described herein.

FIGS. 18R and 18S illustrate top and bottom views, respectively, of an LED printed circuit board in accordance with some embodiments described herein.

FIG. 19 illustrates a process sequence that can be implemented with the therapeutic devices described herein.

FIG. 20 illustrates an exemplary photobiomodulation device in accordance with embodiments described herein.

FIGS. 21A and 21B illustrate side and perspective views, respectively, of a device for partial insertion within an oral cavity in accordance with certain embodiments described herein.

FIGS. 22A and 22B illustrate side and perspective views, respectively, of a device for partial insertion within an oral cavity in accordance with certain embodiments described herein.

FIGS. 23A and 23B illustrate cross-section and end views, respectively, of an exemplary device for partial insertion according to certain embodiments.

FIG. 24 illustrates a circuit for operating photobiomodulation devices of the present description.

FIG. 25 illustrates a battery discharge curve in accordance with some embodiments of the present description.

FIG. 26 shows a plot of relative output flux as a function of forward current in accordance with some embodiments of the present description.

FIG. 27 shows a plot of relative voltage as a function of forward current in accordance with some embodiments of the present description.

FIG. 28 shows a plot of relative output flux as a function of temperature in accordance with some embodiments of the present description.

FIG. 29 shows resting power as a function of frequency bands for patients with different diagnoses.

FIG. 30 is a block diagram representing the user assessment, personalized treatment selection, and performance feedback process.

FIG. 31 is a block diagram for the reference population treatment effectiveness cluster analysis for personalized intervention clusters using machine learning.

FIG. 32 is a block diagram for the machine learning model used in the machine learning module (MLM) to create personalized treatment clusters based on reference population data.

FIG. 33 is an illustration of an exemplary system that may be used to implement the functions and processes of certain embodiments of the present invention.

FIGS. 34A and 34B illustrate delta wave EEG intensities measured for experimental (“active”) and control (“placebo”) groups, respectively, in a clinical trial utilizing photobiomodulation devices and techniques as described herein.

FIGS. 34C, 34D and 34E show an overlay of active and placebo delta wave EEG results after seven sessions of data collection, a cumulative composite score between the active and placebo groups, and a CARS scale test results for active and placebo control groups (before and after), respectively.

FIGS. 35A and 35B illustrate benefit index and side effect index scores, respectively, for experimental and control group subjects in a clinical trial utilizing photobiomodulation devices and techniques as described herein.

FIG. 36 illustrates a method for therapeutic photobiomodulation for treatment of diseases or disorders in accordance with some embodiments described herein.

### DETAILED DESCRIPTION

Some implementations of the disclosed technology will be described more fully with reference to the accompanying drawings. This disclosed technology can be embodied in many different forms, however, and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein can include, but are not limited to, for example, components developed after development of the disclosed technology.

It is also to be understood that the mention of one or more method steps does not imply that the methods steps must be performed in a particular order or preclude the presence of additional method steps or intervening method steps between the steps expressly identified.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

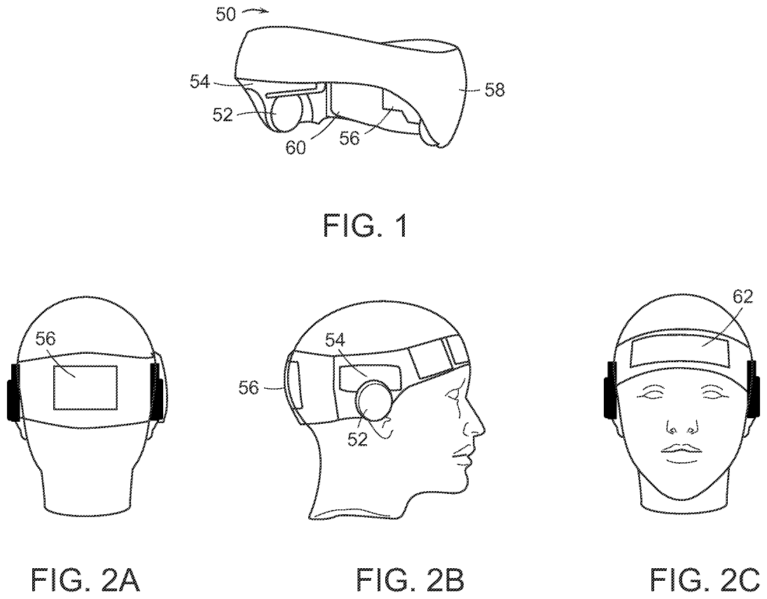

FIG. 1 shows an example wearable device 50 that may implement certain methods for cross-modal brain stimulation. As shown in FIG. 1, in some implementations the wearable device 50 may include one or more processors, transceivers, microphones, headphones 52, LED lights 54, and/or batteries, amongst other things. The wearable device 50 may be paired with a user device (e.g., smartphone, smartwatch), which may provide instructions that may determine a frequency of transmitted light, the type of light (e.g., red light or infrared light), the meditations, and/or the linguistic inputs. FIGS. 2A-2C depict rear side and front views of the head wearable device 50 positioned on the head of a patient with rear circuit board 56, side illumination panels 56, and front illumination panel 62 to provide transcranial illumination, and also earphones 52 to provide audio programming to the patient. The system can store audio files or video files that can be heard or seen by the user in conjunction with the therapeutic session for a patient.

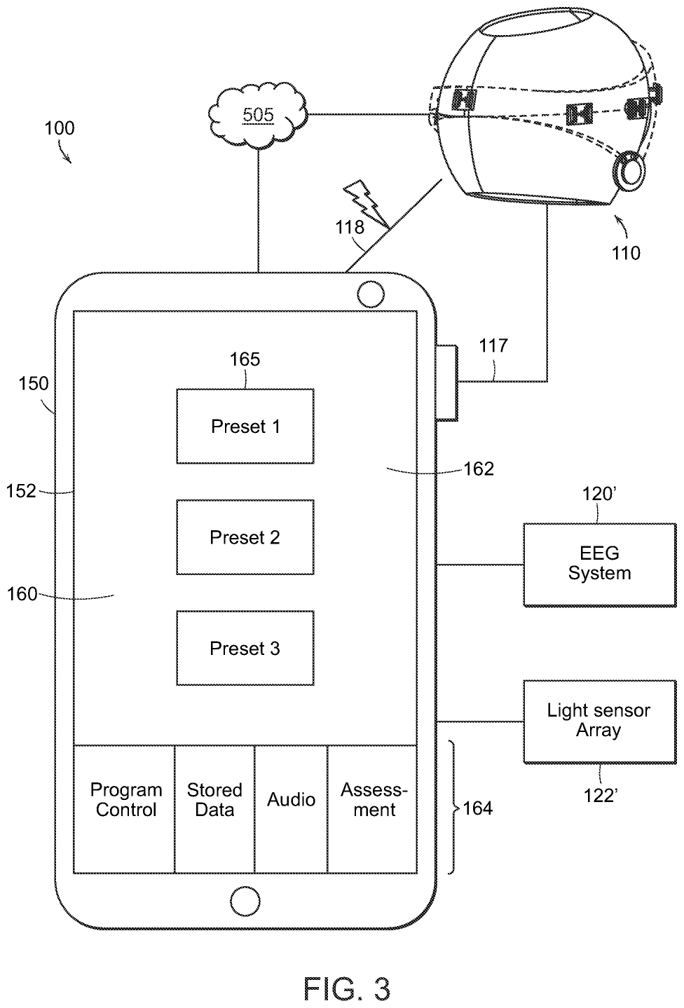

FIG. 3 is an illustration of a system 100 for brain stimulation in accordance with various embodiments described herein. The system 100 includes a photobiomodulation device 110 in communication with a remote computing device 150. In exemplary embodiments, the computing device 150 includes a visual display device 152 that can display a graphical user interface (GUI) 160. The GUI 160 includes an information display area 162 and user-actuatable controls 164. Optionally, the computing device 150 is also in communication with an external EEG system 120′. Optionally, the computing device 150 is also in communication with an external light sensor array 122′. An operating user can operate the computing device 150 to control operation of the photobiomodulation device 110 including activation of the functions of the photobiomodulation device 110 and mono- or bi-directional data transfer between the computing device 150 and the photobiomodulation device 110.

The operating user can change among operational modes of the computing device 150 by interacting with the user-actuatable controls 164 of the GUI 160. Examples of user-actuatable controls include controls to access program control tools, stored data and/or stored data manipulation and visualization tools, audio program tools, assessment tools, and any other suitable control modes or tools known to one of ordinary skill in the art. Upon activation of the program control mode, the GUI 160 displays program control information in the information display area 162. Likewise, activation of other modes using user-actuatable controls 164 can cause the GUI 160 to display relevant mode information in the information display area 162. The system can be programmed to perform therapeutic sessions with variable lengths of between 5 and 30 minutes, for example. The patient's use of language during the session can be recorded by microphone on the head wearable device or used separately and an analysis of language used during the session or stored for later analysis.

In the program control mode, the GUI 160 can display program controls including one or more presets 165. Activation of the preset by the operating user configures the photobiomodulation device 110 to use specific pre-set variables appropriate to light therapy for a particular class of patients or to a specific patient. For example, a specific preset 165 can correspond to a class of patient having a particular age or particular condition. In various embodiments, the pre-set variables that are configured through the preset 165 can include illumination patterns (e.g., spatial patterns, temporal patterns, or both spatial and temporal patterns), illumination wavelengths/frequencies, or illumination power levels.

In some embodiments, the photobiomodulation device 110 can transmit and/or receive data from the computing device 150. For example, the photobiomodulation device 110 can transmit data to log information about a therapy session for a patient. Such data can include, for example, illumination patterns, total length of time, time spent in different phases of a therapy program, electroencephalogram (EEG) readings, and power levels used. The data can be transmitted and logged before, during, and after a therapy session. Similar data can also be received at the computing device 150 from the external EEG system 120′ or the external light sensor array 122′ in embodiments that utilize these components. In the stored data manipulation and/or visualization mode, the operating user can review the data logged from these sources and received at the computing device 150. In some embodiments, the data can include information regarding activities used in conjunction with the therapy session (i.e., information related to tasks presented to the patient during the therapy session such as task identity and scoring). For example, activity data can be input by an operating user on the assessment mode screen as described in greater detail below.

In the audio system mode, the user can control audio information to be delivered to the patient through speakers 116 of the photobiomodulation device 110. Audio information can include instructions to the patient in some embodiments. In other embodiments, audio information can include audio programming for different therapeutic applications.

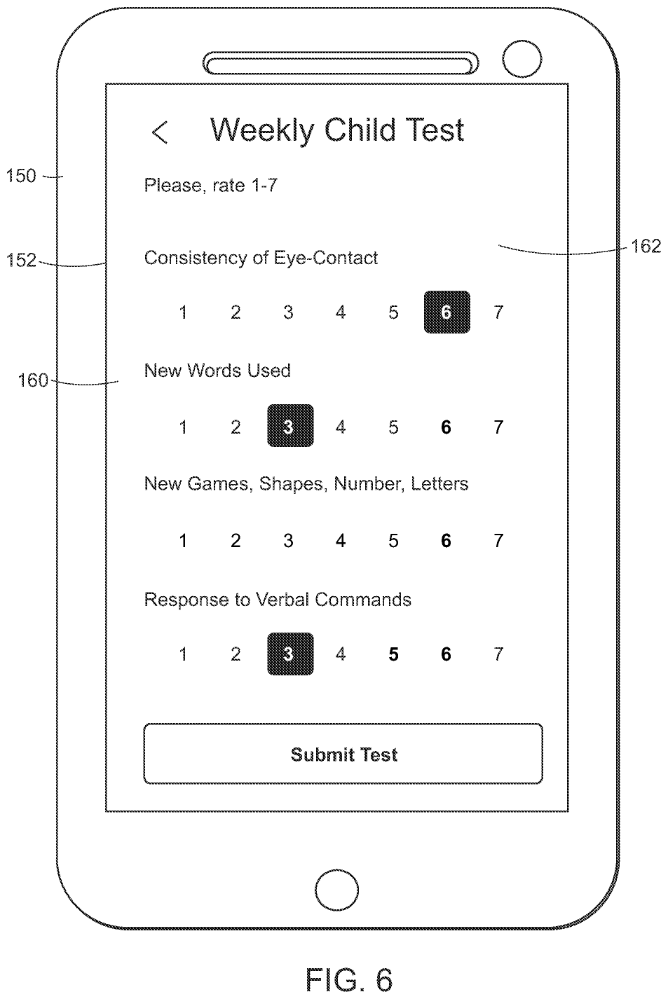

In the assessment mode, a user can input or review data related to patient assessment such as task identity and scoring. For example, FIG. 6 illustrates a particular assessment test displayed in the information display area 162 of the GUI 160. This assessment test, the Weekly Child Test, includes rating scales representing scoring on a variety of individual metrics geared to an overall assessment of the severity of autism in the child.

As described in greater detail below, the computing device 150 and photobiomodulation device 110 can communicate through a variety of methods. In some embodiments, a direct (i.e., wired) connection 117 can be established between the computing device 150 and the photobiomodulation device 110. In some embodiments, the computing device 150 and the photobiomodulation device 110 can communicate directly with one another through a wireless connection 118. In still further embodiments, the computing device 150 and the photobiomodulation device 110 can communication through a communications network 505.

In various embodiments, one or more portions of the communications network 505 can be an ad hoc network, a mesh network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a Wi-Fi network, a WiMAX network, an Internet-of-Things (IoT) network established using Bluetooth® or any other protocol, any other type of network, or a combination of two or more such networks.

In exemplary embodiments, the system 100 is configured to treat autistic patients and, in particular, juvenile autistic patients. As such, it is desirable in many embodiments to create a wireless connection between the photobiomodulation device 110 and the computing device 150 as a juvenile patient is less likely to sit still for the length of a therapy session. Wireless connection and use of a battery to power the photobiomodulation device 110 enables uninterrupted transcranial illumination for the entire length of a single therapy session and, further, enables the juvenile patient to move and engage in activities that may, or may not, be associated with the therapy.

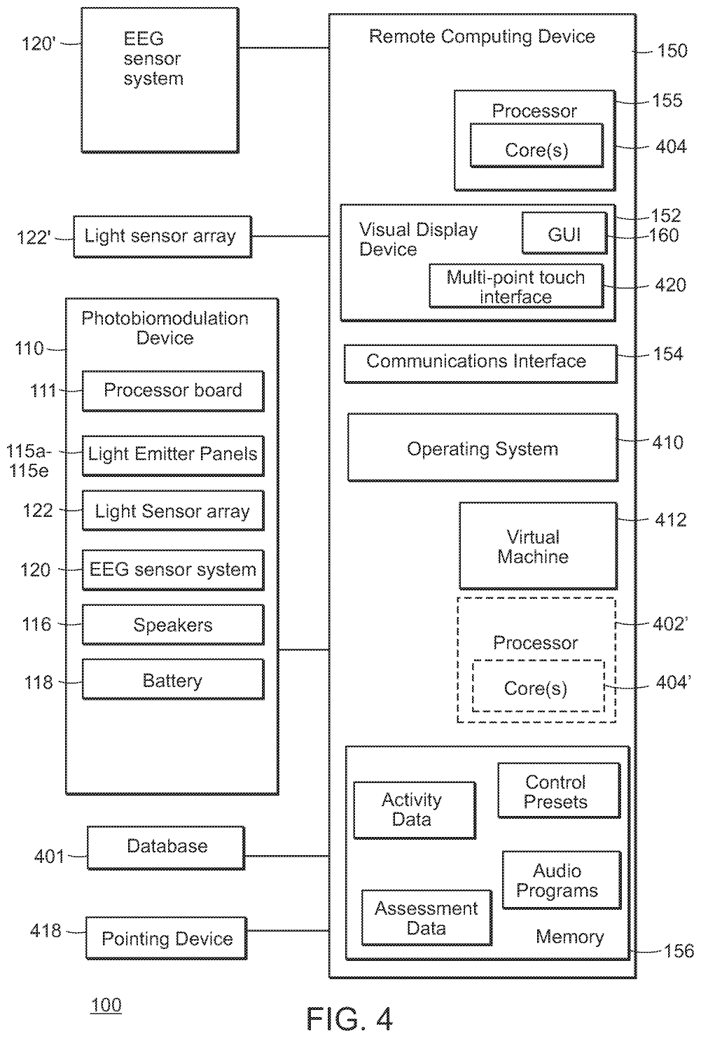

FIG. 4 shows block diagrams of a remote computing device 150 and photobiomodulation device 110 suitable for use with exemplary embodiments of the present disclosure. The remote computing device 150 may be, but is not limited to, a smartphone, laptop, tablet, desktop computer, server, or network appliance. The remote computing device 150 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives, one or more solid state disks), and the like. For example, memory 156 included in the remote computing device 150 may store computer-readable and computer-executable instructions or software for implementing exemplary operations of the remote computing device 150. The remote computing device 150 also includes configurable and/or programmable processor 155 and associated core(s) 404, and optionally, one or more additional configurable and/or programmable processor(s) 402′ and associated core(s) 404′ (for example, in the case of computer systems having multiple processors/cores), for executing computer-readable and computer-executable instructions or software stored in the memory 156 and other programs for implementing exemplary embodiments of the present disclosure. Processor 155 and processor(s) 402′ may each be a single core processor or multiple core (404 and 404′) processor. Either or both of processor 155 and processor(s) 402′ may be configured to execute one or more of the instructions described in connection with remote computing device 150.

Virtualization may be employed in the remote computing device 150 so that infrastructure and resources in the remote computing device 150 may be shared dynamically. A virtual machine 412 may be provided to handle a process running on multiple processors so that the process appears to be using only one computing resource rather than multiple computing resources. Multiple virtual machines may also be used with one processor.

Memory 156 may include a computer system memory or random access memory, such as DRAM, SRAM, EDO RAM, and the like. Memory 156 may include other types of memory as well, or combinations thereof.

A user may interact with the remote computing device 150 through a visual display device 152, such as a computer monitor, which may display one or more graphical user interfaces 160. In exemplary embodiments, the visual display device includes a multi-point touch interface 420 (e.g., touchscreen) that can receive tactile input from an operating user. The operating user may interact with the remote computing device 150 using the multi-point touch interface 420 or a pointing device 418.

The remote computing device 150 may also interact with one or more computer storage devices or databases 401, such as a hard-drive, CD-ROM, or other computer readable media, for storing data and computer-readable instructions and/or software that implement exemplary embodiments of the present disclosure (e.g., applications). For example, exemplary storage device 401 can include modules to execute aspects of the GUI 160 or control presets, audio programs, activity data, or assessment data. The database(s) 401 may be updated manually or automatically at any suitable time to add, delete, and/or update one or more data items in the databases. The remote computing device 150 can send data to or receive data from the database 401 including, for example, patient data, program data, or computer-executable instructions.

The remote computing device 150 can include a communications interface 154 configured to interface via one or more network devices with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections (for example, WiFi or Bluetooth®), controller area network (CAN), or some combination of any or all of the above. In exemplary embodiments, the remote computing device 150 can include one or more antennas to facilitate wireless communication (e.g., via the network interface) between the remote computing device 150 and a network and/or between the remote computing device 150 and the photobiomodulation device 100. The communications interface 154 may include a built-in network adapter, network interface card, PCMCIA network card, card bus network adapter, wireless network adapter, USB network adapter, modem or any other device suitable for interfacing the remote computing device 150 to any type of network capable of communication and performing the operations described herein.

The remote computing device 150 may run operating system 410, such as versions of the Microsoft® Windows® operating systems, different releases of the Unix and Linux operating systems, versions of the MacOS® for Macintosh computers, embedded operating systems, real-time operating systems, open source operating systems, proprietary operating systems, or other operating system capable of running on the remote computing device 150 and performing the operations described herein. In exemplary embodiments, the operating system 410 may be run in native mode or emulated mode. In an exemplary embodiment, the operating system 410 may be run on one or more cloud machine instances.

The photobiomodulation device 110 can include a processor board 111, one or more light emitter panels 115a-115e, one or more speakers 116, and one or more batteries 118. The photobiomodulation device 110 can optionally include a light sensor array 122 and an EEG sensor system 120. Although five light emitter panels 115a-115e are described with respect to this disclosure, one or ordinary skill in the art would appreciate that a greater or fewer number of panels may be used. In an exemplary embodiment, the light emitter panels 115a-115e hare flexible. In an exemplary embodiment, the light emitter panels 115a-115e are positioned at the front, top, back, and both sides of the user's head. In embodiments wherein the photobiomodulation device 110 does not have a full cap over the user's head (i.e., a headband-style device), the top panel may be omitted.

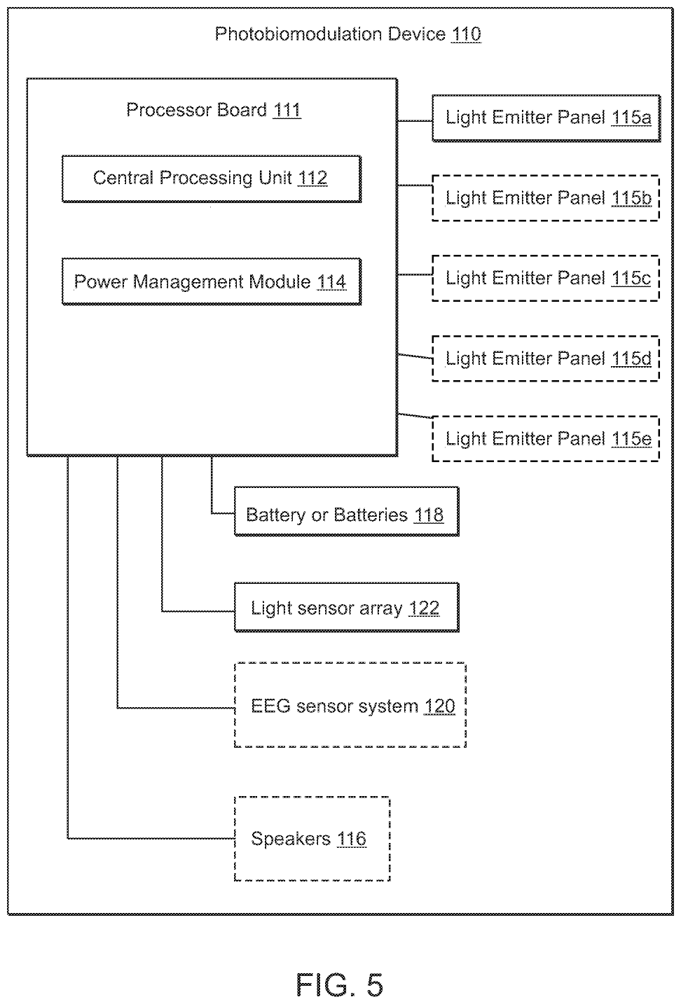

FIG. 5 illustrates a schematic layout of the photobiomodulation device 110 of the present invention. The processor board 11I is, for example, a printed circuit board including components to control functions of the photobiomodulation device 110. The processor board 111 can include a central processing unit 112 and a power management module 114 in some embodiments.

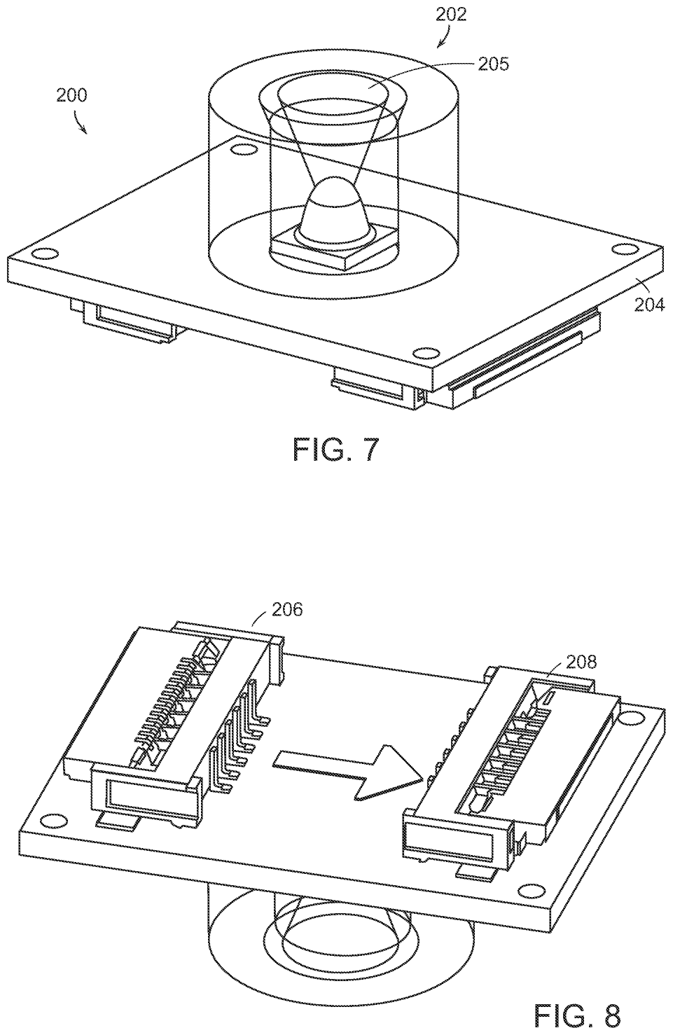

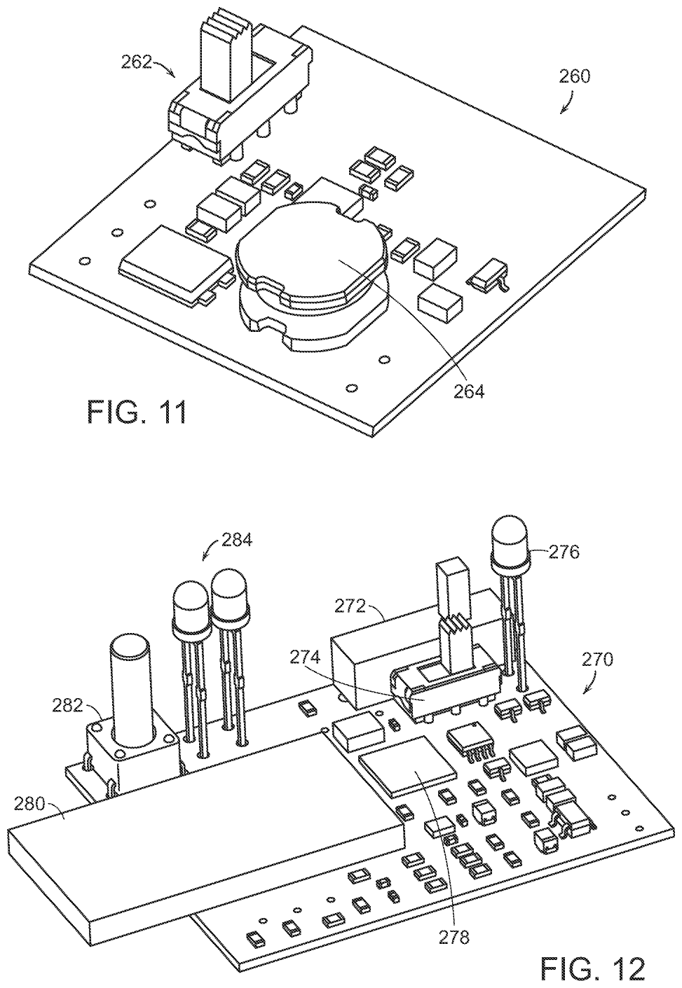

The power management module 114 can monitor and control use of particular light emitter panels 115a-115e during a therapy session. In some embodiments, the power management module 114 can take action to control or provide feedback to a patient user related to whether light emitter panels 115a- 115e are not used, or are only partially used, during a particular therapy session. By mitigating use of certain panels during a session, longer operation can be achieved. Moreover, different classes of patient (e.g., patients of different ages) can have different cranial thicknesses. As a result, different transmission power (and penetration) may be necessary as a function of patient age. The power management module 114 can control power output to light emitter panels to provide a therapeutically beneficial dose of illumination while still extending battery life. Shown in FIG. 7 is an LED 202 mounted on a first side of a printed circuit board 200 which can have connectors 208 for wiring to the main circuit panel 270 shown in FIG. 12. The LED 202 can have a fixed spot size 205 as it enters the cranium of the patient. Alternatively, the spot size can be reduced or increased by selected amounts to either reduce the volume of brain tissue illuminated, or increase the volume. The LED panel shown in FIG. 7 can include sensor components such as EEG electrodes and/or photodetectors configured to detect light from the illuminated tissue within the cranium. The circuit panel 270 can include a wireless transceiver to transmit and receive data from the external controller within the tablet as described herein. The circuit panel 270 can also include a wired connector to connect the system to an external power source and the tablet used to control the system. As shown in FIG. 12, two manual switches 272, 274 can be used to actuate different power levels of the system. In this specific example a first switch selects between two different levels and the second switch selects among four different settings or sublevels for a total of eight different options. These switches can also be controlled remotely from a tablet as described herein. An LED 276 is used to indicate to a user that the power is on. A further switch 282 can turn on the wireless transmitter 280, which in this implementation, is a Bluetooth transceiver. LEDs 284 can further indicate the status of the transmitter. A central microprocessor 278 is programmed to control operations of circuit board 270. The power supply board 250 and controller board 260 shown in FIGS. 10 and 11 regulate power from the one or more batteries to the controller board. The on/off power switch 262 for the head worn device can be located on board 260 which includes an inductor 264 so that the voltage delivered to the LEDs does not vary as power is drawn from the batteries. These components are mounted on the head wearable device 110 with earphones 52 which are driven by the main controller board so that the patient can hear audio files used during therapeutic sessions. Alternatively, the electronics shown and described herein can be implemented using integrated circuit components to reduce size, weight and power requirements. An electronic sensor can monitor the voltage applied to one or more LEDs so as to record the amount of optical power delivered to the patient. The electronics can be implemented as an application specific integrated circuit (ASIC) or a system on chip (SOC) design.

Autism Spectrum Disorder (ASD) is a neurodevelopmental disorder characterized by diminished social functioning, inattentiveness, and linguistic impairment. While autism is likely to be a multi-causal disorder, research indicates that individuals with ASD frequently have mitochondrial disease which results in abnormalities of energy generation from food proteins. However, mitochondria in the brain might be able to produce energy molecules from a different source, such as light.

Using the wearable device 50, certain methods of the present disclosure may perform photobiomodulation (stimulating brain with light) and linguistic training simultaneously to treat children with ASD. The wearable device 50 may include several near infrared and/or red lights to stimulate the language area of the brain. These methods associated with the wearable device 50 may include determining an area of the head to position the wearable device 50 (e.g., the temporal lobe, the prefrontal cortex, and/or the occipital lobe) to output the infrared and/or red lights. The light absorbed by the brain tissue may increase the production of ATP, which may provide the neurons more energy to communicate with each other and provide increased brain connectedness. The wearable device 50 may simultaneously receive linguistic inputs from an application of a user device that is transmitted to the user via the headphones of the wearable device 50. The linguistic inputs may help facilitate language learning. Therefore, by providing these combined mechanisms (photobiomodulation and linguistic input), for example, to children diagnosed with ASD, may significantly improve lifelong outcomes. Further, the wearable device 50 may output meditations that may help reduce anxiety of the patient user (such as an ASD child), which may allow the user to better learn language and integrate socially.

Autism spectrum disorders are associated with brain inflammation, in particular, inflammation characterized by activation of brain macrophages (microglial activation) (see, e.g., Rodriguez et al., Neuron Glia Biol 2011, 7(204):205-213; Suzuki et al., JAMA Psychiatry 2013, 70(1):49-58; and Takano, Dev Neurosci 2015, 37:195-202, the entire contents of each of which are hereby incorporated herein by reference). Brain inflammation can be identified by the presence of delta waves (high voltage slow waves) during wakefulness that can be detected using electroencephalography (EEG). In healthy individuals, delta waves in EEG are detected during the period of restorative sleep, but not during wakefulness. Wakeful delta waves in EEG are associated with pathological conditions that are, in turn, associated with brain injury and inflammation characterized by activation of brain macrophages (microglial activation). Such pathological conditions include, among others, classical mitochondrial diseases like Alpers syndrome, traumatic brain injury and autism spectrum disorders (ASD). The presence of wakeful delta waves in an individual with a pathological condition described above indicates that healing activities normally confined to sleep were not sufficient for inhibiting brain inflammation.

Evidence also indicates that wakeful delta wave power is a reliable marker of brain inflammation and microglial activation. For example, symptomatic improvement in traumatic brain injury and genetic forms of mitochondrial brain disease is accompanied by a decrease in wakeful delta wave power. The presence of wakeful delta waves in an individual with ASD is indicative of brain inflammation.

Activation of microglia during brain inflammation results in synthesis and release of nitric oxide (NO) in the inflamed brain tissues. NO inhibits oxidative phosphorylation in the mitochondria by binding to the iron and copper atoms present in the mitochondrial electron transport chain complex IV cytochrome oxidase and inhibiting its activity (see, e.g., Mason et al., PNAS 2006 103(3):708-713, the entire contents of which are hereby incorporated herein by reference). Decreased levels of oxidative phosphorylation result in the increased levels of dissolved oxygen in the cell which, in turn, results in the increased levels of reactive oxygen species (ROS) and mitochondrial damage and fragmentation.

Near infrared light penetrates biological tissues, including bone structures such as the cranium. It can act to displace NO bound to the complex IV cytochrome oxidase, thereby reversing the effect of elevated NO levels and reversing the inhibition of oxidative phosphorylation. Restoration of mitochondrial oxygen consumption has the effect of stimulating healing of the inflamed tissues. Thus, without being bound by a specific pathway when other physiologic pathways, medications or therapeutic agents may alter the circumstances impacting treatment of a particular patient, it is believed that illuminating brain tissue of a subject with near infrared light can reverse inhibition of oxidative phosphorylation mediated by NO, stimulate oxidative phosphorylation and facilitate healing of inflamed brain tissues, thereby reducing brain inflammation. It is also believed, without being bound by a specific pathway as noted above, that the reduced inflammation of brain tissue resulting from illumination as described in the present application with red, near infrared light and/or infrared portions of the electromagnetic spectrum can cause a decrease in the wakeful delta waves, for example. Indeed, as indicated by the results of the clinical trial described herein, photobiomodulation therapy resulted in a statistically significant decrease in the delta waves in the treatment group as compared to a control group, which, in turn, was associated with a statistically significant reduction in autism symptoms.

Methods for providing cross-modal brain stimulation may include determining the light frequency, location of the LED lights (e.g., areas of the brain needing increased ATP, areas of the brain most likely to respond to light treatments, and/or areas of the brain associated with language (e.g., auditory cortex, Broca area, Wernike area)), whether ATP production increased, and the overall effect of the treatments. Accordingly, based on the determined overall effect on the brain, the wearable device may be dynamically adjusted on a user-specific basis.

The wearable device 50 may be specifically tailored for children with ASD, such that it improves language skills, alleviates anxiety, and/or reduces tantrums. Further, the wearable device 50 may be used on a daily basis, in the convenience of the family's home, without a need for a specially trained therapist. Moreover, the wearable device 50 may be non-invasive, may not require a prescription, and/or may lack side effects.

Methods for using the devices of the present disclosure may further include determining the location(s) of the light emitting diodes that may be used to stimulate specific brain areas responsible for language, comprehension, energy production, and/or for self-regulation (e.g., reducing anxiety). The methods may also include determining total power, power density, pulsing, and/or frequency. The total power may be 400-600 mW (0.4-0.6 W) with 100-150 mW per each of four panels. The power for each panel may be selectively stepped down to the 50-100 mW range, or increased to the 150-200 mW range depending on the age or condition of the patient. Each of these ranges may be further incremented in 10 mW steps during a treatment session or between sessions. The spot size of the light generated by each LED or laser can optionally be controlled by adjusting the spacing between the light emission aperture of the LED or by using a movable lens for one or more LEDs on each circuit board that can be moved between adjustable positions by a MEMS actuator, for example.

Further, the wearable device 50 may be comprised of a comfortable material for prospective patients. For example, the wearable device may be comprised of plastic, fabric (e.g., cotton, polyether, rayon, etc.), and/or the like. Because ASD patients in particular are especially sensitive, the aforementioned materials may be integral in allowing ASD patients to wear it for a sufficient amount of time without being irritable. Of course, the wearable device 50 may need to be both safe and comfortable. The electric components (e.g., processors, microphones, headphones, etc.) may be sewn into the wearable device 50 and may be difficult to reach by children, for example. A cloth or fabric covering can contain the head worn frame and optoelectronic components to the extent possible without interfering with the optical coupling of the LED to the cranium. Further, the weight of the wearable device 100 may be light enough to allow it to be worn comfortably. Moreover, the wearable device 100 may require a power source (e.g., one or more replaceable batteries) that allows it to be portable.

Regarding the linguistic inputs, a patient user device (e.g., a smartphone or tablet) may include an application that 1) performs language acquisition: (e.g., develops and records a vast number of short vignettes specifically designed to make syntactic structure transparent and teaches how to parse sentences); 2) involves a system of specifically designed mediations to alleviate anxiety; and 3) involves a system of musical rewards to keep users (children) interested and engaged.

The application may disambiguate syntactic structure of a language. Present research suggests that word learning spurt occurs after the children learn basic syntax (and it occurs at the syntactic-lexicon interface). Furthermore, without syntax children may not move beyond speaking 10-15 words, which may be used for simple labeling, but not to express their needs, wants and feelings. This means that there may be no ability for proper communication without learning syntax first. In addition, syntax may be necessary to parse the acoustic wave or sound that children hear into sentences and words. Syntax may also be necessary for specific word-learning strategies (e.g., syntactic bootstrapping).

Syntactic bootstrapping is a mechanism which children use to infer meanings of verbs from the syntactic clues. For example, when a child hears “Michael eats soup” this child infers that “eats” is a transitive verb. A classic example used by a famous psycholinguist professor Leila Gleitman is the made-up verb “Derk”. By putting this verb in several syntactic contexts, the meaning of the verb becomes transparent “Derk! Derk up! Derk here! Derk at me! Derk what you did!” Dr. Gleitman argued that children infer the meanings of verbs from hearing them in different syntactic contexts. In addition, Dr. Pinker argued that children also use semantic bootstrapping (contextual clues) to infer meanings of the words. Therefore, there are several mechanisms (most likely innate) available to a typical child while learning language. Overall, there is scientific consensus that typical children learn language by specifically focusing on syntactic and semantic clues of speech.

However, studies suggest that children who are on the autism spectrum cannot always extract syntactic structure and semantic contexts from the imperfect linguistic input they receive. Usual linguistic input is too messy, incomplete and confusing for them. People frequently speak in fragments of sentences, switch between topics, use incorrect words or use words in incorrect forms. Human speech may be too messy to allow for simple learning based on this type of speech alone. Neurotypical children can still extract syntactic structure from this messy input by being predisposed to pay attention to specific syntactic cues (e.g., to look for nouns and verbs in the string of speech). When children grasp syntactic structure of a language, they learn to parse sentences, and therefore, acquire more words. Several studies corroborated this hypothesis that massive word learning happens at this syntax-lexicon interface, including studies with children on the spectrum.

Many children suffering from ASD seem to be unable to move beyond simple labeling, are unable to speak in full sentences, and therefore are unable to communicate effectively. There are many reasons for this difficulty, one of them is that those children do not usually pay enough attention to speech and communication, and therefore they do not pay enough attention to syntactic clues and are not able to parse individual sentences. However, without grasping syntactic structure of the language, word learning beyond simple labeling becomes impossible, specifically, acquisition of verbs may become impossible. Timely acquisition of verbs (not just nouns to label objects around them) may be critical for ASD children, as research shows that the best predictor of future integration with the neuro-typical community (and normal functioning) is speaking full sentences by 5 years of age. Therefore, specifically, the problem is that children with ASD are not focused on the language enough to extract syntactic features of words, to parse sentences and to attend to syntactic and semantic clues of speech. Therefore, their word learning is delayed.

Accordingly, the aforementioned application may calibrate the imperfect linguistic input for ASD children, thus, making syntactic structure as transparent as possible. For example, the child will hear a noun: “dog”, then she will hear “1 dog, 2 dogs, 3 dogs, 4 dogs, 5 dogs”. then she will hear “my dog is brown”, “my brown dog is cute”, “my brown dog is small”, “I have a small, cute, brown dog”, “my dog barks,” “dogs bark” “dogs chase cats”, “dogs eat meat”, “I have a dog,” and so on. By putting the same word in different syntactic contexts over and over again we will flood the child with the information about linguistic markers (syntactic roles in the sentences, countable, noun, animate/inanimate and so on).

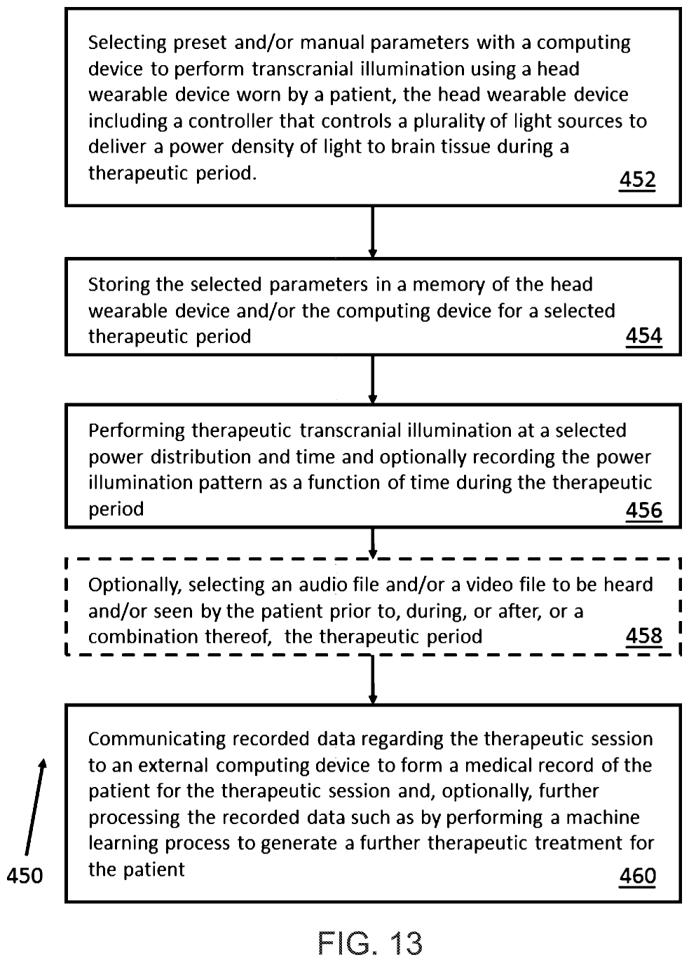

Therefore, the application may “wake up” (activate) language learning and make a child pay attention to the syntactic cues of the linguistic input. Further, the application may be refined by observing the behavior of the users and recording their improvements. A method for treatment 450 is described in connection with the process flow diagram of FIG. 13 wherein preset or manually entered parameters 452 can be entered by touch actuation on the tablet touchscreen so that the system controller can actuate the illumination sequence. These parameters are stored 454 in memory. The software for the system then executes stored instructions based on the selected parameters to provide transcranial illumination 456 for the therapeutic period. The system can utilize optional audio or video files 458 in conjunction with the therapy session. The system than communicates the recorded data 460 for the therapeutic session for storage in the electronic medical record of the patient. The data can be used for further analysis such as by application of a machine learning program to provide training data.

The following describes an example of a battery powered system as previously described herein where one or two 9 volt batteries are inserted into battery holders in the side and rear views showing the LED case design shown in the figures.

If the LED is uncased, a small tube can be used to ensure that it remains centered and held securely in place. This tube can fit through a hole in the foam band for proper location and is ⅜″ outside diameter. The PCB serves as a backing on the foam and allows clearance for the connecting cable. The same type of construction can be applied to the electronics mounted area, the battery, and the speakers. Sensors used to measure characteristics of the patient during use, such as EEG electrodes, photodetectors and/or temperature sensors can be mounted to the circuit boards carrying the LEDs or laser diodes as described generally herein. Detected electrical signals from the sensors can be routed to the controller board and stored in local memory and can also be transmitted via wireless transmission to the external tablet device so that a user or clinician can monitor the therapeutic session and control changes to the operating parameters of the system during use.

The electronics can comprise three or more separate PCB configurations with the LED PCB having (6) variations for the associated positions on the head. There can be two LED PCB boards on each side (front and rear) with at least one illuminating the temporal lobe on each side and at least one board centered for illuminating the frontal lobe. One or two boards can conform to one or both of the parietal lobe and the occipital lobe.

The system is fitted on the head of a patient and radiates energy via IR LEDs at 40 Hz into the patient's head, for example. The IR LEDs are split into six boards with each containing one IR LED. The LED utilized for preferred embodiments can be the SST-05-IR-B40-K850.

The LED boards can illuminate during the on-time of the 40 Hz signal. The duty cycle of the 40 Hz signal will be equal to the power setting. For example, a power setting of 25% will require a 25% duty cycle for the 40 Hz.

One or more 9V batteries can be the system's source of power. A buck converter reduces the 9V from the battery to 2.5V for the LEDs. One or more batteries of different voltages can be employed particularly where different batteries can be used for the light emitters and powering the circuitry.

In this section, note the calculation of the LED's absolute maximum optical flux output assuming that they are the only components powered by a single 9V battery.

Table 1 shows the current limits of important components. These current limits cannot be violated without the risk of permanent damage to the component.

| Device Specification | Current Limit (A) |
|---|---|
| Absolute Maximum Battery | 1.0 |
| Discharge | |
| Absolute Maximum Buck | 2.5 |
| Converter Current Output | |
| Absolute Maximum IR LED | 1.0 |
| Current | |

Conservation of energy dictates that the current sourced by the buck converter will not be the same as the current sourced by the battery. Equation 1 calculates the current drawn from the battery (I_{BATT}).

$${I}_{BATT} = \frac{{V}_{LED \_ PWR} \times {I}_{LED \_ PWR}}{η \times {V}_{BATT}}$$

where V_{LED_PWR} is the LED supply voltage (2.5 V), I_{LED_PWR} is the buck converter output current, η is the efficiency (minimum of 0.85), and V_{BATT} is the battery voltage.

The efficiency of the buck converter changes over the output current range. The minimum efficiency is 0.85 at the maximum current of 2.5 A.

Note that the battery voltage is inversely proportional to the battery draw. For a fixed load, the battery will draw more current as the battery discharges. Therefore, a minimum battery voltage must be specified and observed by the system microcontroller to avoid exceeding the battery's maximum discharge current. Table 2 demonstrates how battery current draw increases as the battery discharges. Each battery draw value is calculated with Equation 1 with the following values: η=0.85, V_{LED_PWR}=2.5V, I_{LED_PWR}=2.5 A, and the battery voltage for V_{BATT}.

| | Battery |
|---|---|
| | Draw |
| Scenario | (mA) |
| Fully Charged Battery at 9 V | 816 |
| Intermediate Charge at 7.5 V | 980 |
| Absolute Minimum | 1000 |
| Battery Voltage, 7.35 V | |

Use Equation 2 to calculate the absolute minimum battery voltage, V_{B_AM}. Use the same values as before, but let I_{B_MAX}=1. Battery current draw reaches 1.0 A when the battery voltage discharges to 7.35V, therefore the LEDs must be turned off to avoid exceeding the L522 battery maximum discharge specification of 1.0 A. The buck converter supplying 2.5 A at 2.5V with a battery voltage below 7.35V risks permanent damage to the battery.

$${V}_{B \_ AM} = \frac{{V}_{LED \_ PWR} \times {I}_{LED \_ PWR}}{η \times {I}_{B \_ MAX}}$$

Equation 2. Absolute Minimum Batters Current Draw

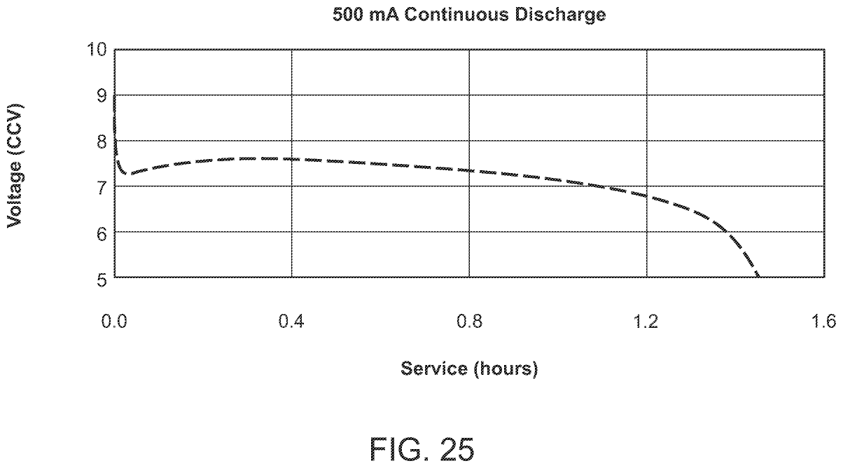

The absolute minimum battery voltage also affects battery life. FIG. 25 illustrates a discharge curve for the (Energizer L522) battery and it demonstrates that a lower absolute minimum battery voltage prolongs battery life. With a 7.35V absolute minimum battery voltage, the LEDs can be safely powered for approximately 24 minutes (if the battery was drawing 500 mA instead of 1.0 A). Thus, a lower absolute minimum battery voltage is beneficial.

The 2.5 A sourced by the buck converter must be shared amongst six boards (LED). Thus 2.5 A/6=416 mA from the buck converter per LED.

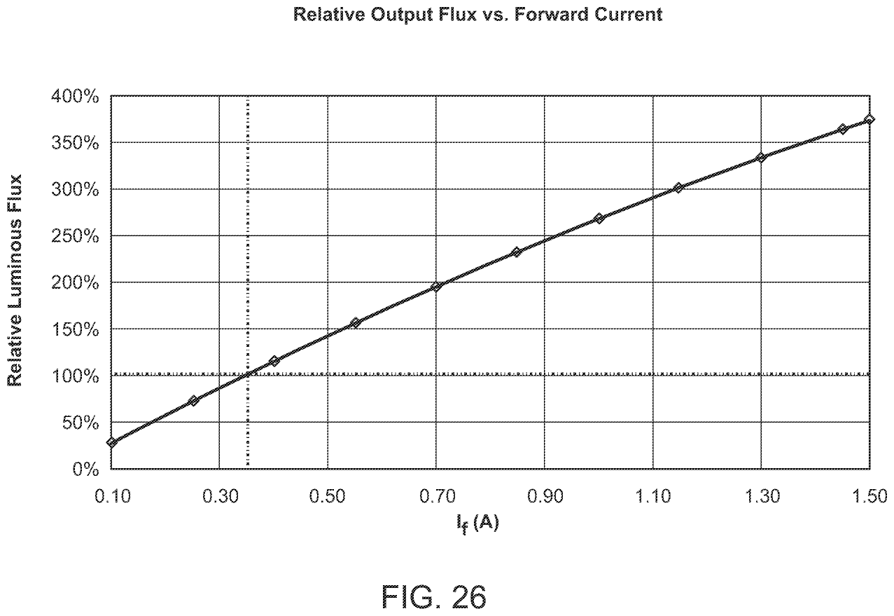

FIG. 26 illustrates the manufacturer's graph of optical flux normalized at 350 mA. The manufacturer datasheet states that the optical flux at 350 mA ranges between 265 mW and 295 mW. At 416 mA, the optical flux is approximately 110% the optical flux at 350 mA. Using the worst-case flux output of 265 mA, the optical flux at 416 mA is 265 mW×1.1=291.5 mW or approximately 292 mW.

The duty cycle of the 40 Hz will attenuate the optical flux. Equation 3 shows how to calculate the average flux for a single pulsed LED. E_{e_pulse} is the optical flux during the pulse and D_{40HZ} is the 40 Hz duty cycle. The range of D_{40HZ} is a number between 0 and 1 inclusive.

E_{e_average}=D_{40HZ}×E_{e_pulse}

Equation 3. Average irradiance for a single LED.

As an example, Table 3 lists the optical flux for each power setting.

TABLE 3
Power Settings
at Maximum
System Power.

| | Optical |
|---|---|
| | Flux |
| Power | Output |
| Setting | (mW) |
| 2% | 5.84 |
| 4% | 11.68 |
| 6% | 17.52 |
| 8% | 23.36 |
| 16% | 46.72 |
| 25% | 73 |
| 50% | 146 |
| 100% | 292 |

The output optical flux decreases with temperature and must be de-rated accordingly. Sources of heat to take into account are the LEDs' self-heating and the heat from the patient's head. For the purposes of this analysis, assume the patient's head is at body temperature, 37° C.

TABLE 4
Temperature Related Coefficients

| Parameter | Value |
|---|---|
| Temperature Coefficient of | −0.3%/° C. |
| Radiometric Power | |
| Electrical Thermal Resistance | 9.2° C./W |

Table 4 above lists two thermal coefficients. The thermal resistance of the LED can be understood as for every watt consumed by the LED, its temperature will rise by 9.2° C. The third graph below shows normalized V-I characteristics of the LED relative to 350 mA at 2V (at 350 mA, forward voltage ranges between 1.2V and 2.0V, but here we continue to use worst-case value of 2.0 V).

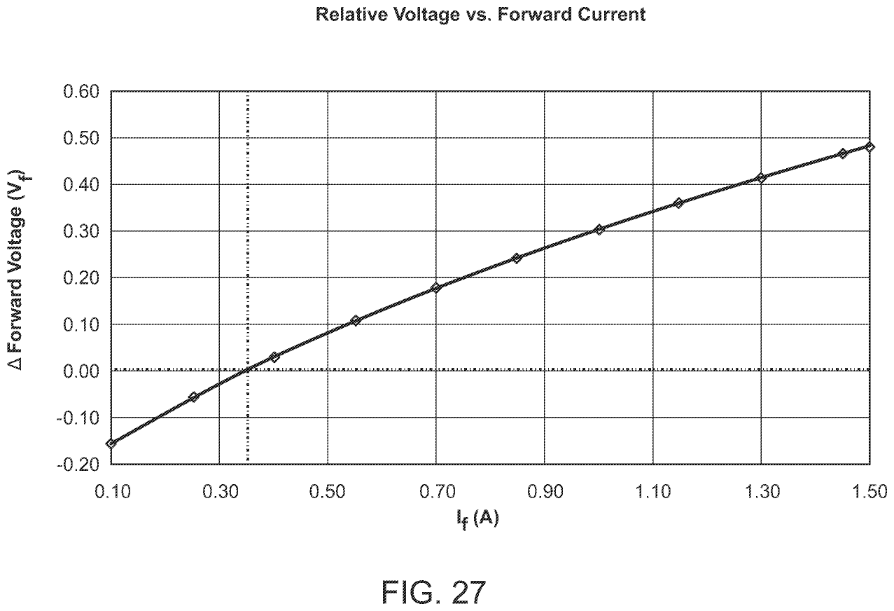

At 416 mA (the maximum current available per LED), FIG. 27 illustrates that the forward voltage is approximately 2V+0.04V=2.04V. Using Equation 4, the temperature rise due to self-heating is 7.8° C. at a 100% 40 Hz duty cycle.

T_{Δ}_{_{LED}}=V_{f}×I_{f}×D_{40HZ}×9.2

Equation 4. Temperature Rise due to Self-Heating

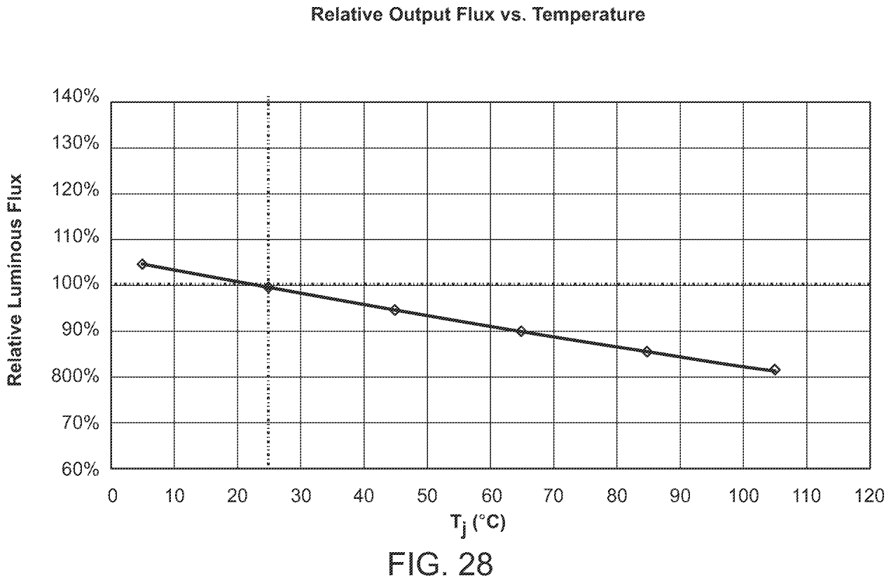

The LED can rise to a temperature of T_{LED}=37° C.+7.8° C.=44.8° C. The optical flux vs temperature graph in FIG. 28 is normalized to 25° C. Using the temperature coefficient of radiant power from Table 4 and Equation 5, the change in radiant power due to temperature is −5.94%. Therefore, de-rating the worst-case optical flux of 292 mW derived above by 5.94% yields approximately 275 mW.

PO_{Δ%}=(T_{LED}−25° C.)×(−0.3%/° C.)

Equation 5. Change in Output Flux due to Temperature.

Note that the system also provides a temperature coefficient for forward voltage. Forward voltage decreases with temperature rise. For a worst-case analysis, the decrease in forward voltage due to temperature can be ignored.

An optical flux of 275 mW is the minimum absolute maximum that can be achieved if the buck converter and the battery are pushed to their limits assuming that the battery is only supplying power to the LEDs.

Since the battery may also be powering the digital logic which includes the microcontroller, the Bluetooth module or other wireless connection, etc. the LEDs cannot draw the 1.0 A maximum from the battery.

The steps below are an effort to summarize the approach described above.
1. Start by selecting the target current for a single LED, I_{f}.
2. The current sourced by the buck converter will be I_{LED_PWR}=6×I_{f}. If I_{LED_PWR} exceeds 2.5 A, you must decrease I_{f}.
3. Use Equation 2 to calculate the minimum safe battery voltage to ensure desired battery life and safe operating conditions. For efficiency, either use the worst-case value of 0.85 or select the closest efficiency for your value of I_{LED_PWR} from Table 5.

TABLE 5
Buck Converter Efficiency for
Different Output Currents

| Buck | |
|---|---|
| Converter | |
| Output, | Efficiency, |
| I_{LED}_PWR (A) | η |
| 2.5 | 0.85 |
| 2.0 | 0.871 |
| 1.5 | 0.89 |
| 1.0 | 0.91 |
| 0.5 | 0.926 |
| 0.25 | 0.91 |
| 0.125 | 0.88 |

4. Use graph to approximate the optical flux output at I_{f}.
   a. Note: Graph is normalized to optical flux of 265 mW at 350 mA.
5. Use graph to approximate the forward voltage at I_{f}.
   a. Note: Graph is normalized to 2.0V forward voltage at 350 mA.
6. Calculate the self-heating temperature rise, T_{Δ_LED}, using Equation 4. Use D_{40HZ}=1 for 100% 40 Hz duty cycle as the worst-case temperature rise.
7. De-rate the optical flux for a T_{Δ_LED} rise over ambient temperature. Use 37° C. for ambient temperature. This de-rated optical flux is the maximum flux output for a single LED.

Table 6 gives examples of target LED current and the resulting system specification. Allow a 100 mA margin on the battery draw for supply logic. Values calculated in Table 6 assume worst-case efficiency of 0.85.

TABLE 6
LED Current Target Examples.

| | | | | | Flux |
|---|---|---|---|---|---|
| | Absolute | | | Flux | Output |
| Target | Minimum | Battery | LED | Output | per LED |
| LED | Battery | Draw | Temperature | per | (temperature |
| Current | Voltage | at 6.5 V | Rise | LED | adjusted) |
| (mA) | (V) | (mA) | (° C.) | (mW) | (mW) |
| 100 | 1.765 | 271 | 1.7 | 57 | 55 |
| 200 | 3.529 | 543 | 3.5 | 133 | 127 |
| 300 | 5.294 | 814 | 5.4 | 207 | 196 |
| 339 | 5.982 | 900 | 6.2 | 236 | 223 |

The maximum target LED current is 339 mA resulting in a temperature adjusted flux output of 223 mW. Table 7 demonstrates how the 40 Hz duty cycle attenuates the LED output flux.

TABLE 7
Power Settings for Realistically
Maximizing Flux Output

| | Optical |
|---|---|
| | Flux |
| Power | Output |
| Setting | (mW) |
| 2% | 4.46 |
| 4% | 8.92 |
| 6% | 13.4 |
| 8% | 17.8 |
| 16% | 35.7 |
| 25% | 55.8 |
| 50% | 111.5 |
| 100% | 223 |

EEG can be used to augment the use of TPBM to reduce symptoms of autism, for example, and this procedure is described in further detail below.

The head wearable device reduces symptoms of autism by applying tPBM to stabilize functional brain connectivity, while using EEG data as a measure of the efficacy of tPBM and as a guide for continuous applications. The head wearable device can include EEG electrodes situated on one or more of the light emitter printed circuit boards as described herein. Between one and six EEG electrodes can be mounted on one or more of the light emitter panels so that they are interleaved between the light emitters or surround them so as to detect brain wave signals occurring during illumination.

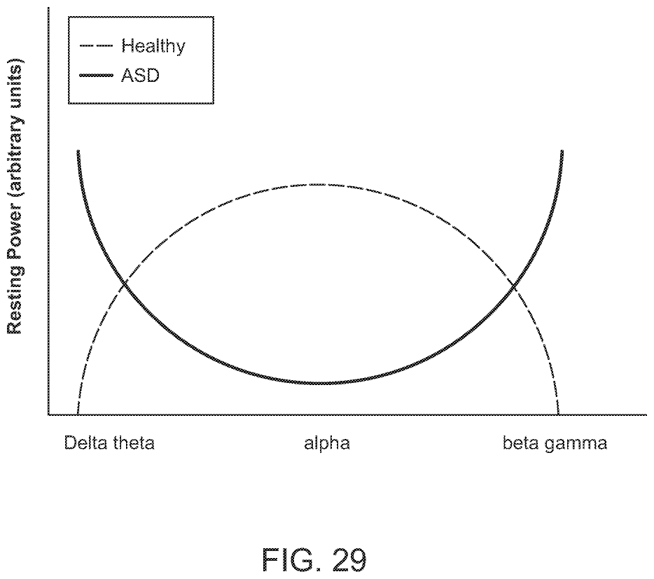

Autism (ASD) is a life-long disorder characterized by repetitive behaviors and deficiencies in verbal and non-verbal communication. Resent research identified early bio-markers of autism, including abnormalities in EEG of ASD infants, toddlers and children as compared to typical children. For example, children diagnosed with ASD present with significantly more epileptiforms (even, when they do not develop seizures), some researchers report as many as 30% of ASD children present with epileptiforms (e.g., Spence and Schneider, Pediatric Research 65, 599-606 (2009). A recent longitudinal study (from 3 to 36 months) detected abnormal developmental trajectory in delta and gamma frequencies, which allow distinguishing children with ASD diagnosis from others (Gabard-Durnam et al 2019). Short-range hyper-connectivity is also reported in ASD children. For example, Orekhova et al (2014). showed that alpha range hyper-connectivity in the frontal area at 14 months (and that it correlates with repetitive behaviors at 3 years old). Wang et al (2013), has indicated that individuals with ASD present with abnormal distribution of various brain waves. Specifically, the researchers argued that individuals with ASD show an excess power displayed in low-frequency (delta, theta) and high-frequency (beta, gamma) bands as shown in FIG. 29, and reduced relative and absolute power in middle-range (alpha) frequencies across many brain regions including the frontal, occipital, parietal, and temporal cortex. This pattern indicates a U-shaped profile of electrophysiological power alterations in ASD in which the extremities of the power spectrum are abnormally increased, while power in the middle frequencies is reduced.

Duffy & Als (2019) argued, based on EEG data, that ASD is not a spectrum but rather a “cluster” disorder (as they identified two separate clusters of ASD population) and Bosl et al Scientific Reports 8, 6828 (2018) used non-linear analyses of infant EEG data to predict autism for babies as young as 3 months. Further details concerning the application of computational methods of Bosl can be found in US Patent publication 2013/0178731 filed on Mar. 25, 2013 with application Ser. No. 13/816,645, from PCT/US2011/047561 filed on Aug. 12, 2011, the entire contents of which is incorporated herein by reference. This application describes the application of machine learning and computational techniques including the use of training data stored over time for numerous patients and conditions that can be used to train the a machine learning system for use with the methods and devices described herein. A neural network can be used for example to tune the parameters employed for transcranial illumination of a child at a certain age range undergoing treatment for autism. An array of 32 or 64 EEG channels can be used with electrodes distributed around the cranium of the child. Overall, the consensus is that ASD is a functional disconnectivity disorder, which has electrophysiological markers, which can be detected through an EEG system. Dickinson et al (2017) showed that at a group level, peak alpha frequency was decreased in ASD compared to TD children.

Transcranial photobiomodulation as described herein is used to treat many neurological conditions (TBI, Alzheimer, Depression, Anxiety), and is uniquely beneficial to autism, as it increases functional connectivity AND affects brain oscillations (Zombordi, et al, 2019; Wang et al 2018). Specifically, Zomonrodi et al Scientific Reports 9(1) 6309 (2019) showed that applying tPBM (LED-based device) to Default Mode Network increases a power of alpha, beta and gamma, while reduces the power of delta and theta (at resting state). Wang et al (2018) also showed significant increases in alpha and beta bands. Finally, Pruitt et all (2019) showed that tPBM increases cerebral metabolism of human brain (increasing ATP production).

Thus, preferred embodiments use a system that correlates continuously collected EEG data with observable symptoms (as reported by the parents) and use EEG to guide application of LED based tPBM. The symptoms provided by parents can provide ranked data can be used to formulate the parameters for a therapy session.

LED based tPBM can be applied to Default Mode Network (avoiding central midline areas) as well as Occipital lobe, and Broca area (left parietal lobe) as well as Wernike area (left temporal lobe).

Stimulating DMN (and simultaneous stimulation of frontal lobe with occipital lobe) increases long-range coherence. Stimulating language producing areas (e.g., Broca and Wernike areas with DMN) has been shown to facilitate language production in aphasic stroke patients (Naeser, 2014).

The device performs EEG measurements in combination with photobiomodulation therapy:

1. Analyze initial EEG data for epileptiforms, long-range coherence and hemispheric dominance.

2. Correlate EEG data with observed symptoms.

3. Based on the observed symptoms and the EEG data, the head wearable device can apply tPBM. For example, for children with severe repetitive behaviors and strong delta and theta power in the prefrontal cortex, the device stimulates prefrontal cortex to increase power within alpha and beta frequency band (and decrease power of delta and theta bands). For children who struggle with language, the device can stimulate DMN and Broca and Wernike areas. For children with various and severe symptoms, the device can stimulate all identified targeted areas (DMN, Broca, Wernike, occipital lobes).

4. The device can adjust power gradually and increasing it until the minimal change in brain oscillation is detected. This thresholding avoids applying too much power to a developing brain. The device operates at the lowest power that achieves the desired oscillation.

5. As the symptoms improve and the measured EEG signal stabilizes, the power level of the device can be gradually reduced. This system can be automated to control each therapy session.

6. Machine learning algorithms analyze EEG data and behavioral data, and the power alterations provided by the algorithm in the form of guidance to parents (and therapists), as well as indicate further improvements in the therapy being given to the patient.

7. As the symptoms sufficiently improve (expected improvement is within 8 weeks based on Leisman et al 2018), the device controls a break from tPBM and collect only EEG and behavioral symptoms to monitor for possible regression.

8. If any regress is detected, the device can instruct that tPBM is gradually resumed.

The device can apply tPBM to DMN, occipital lobe as well as to Broca and Wernike areas. The device collects EEG signals from prefrontal cortex, occipital cortex and temporal cortex (left and right to monitor hemispheric dominance observed in ASD children). The platform connected to the device can conduct initial assessment of behavioral symptoms (to be correlated with EEG data) as well as ongoing collection of symptoms (allowing for continuous correlations with EEG). Therefore the platform will continuously measure the efficacy of tPBM and personalization can be developed. Initially, a baseline is established by two separate measures. First, functional brain connectivity and brain oscillations baselines can be established in targeted brain areas (e.g., F1 & F2, T3 & T4, O1 & O2) prior to using treatment. Second, baseline demographic information (age, gender, race, etc), most concerning symptoms, and medical history (e.g., known genetic mutations, mitochondrial dysfunctions, gastroenterological symptoms, asthma, epilepsy, medications taken on regular basis) of each child is collected from parents. After the initial low-dosage treatment is administered several times (>3), a child's brain oscillations can be measured in order to establish the trend for reduction of delta brain waves, which is needed in order to detect the treatment's effect on the brain's electrophysiological activity and penetration of light through the skull. Separately, data can be collected from parents about the child's behavioral symptoms, including language, responsiveness, aggression, self-injurious behavior, irritability, and sleep disturbances. An AI algorithm, described in further detail below, processes collected data to determine the combination of effectiveness (as marked by behavioral symptoms and EEG data) as well as tolerability (as marked by behavior, reported by parents) to compute an optimal dosage (which include total power, time administered and frequency of pulsing). For example, the device used in some embodiments uses 40 HZ pulsing, which usually increases focus. However, for hyper-active children 10 HZ pulsing or continuous wave administration can be used and can be effective. To further improve personalization features, the system can be programmed to adjust for skin color based on the timing and strength of dosage. Darker skin pigments absorb light more than lighter skin, therefore fewer photons are likely to reach the brain. In the clinical study, children with darker skin showed improvement later than children with lighter skin, thereby indicating a need to adjust dosage based on skin absorption. Therefore, they might need longer usage of the device at a given dosage to detect improvements. The control software for the device can be programmed for such personal characteristics as race and ethnicity.

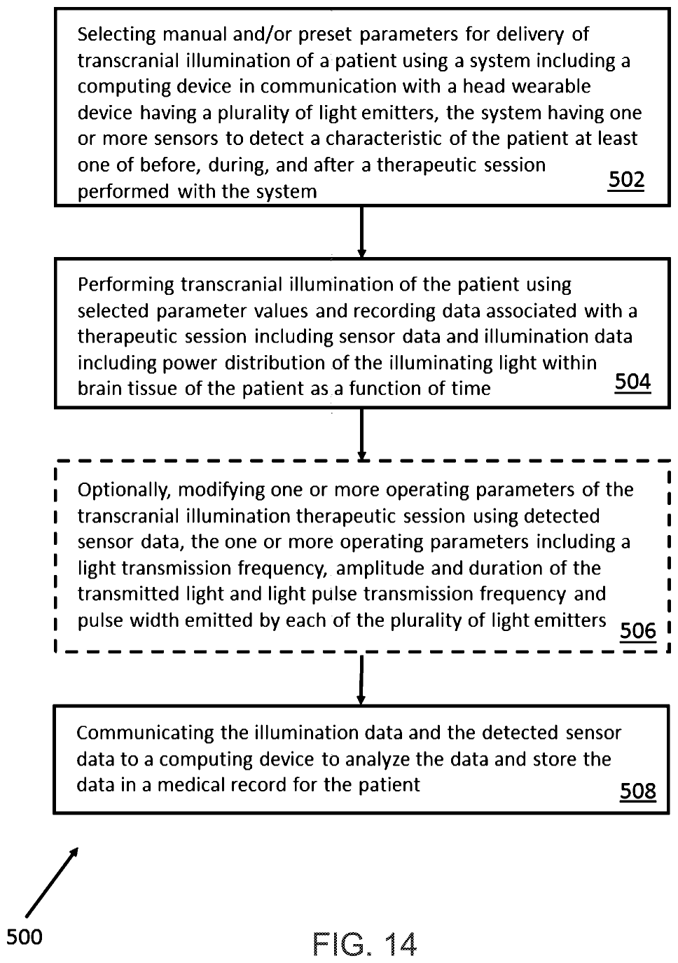

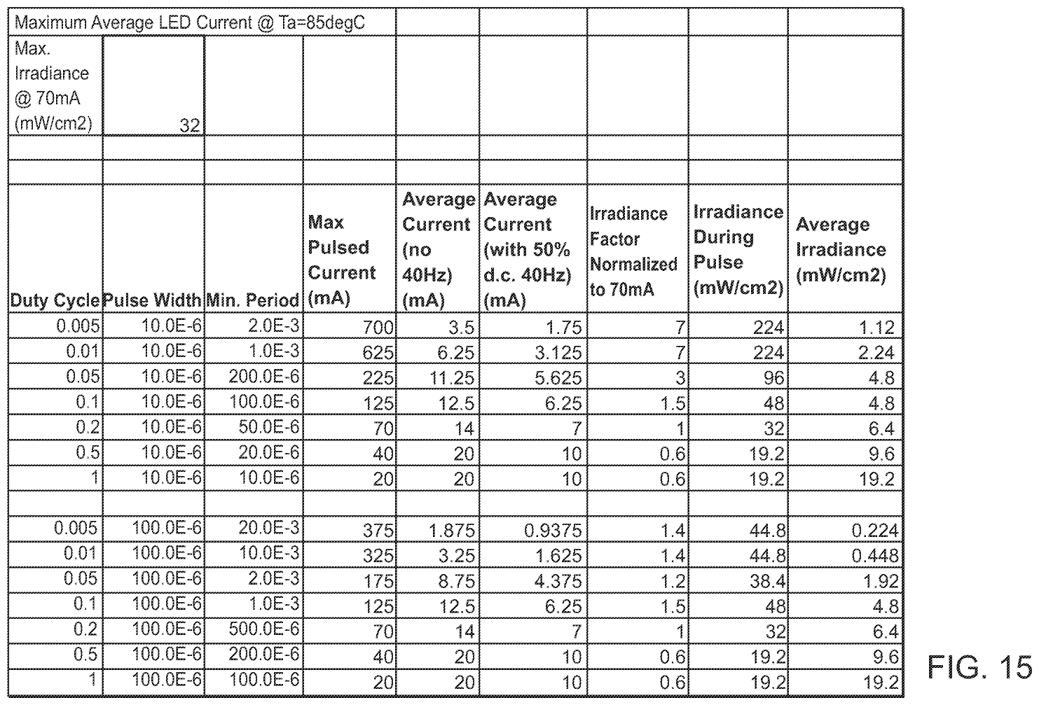

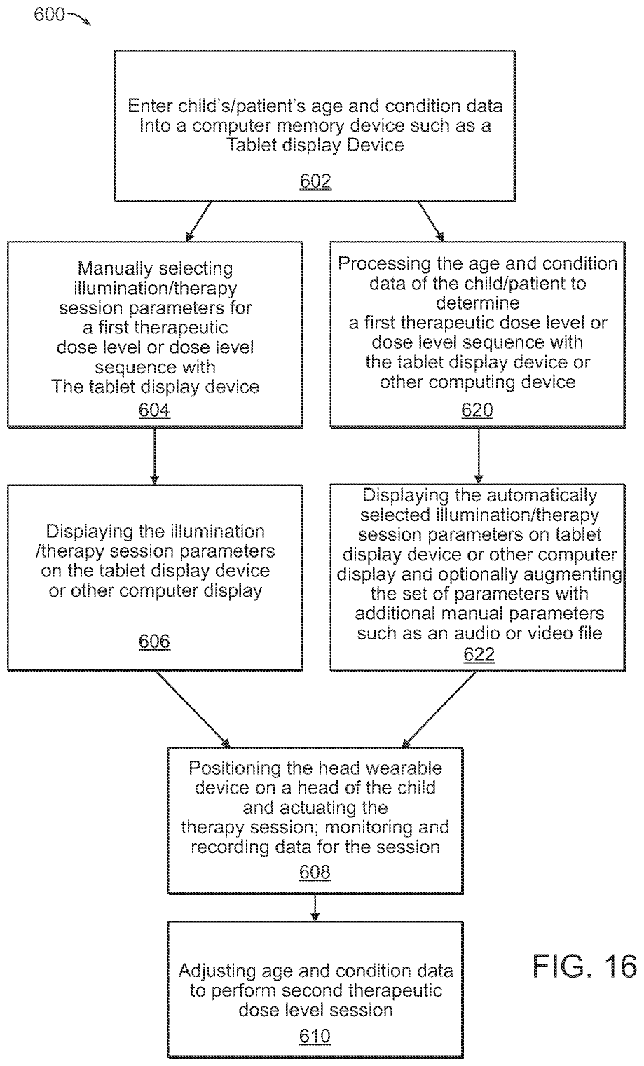

The process flow diagram in FIG. 14 illustrates the method 500 of performing transcranial illumination in combination with the use of one or more sensors to measure characteristics of the brain to monitor the treatment and detect changes in tissue that indicate a response during one or more sessions. Preferred embodiments can utilize an EEG sensor array with the head wearable device to measure brain electric field conditions where manual or preset parameters are selected 502 for a therapeutic session. The system performs transcranial illumination 504 and data is recorded such as EEG sensor data. Depending on the measured data and condition of the patient, the system can automatically adjust operating parameters or they can be manually adjusted 506 by the clinician. The data can be communicated 508 to the computing device such as the control tablet device and stored in the electronic medical record of the patient. This can be transmitted by communication networks to a hospital or clinic server for storage and further analysis as described herein. Shown in FIG. 15 is a table with exemplary values for illumination conditions that can be employed by the system. These parameters typically fall within a range of values that the system can use that extend between a minimum threshold and a maximum threshold. These thresholds can be age dependent as the thickness and density of the cranium of a child increase with age as described in Smith et al, “Automated Measurement of Cranial Bone Thickness and Density from Clinical Computed Tomography,” IEEE conference proceedings Eng Med Biol Soc. 2012: 4462-4465 (EMBC 2012), the entire contents of which is incorporated herein by reference. Thus, an age dependent quantitative rating can be associated with each patient that is used to define the illumination parameters used for that patient. Note that different lobes of a child may increase in thickness and/or density at different rates over time. Thus, the power density to be delivered to a child at age 4 will be less than that used for a 5 or 6 year old, for example.

Thus, an operating module of the software can be programmed to retrieve fields of data or data files from a patient data entry module that can include patient information and other initial observations of parents or clinicians regarding a child's age, condition, medical history including medications that may impact a further diagnostic or therapeutic program. FIG. 16 illustrates a process flow diagram for a method 600 of selecting and optimizing parameters over multiple therapeutic sessions including manual and automated selection tracks. Initially, patient data related to a child or adult patient (such as age or condition) can be entered by a user into a memory of a computing device (step 602). For example, data can be entered by a user through the GUI 160 of the remote computing device 150 (such as a tablet computing device) and stored in the memory 156 as described previously in relation to FIG. 4. The method 600 can then follow one of two tracks. In one embodiment, the user can manually select illumination or therapy session parameters for a first therapeutic dose level or dose level sequence based upon the patient data (step 604). For example, the user can manually select parameters from menu or other displays on the GUI 160 of the remote computing device 150. Then, the illumination and/or therapy session parameters (which may include user-selected parameters and other parameters whether automatically determined or set by default) can be displayed on the computer display (step 606). For example, the parameters can be displayed on the visual display device 152. The device can also be programmed to operate a linguistic and/or visual message therapy module that communicates auditory and/or visual messages to the patient during a therapy session.

In an alternative embodiment, the parameters can be set algorithmically or automatedly. The processor of the computing device can process the patient data (including, for example, age and condition data) to determine the first therapeutic dose level or dose level sequence (step 620). For example, the processor 155 of the remote computing device 150 can analyze and process the patient data. Then, the automatically selected illumination and therapy session parameters (as well as other session parameters) can be displayed on the display associated with the computing device (step 622). Optionally, the set of automatically selected parameters can be augmented in this step with additional manual parameters such as an audio or video file used as part of the therapeutic session.

Whether the parameters are determined automatically or manually, the head wearable device can then be positioned on the head of patient (e.g., a child or adult) and the therapy session can be actuated based on the session parameters (step 608). Data related to the patient or device during the session can be monitored and recorded. Then, the patient data (e.g., age or condition data) can be adjusted to optimize session parameters for future (i.e., second, third, or more) therapeutic sessions (step 610).

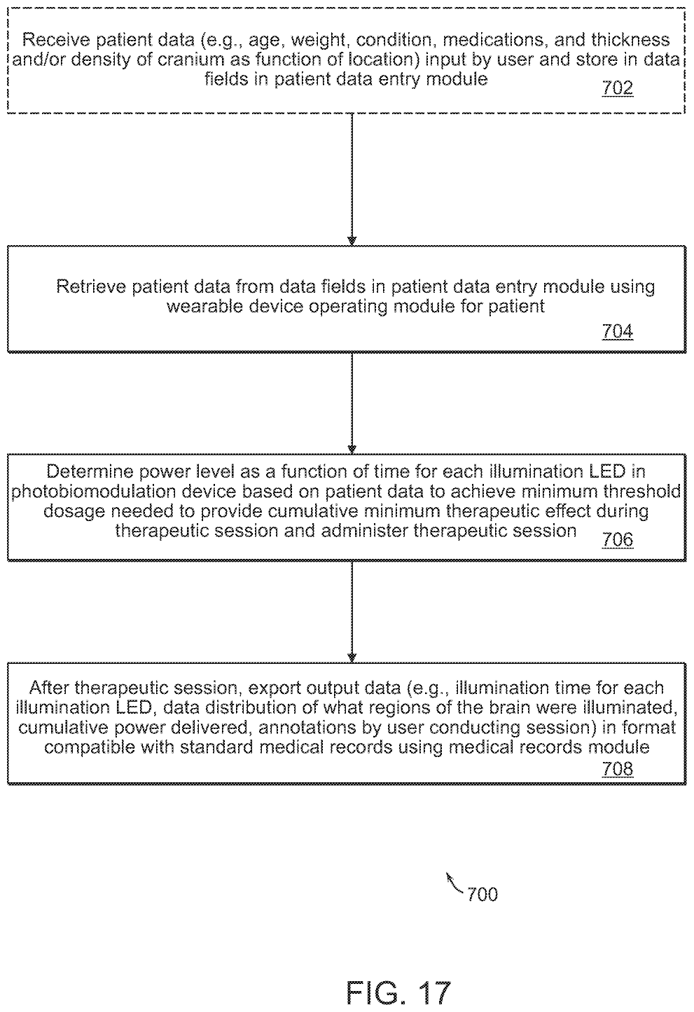

FIG. 17 illustrates a process flow diagram for a method 700 for administering a therapeutic session to a patient in accordance with various embodiments described herein. As an optional first step, patient data can be input by a user to a computing device and stored in data fields in a patient data entry module resident in the computing device or a server device (step 702). Relevant patient data entered in this step can include patient age, weight, physical or mental condition, medication history or regimen, and a data map of cranial thickness or density as a function of location on the patient's cranium. For example, the patient data entry module can reside in the memory 156 of the remote computing device 150, and patient data can be entered using the GUI 160 such as by using a keyboard, mouse, or multi-point touch interface 420. This step may be considered optional as the patient data for a particular patient may already be resident in patient data entry module (e.g., the data may have been entered during previous sessions and need not be re-entered). The patient data is then retrieved from the data fields in the patient data entry module using the wearable device operating module (step 704). The wearable device operating module can determine a power level as a function of time for each illumination LED 115a-115e in the array of the photobiomodulation device 110 based on the patient data to achieve the minimum therapeutic effect during the therapeutic session. Once the power levels are determined, the therapeutic session can be administered to the patient (step 706).

After concluding the therapeutic session, output data can be exported in a format compatible with standard medical records using a medical records module (step 708). Output data can include the illumination time and/or power for each individual illumination LED, a data distribution of which regions of the brain were illuminated, the cumulative power delivered, or annotations from a user conducting the session such as a medical professional. The data can be time-course data including time stamps that record when observations or other data events occurred within the therapeutic session.

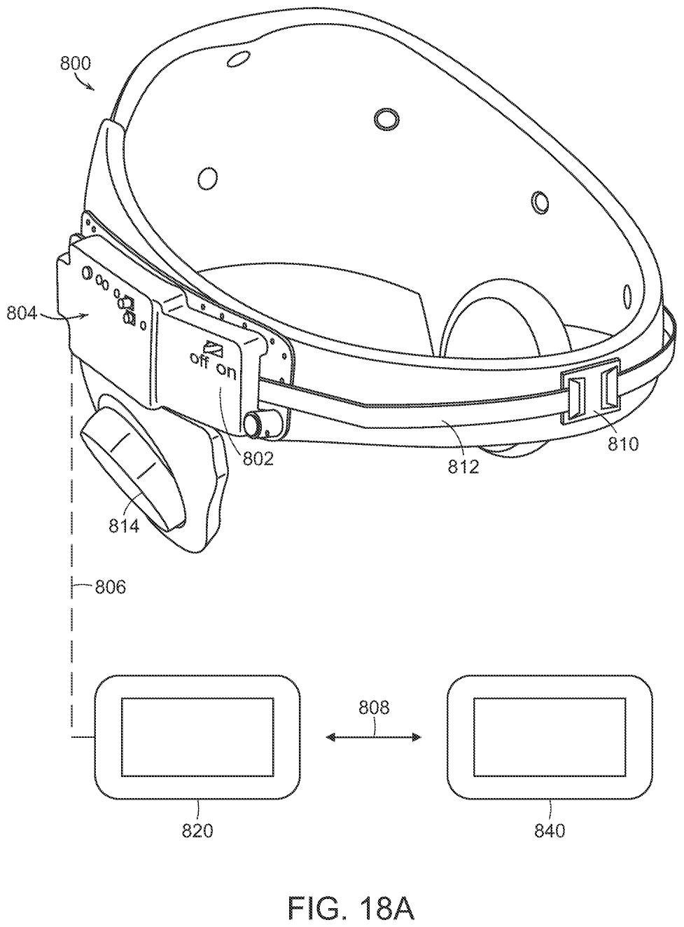

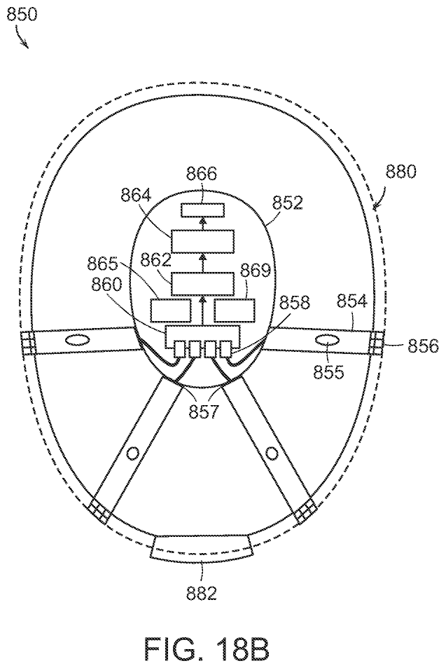

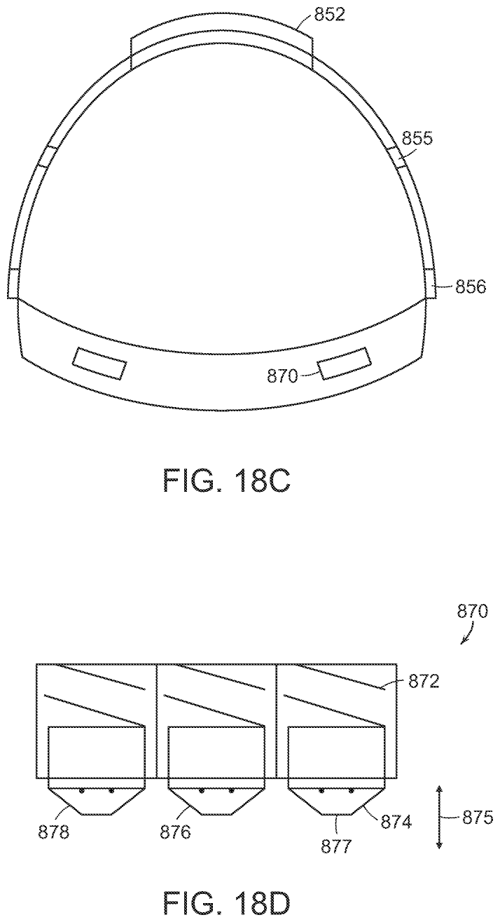

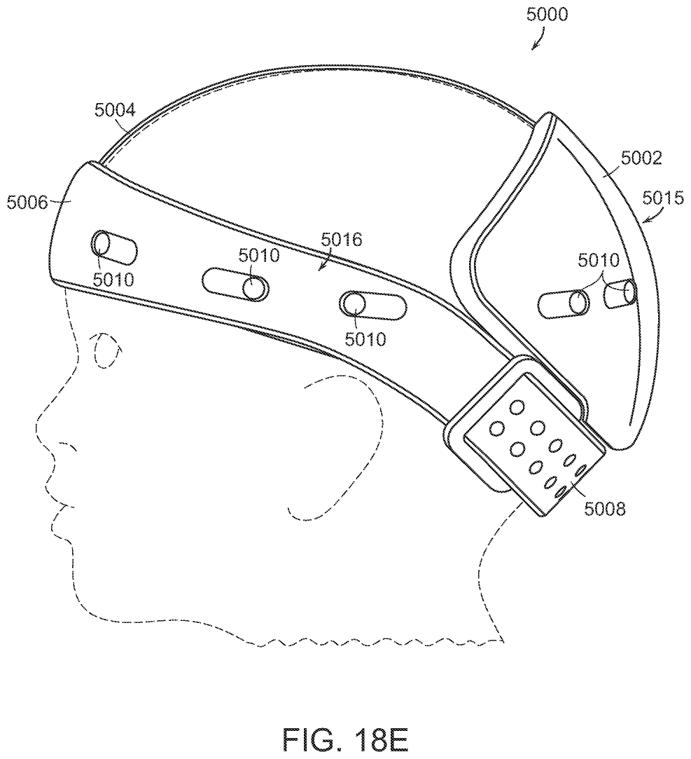

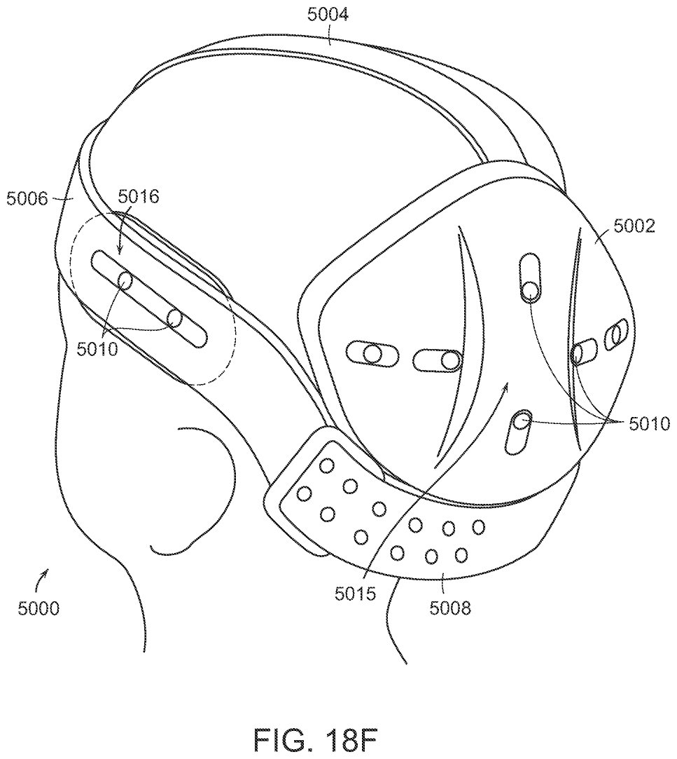

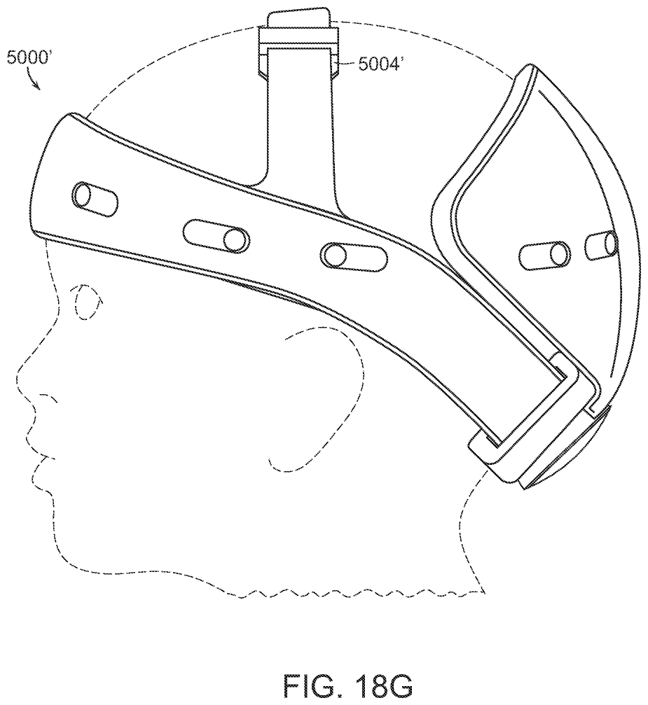

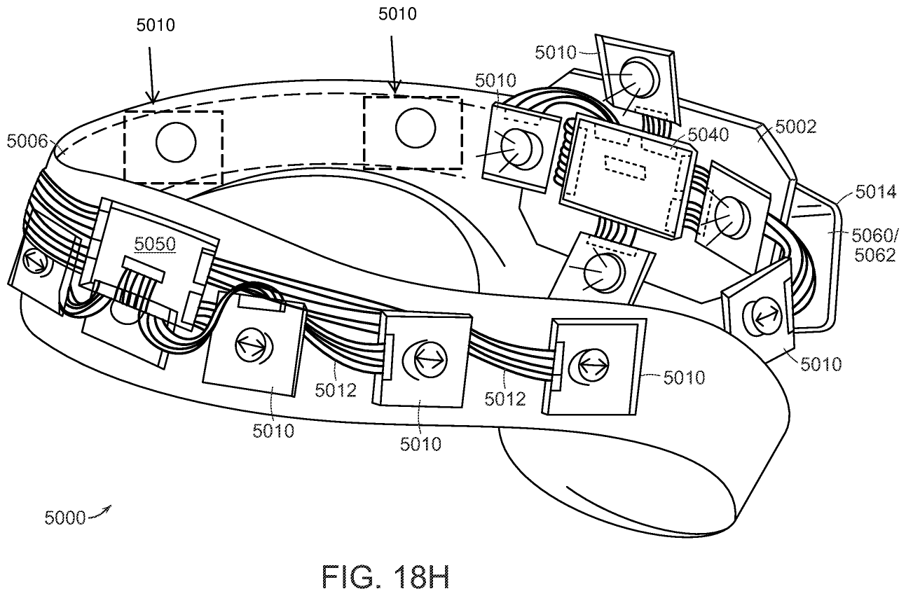

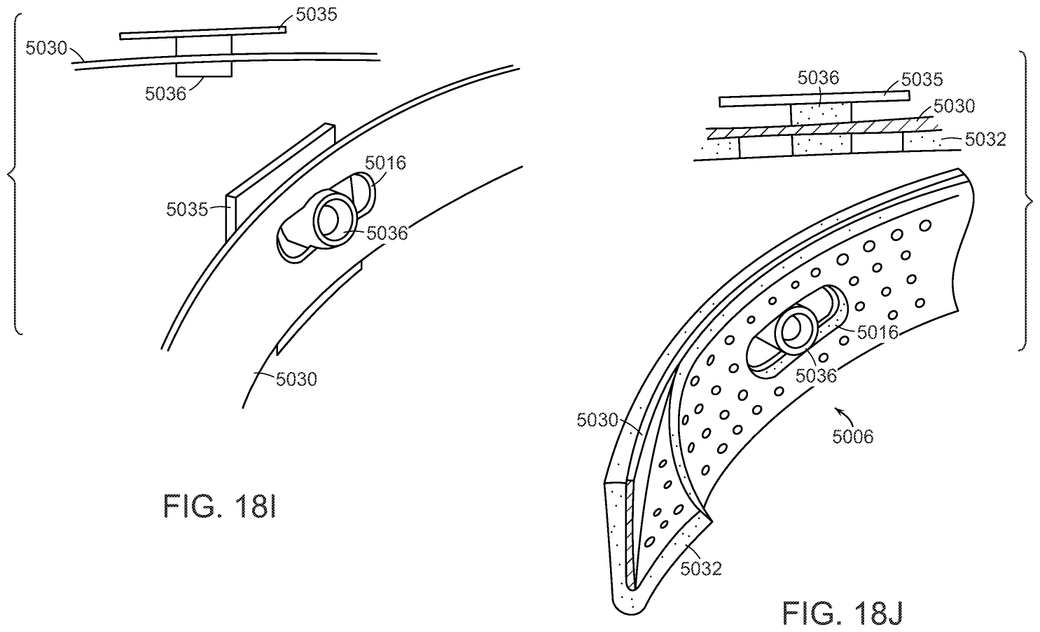

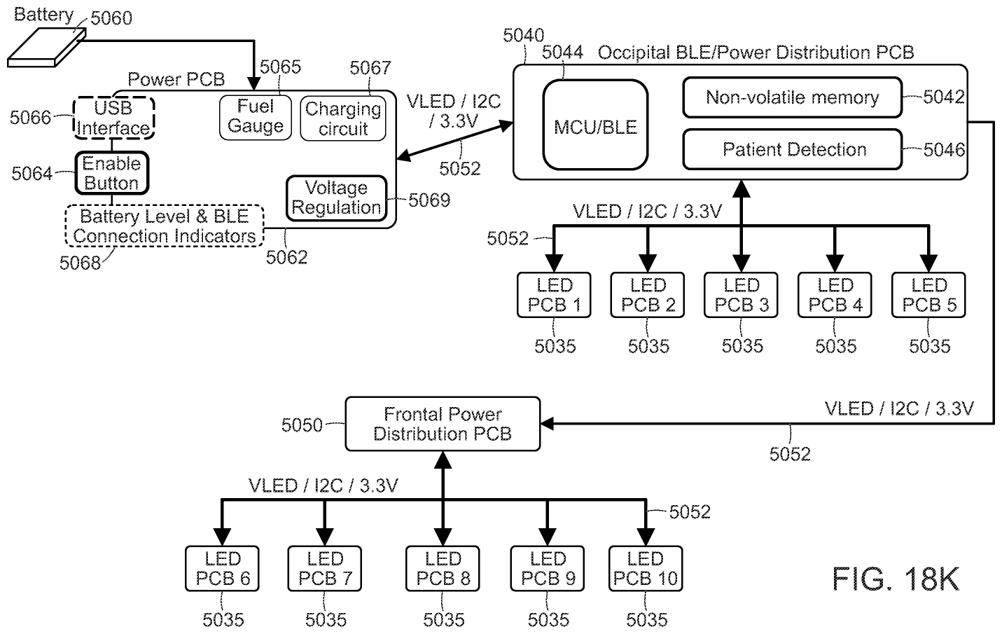

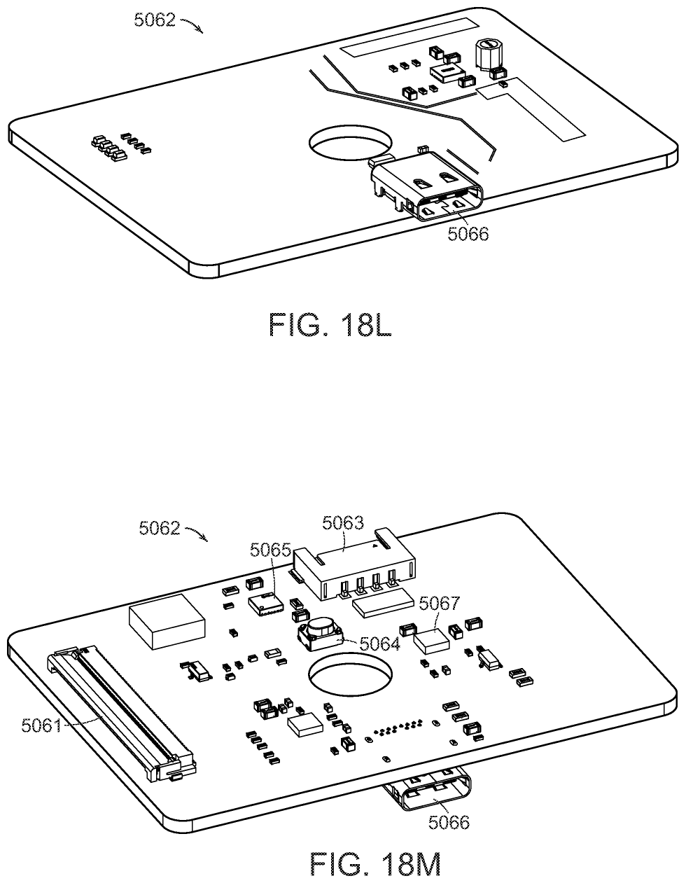

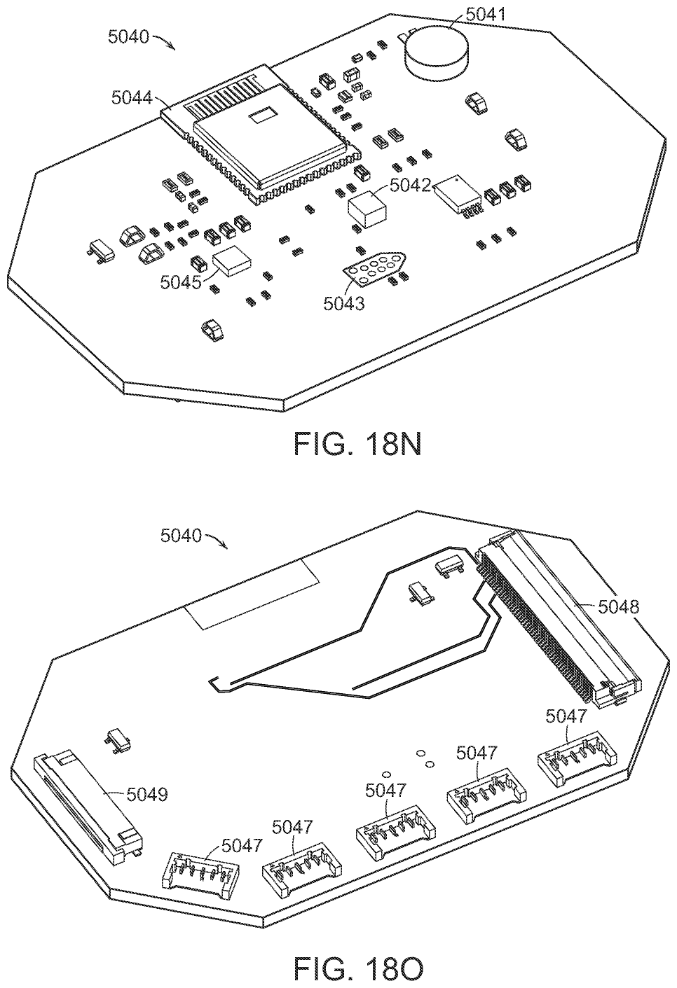

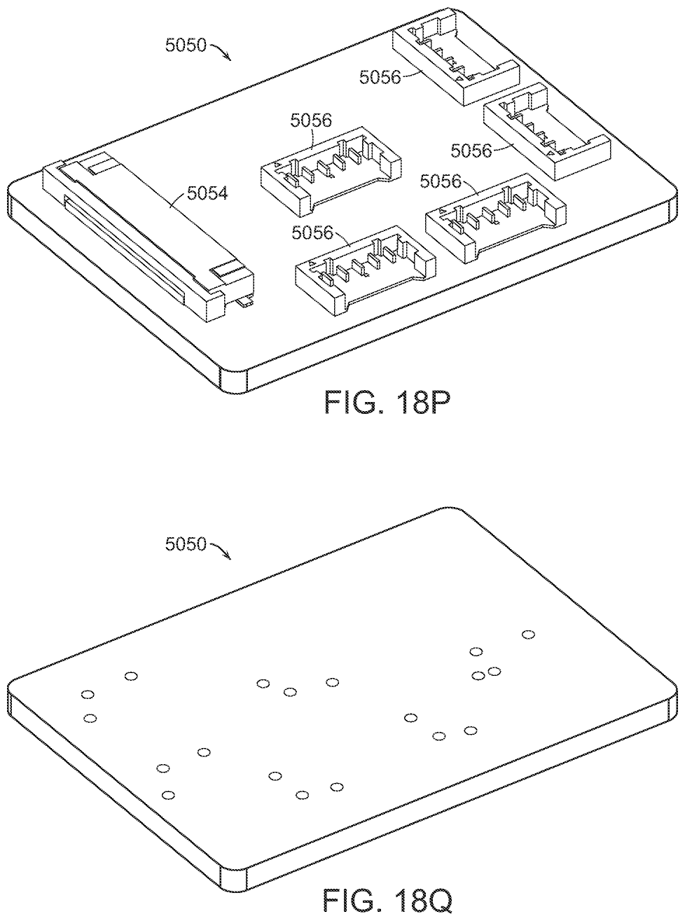

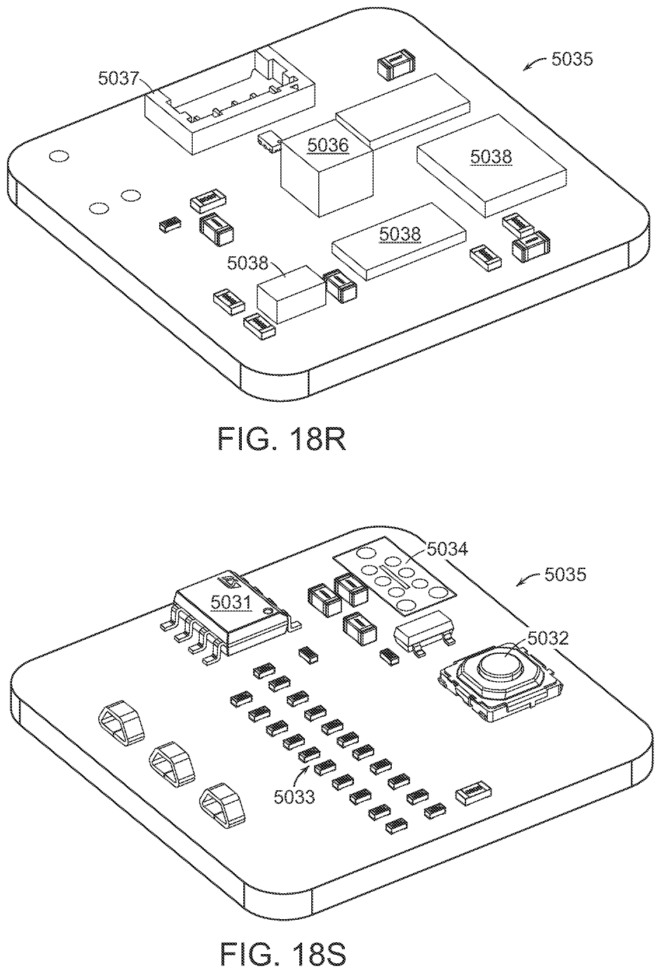

Shown in FIG. 18A is a further implementation in which the head wearable device 800 has light emitting devices 810 at spaced locations around the head of the patient connected by a cable 812 to a circuit housing having a first portion with an on/off switch 802 and a second portion with one or more control buttons or actuators 804 to manually select operating modes of the device as described herein. Headphone speakers and/or microphones 814 can be mounted to the head worn device 800 or speakers/microphones can alternatively be within a first tablet 820 that can be used by the patient during a therapy session. The first tablet or mobile phone 820 can be connected by wire or cable 806 to device 800 and can emit sounds or auditory signals for improving linguistic skills of the patient as described herein. The display on the first tablet can also be used to display images or video to the patient during the therapy session. A second tablet or mobile phone 840 can also communicate with the head worn device 800 and/or the first tablet by a cable or wireless connection 808. Tablet 840 can be used by an operating user to control operation of one or both of the head worn device 800 and first tablet 820, before, during or after a therapy session. For example, if an EEG sensor is used during a therapy session, this can serve to monitor the procedure or calibrate the power level to be used on a particular patient to establish the minimum level therapeutic dose, and optionally to also set a maximum dose for each period of illumination during the session, and further optionally to select which regions of the brain of the patient are to be illuminated during a session. The first tablet may be programmed only to provide the auditory and/or visual components to the patient, whereas the second tablet can be programmed solely for use by the operator or clinician to manage the therapy provided to one or more patients in separate sessions. The tablet used to manage patient data can also be connected by wired or wireless connection directly to an external EEG processing station 156 that receives wireless transmission of digitized EEG signals from the headset.

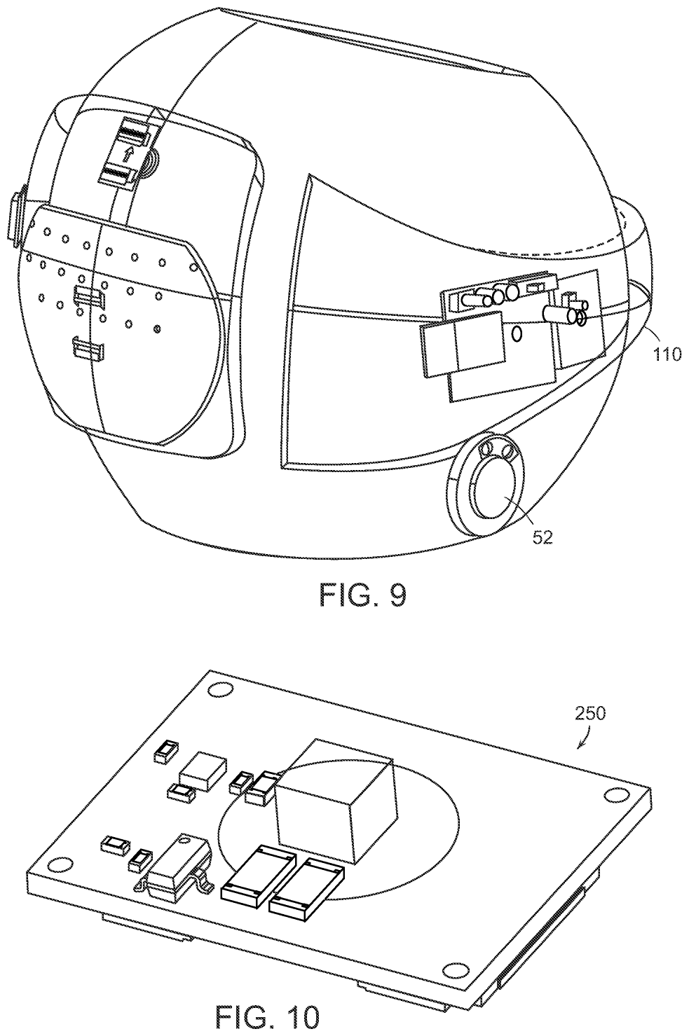

Shown in FIG. 18B is a top view of a headset 850 that incorporates an EEG electrode array including EEG electrodes 855, 856 located at different locations around the head of the patient. As described in further detail below such an EEG sensor array can be integrated with a light emitter array positioned around the head of the patient at different separate locations, or partially or entirely collocated with the EEG electrodes. The separation between light emitters and EEG electrodes can be adjusted depending on the treatment protocol for different neurological disorders as described herein. Light emitters and/or electrodes can be mounted on bands 854 that extend towards an upper housing or top portion 852 which can have a crown shaped bottom surface that can conform to the top of a user's head to help stabilize the housing 852 which preferably has a low profile shape with a light weight. The bands 854 can extend to a circumferential portion of the headset 880 extending around the user's head such as depicted in FIG. 18A and other figures shown and described herein. The bands 854 or tubes containing the necessary wiring for EEG electrodes and/or light emitters can be situated on all sides of the user's head so as to enable placement of light emitters and/or EEG electrodes as required for a specific application. Between 8-64 or more EEG electrodes can be mounted on the headset along with the same or a different number of light emitters as described herein. The tubes or bands 854 can also extend from the rear electronics module 882 for embodiments in which there is no housing 852 on the top of the patient's head such as depicted in connection with FIGS. 2A-3 and 9-12, in which case the EEG circuitry can be integrated into module 882. The tubes or bands can include connectors 857 at one end so that they can be easily removed and replaced. Such a system can thereby incorporate disposable components thereby allowing the electronics module to be reused with other patients without loss of sterile conditions. In a further embodiment, the housing can be configured to include circuitry for detecting EEG signals wherein wiring from the EEG electrodes 855, 856 is amplified with amplifiers 858 for each channel followed by analog to digital converter 860 for each channel, processing of the digital signals with processor 862 that can multiplex the signals for transmission by wireless transmitter 864 and antenna 866 that communicates with an external transceiver as described herein. A power source such as a battery 869 and an impedence excitation source 865 can also be located in the housing 852. The circuitry and one or more power sources in housing 852 can also be located in a second circuit housing 882 situated on the back of the patient's head. The housing 882 can also include circuitry, power and control operations for the light emitter system as previously described herein. A further embodiment can employ a battery situated in the rear housing to power both the circuitry in the top housing 852 and the rear housing 882. In a further embodiment, the circuitry in both housings can utilized a single processing unit to manage digital signals for both digital circuits. Such a control processor can be configured to control the light emitters, and the sensed EEG digital signals for external transmission. The transmitter 864 and antenna 866 can thereby operate as a transceiver to manage receipt of control signals to control operation of the light emitters and also control transmission of digitized EEG data. The EEG and light emitter control and processing functions can be performed by one or more processors. For applications requiring a larger number of EEG electrodes and/or light emitters one or more control and processing functions can be performed by a field programmable gate array (FPGA) or by an application specific integrated circuit (ASIC) configured to process the larger number of channels at faster speeds and at lower power levels. Such a configuration can further reduce the size and weight to accommodate use by pediatric patients. If a larger number of light emitters and/or EEG sensors is required, the electronic components required to operate the integrated system can also be mounted on a single flexible circuit board extending from the rear housing to the top housing within a single flexible sleeve. Note that the bands 854 can comprise flexible or semi-rigid plastic components. The bands can comprise tubes or prongs in which the wiring for the EEG electrodes or light emitters can extend. The light emitters can comprise LEDs or laser diodes, for example, that can be mounted in the ends of the tubes that are oriented to direct light through the cranium. A lens can be attached to the distal exit aperture of the light emitter to define the focal region of tissue within the cranium. Where an array of light emitters is used, the distal lenses can provide overlapping illumination volumes of tissue. The tubes can comprise polyethylene or polypropylene materials that can be sterilized or replaced after a single use. As seen in FIG. 18D, the EEG electrodes and/or light emitters 874, 876, 878 can each be spring loaded with springs 872 to cause the contact surfaces to press against the scalp. Thus, the tissue contact surfaces 877 can move relative to the housing along axis 875. Arrays of two or more EEG electrodes, light sensors and/or light emitters can be housed 870 at well-defined separation distances to provide repeatable measurements. Each housing 870 can be situated on a band at selected locations on the head so as to precisely locate the sensors and light emitters as described herein to transmit and receive signals through the cranium and also through the lambdoid suture and/or the squamosal suture. In pediatric patients these small lines between cranial plates have less density and are thus more transmissive of red and infrared light signals for photobiomodulation as described herein. X-ray images of these sutures lines can be obtained for each patient in which it is desirable to direct illuminating light through one or more suture locations. This enables precise positioning of the light emitters relative to the suture lines. In such applications, the headset must be properly secured to the head to align the light emitters to the suture lines for the therapeutic period.

FIG. 18E illustrates a side view of a head wearable device 5000 in accordance with some embodiments described herein. The head wearable device 5000 includes a head mounting frame or headband 5006 connected to an occipital mount 5002. The frame or headband 5006 can comprise a length of material that surrounds the head of the user wherein light sources are mounted to direct light inward through the cranium. The head mounted material has an inner surface and an outer surface on which circuit components can be mounted. The headband 5006 can include a size adjustment mechanism 5008. Alternatively, the optoelectronic circuit components described herein can be mounted on a flexible head wearable fabric with sufficient elasticity to be worn on different head sizes, but exert sufficient tension to cause the light emitting surface of the LEDs to come into contact with the scalp as previously described. Such features are described previously in the present application and can be adapted for this preferred circuit design. Note that this system can also include communication, user interface, audio and visual systems previously described generally in the present application. LED modules 5010 are mounted at locations on the headband 5006 and the occipital mount 5002 to illuminate different portions of a user's cranium with therapeutic light. The head wearable device 5000 can include a head strap or band 5004 that connects a frontal portion of the headband 5006 to the occipital mount 5002 at the back of the user's head.

The head wearable device 5000 is formed at least partially of a soft material with airy open spaces in some embodiments. In some embodiments, a surface of the headband 5006 is formed of a non-porous material to improve sterilizability and cleanability. In some embodiments, the material can include one or more of low-density polyethylene (LDPE), silicone, or ethylene-vinyl acetate (EVA) closed-cell foam. In some embodiments, the headband 5006 can include light, pastel, or bright colors that appeal to children for pediatric therapy applications.

The size adjustment mechanism 5008 can enable adjustment of the headband 5004 for comfort and/or to improve contact or coupling between the user's scalp and the LED modules 5010. The size adjustment mechanism 5008 can include a band or strap that tightens against a patient's skull or tightens below the occipital bone of the skull. In some embodiments, the adjustment mechanism can include a fastener such as a hook-and-loop fastener. For example, the headband 5006 can include two separated straps that fasten with the hook-and-loop fasteners or a single strap that passes through a retaining ring and doubles back upon itself so that hooks on the end of the strap can attach to a separate portion of the strap that includes loops. In some embodiments, the adjustment mechanism can include a snap closure wherein snaps or pegs in one portion of the headband 5006 connected with a variety of snaps or holes at different positions along the headband 5006. Similarly, the head strap 5004 can include a size adjustment mechanism to enable sizing adjustments of the head strap 5004 to improve comfort for a user with a given head size. The same size adjustment mechanisms 5008 described herein for the headband 5006 can be employed in the size adjustment mechanism for the head strap 5004.

FIG. 18F illustrates a rear view of the head wearable device 5000 illustrating the occipital mount 5002. The occipital mount 5002 can include one or more LED modules 5010. The LED modules 5010 can be spaced in a pattern in some embodiments such as a cross pattern or a polygonal patterns such as square-shaped or diamond-shaped. In some embodiments, the LED modules 5010 can be positionable at different positions on the headband 5006 or occipital mount 5002. For example, headband 5006 or occipital mount 5002 can include multiple receptacles at different locations so that the LED modules 5010 can be moved to different receptacles as needed. In some embodiments, the headband 5006 or occipital mount 5002 can include racetracks 5016 that allow the LED modules 5010 to translate or slide in one or more directions to improve positioning of the LED module 5010.

In some embodiments, the occipital mount 5002 can include an electronics housing 5014 to accommodate a battery or other electronics or power sources to power elements of the head mounted device 5000 such as the LED modules 5010.

FIG. 18G illustrates an alternative embodiment of the head wearable device 5000′ with different placement of the head strap 5004′ in accordance with some embodiments described herein. The head wearable device 5000′ is substantially identical to the head wearable device 5000 described above except that the head strap 5004′ extends from one lateral side of the headband 5006 to the other lateral side of the headband 5006. This differs from head strap 5004 as shown in FIGS. 18E-F that extends from the occipital mount 5002 forward to a portion of the headband 5006 adjacent to the patient's forehead. In some embodiments, the head strap 5004, 5004′ is omitted from the head wearable device 5000, 5000′ entirely.

FIG. 18H illustrates the head wearable device 5000 according to various embodiments described herein. This figure illustrates a patient-contacting surface of the occipital mount 5002 to show arrangement of LED modules 5010 and mounting of the occipital power distribution printed circuit board (PCB) 5040. In this embodiment, the occipital power distribution PCB 5040 controls power distribution to a group of five LED modules 5010 arranged as a group in the occipital region of the patient's brain. A separate frontal power distribution PCB 5050 is positioned at the forehead region of the headband 5006 and powers a group of five LED modules 5010. Each group of LED modules 5010 is connected to a respective PCB 5050 by connection wires 5012. In some embodiments, the connection wires 5012 pass directly from the respective PCB 5040, 5050 to the corresponding LED module 5010 as opposed to passing serially through multiple LED modules 5010. In this arrangement, direct powering and addressing of each LED module 5010 by the PCB 5040, 5050 is possible as there is no daisy chaining. This enables consistent power delivery to all LEDs on the head mounted device 5000.

FIGS. 18I and 18J illustrate placement of an LED module in a multi-material headband 5006 in accordance with some embodiments described herein. The headband 5006 can include a relatively stiffer core 5030 surrounded by a relatively softer foam liner 5032 in some embodiments. The core 5030 can retain an LED 5036 of an LED module 5010 in a stable position within an opening 5016 (such as an oval or racetrack opening) due to friction fitting between the LED 5036 and the core 5030. The foam liner 5032 can surround the stiffer core 5030 to provide a comfortable surface against the patient's head. As shown in the perspective and end views of FIG. 18I, the LED module 5010 can include the LED 5036 and LED PCB 5035, which is described in greater detail below. The LED 5036 projects from the LED PCB 5035 and extends through the core 5030. As shown in FIG. 18J, a front surface of the LED 5036 can be flush with the surface of the foam layer 5032 that contacts the patient so that the LED is placed as close as possible to the patient's scalp without projecting outward to form a painful pressure point.

FIG. 18K schematically illustrates the electrical connections between elements of the head wearable device in accordance with several embodiments described herein. The electrical system of the head wearable device 5000 includes a battery 5060, the power PCB 5062, the occipital power distribution PCB 5040 (sometimes abbreviated as the occipital PCB), and the frontal power distribution PCB 5050 (sometimes abbreviated as the frontal PCB). The power PCB 5062 is connected to the occipital PCB 5040 via a cable 5052. The cable 5052 can carry signals for multiple operations or functionalities simultaneously. In some cases, the cable 5052 can carry signals at different voltages. In various embodiments, the cable 5052 can transfer power to the LED PCBs 5035 at the appropriate voltage VLD, can carry voltage for logic circuits such as TTL at 5 V or 3.3V, or carry voltage for inter-integrated circuit (I₂C) bus or Pulse-Width Modulation (PWM) Limit applications. Similar cables 5052 with similar functionalities connect the occipital PCB 5040 to the frontal PCB 5050; connect the occipital PCB 5040 to LED PCBs 5035; and connect the frontal PCB 5050 to LED PCBs 5035.

The power PCB 5062 can include a power gauge integrated circuit (IC) 5065, a charging circuit 5067, a voltage regulation module 5069, a USB interface 5066, an enable button 5064, and battery level and BlueTooth® low energy (BLE) connection indicators 5063. The power gauge IC 5065 can monitor the voltage level of the battery 5060 to determine the remaining energy (power) in the battery 5060. The battery level indicators 5068 can indicate visually to the user the level of remaining energy in the battery 5060 as measured by the power gauge IC 5065. The charging circuit 5067 enables wireless charging of the battery 5060 using inductive charging techniques such as those that conform to the Qi® wireless charging standard or other charging standards. The voltage regulation module 5069 can regulate the output voltage provided on the cable 5052 to the other components. The enable button 5064 can include a mechanical or electrical switch operable by the user to turn on or off the electrical systems of the head mounted device 5000. The USB interface 5066 enables wired charging of the battery 5060 and/or provides an interface for re-programming (e.g., flashing) or debugging components of the power PCB or connected PCBs. The USB interface 5066 can also be used for transfer of data such as usage statistics (e.g., recorded or sensed power levels, up-time or down-time, or error statuses).

The occipital PCB 5040 includes a microcontroller unit (MCU) and BlueTooth® low energy (BLE) module 5044, non-volatile memory 5042, and a patient detection module 5046. The MCU/BLE module 5044 can control power output to each individual LED PCB 5035. The MCU/BLE module 5044 enables communication with external devices using the BLE protocol. For example, an external device such as a computer, tablet, or smartphone operated by the user can wirelessly send instructions to the MCU/BLE control module 5044 to adjust individual LEDs to different power settings over time according to a therapeutic program. The memory 5042 can include one or more of logical address information or instructions to control the LED PCBs 5035. The patient detection module 5046 can detect whether or not the head mounted device 5000 is being worn by the patient. If the patient detection module 546 detects that the head mounted device 5000 is not being worn by a patient, it can send signals to the controller 5044 or the power PCB 5062 to disable power to the LEDs to prevent light output. This automatic shutoff when the patient is not present can conserve power in the battery 5060 and provide safety by preventing illumination from being turned when the light could accidentally enter a patient's eyes, for example. This is especially important in pediatric applications where a child may inadvertently remove the head mounted device 5000 and could accidentally aim the light at their eyes if it were not automatically shut off. In some embodiments, the patient detection module 5046 can operate by employing sensors that detect whether LED light is being reflected by a very close object. Alternatively, the patient detection module 5046 can use an accelerometer or inertial position system to determine the orientation of the head mounted device 5000 and disable the device when the position is not consistent with placement on a head of a sitting or standing individual.

FIGS. 18L and 18M illustrate respective top and bottom views of the power PCB 5062 in accordance with some embodiments described herein. The USB interface port 5066 is located on a top side of the power PCB 5062. The power gauge IC 5065, charging circuit 5067, and enable button 5064 are located on a bottom side of the power PCB 5062. The power PCB 5062 can include a connector 5063 to connect the cable from the battery 5060. The power PCB 5062 also includes a connector 5061 to connect the cable 5052 to the occipital PCB 5040.

FIGS. 18N and 18O illustrate respective top and bottom views of the occipital PCB 5040 in accordance with some embodiments described herein. The occipital PCB 5040 can include a battery 5041 to power an on-board real-time clock (RTC) that clocks operations of the device, a motion sensor such as an accelerometer 5045 for headgear orientation detection, an electronically erasable programmable read-only memory (EEPROM) 5042 or other non-volatile memory, the control module 5044, and a BLE control module programming port 5043 on a top side of the PCB 5040. The BLE programming port 5043 enables debugging or reprogramming of the control module 5044. The EEPROM memory 5042 can also be accessed and erased through the port 5043 or through signals sent from the power PCB 5062 through port 5066. The memory 5042 can include instructions for controlling LEDs in a particular program or pattern. The system can operate a first plurality of light sources according to a first pattern and control a second plurality of light sources (LEDs) according to a second pattern. Different light sources can emit at different optical wavelengths and at different duty cycles for example. The circuitry can include one or more current level sensors or temperature sensors to control operation of the device. This can include closed loop control of the light sources, for example, to maintain optical output from each light source within 5-10 percent of the nominal output to treat a selected condition of the patient for the prescribed therapeutic period. It is also important to prevent operating temperatures of the device so as to prevent thermal injury to the patient. Thus, the head mounted device is configured to automatically shut off the light sources and/or power if such a condition is detected. The accelerometer 5045 can send signals to the patient detection module 5046 to help detect whether the system is in a ready state for being mounted on the patient's head. The accelerometer or other sensor (pressure sensor, light sensor, etc as described herein) can also be configured to sense a change in orientation of the head mounted device relative to the users' head and to transmit a signal to the control module to shut off the LEDs. The system can also operate a state machine that is regularly updated with operational data that can be automatically transmitted to an individual that is monitoring the therapeutic session. An alarm signal can also be sent to a remote user communicating that at least a portion of the system has been disrupted or changed so that remedial action can be taken to continue or stop the therapy session. The system clock can report the time elapsed, the time remaining or the time of disruption.

The occipital PCB 5040 includes individual connectors 5047 to connect cables 5052 to the LED PCBs 5035. The occipital PCB 5040 also includes a connector 5049 to connect cables 5052 to the frontal PCB 5046 and a connector 5046 to connect the cables 5052 to the power PCB 5062. Any of the connectors 5049, 5047, 5048 can include a flat-flex connector that allows low-profile and flexible connections to reduce the space taken by cables 5052 within the headband 5006 or occipital mount 5002.

FIGS. 18P and 18Q illustrate respective top and bottom views of the frontal PCB 5050 in accordance with some embodiments described herein. The frontal PCB 5050 includes individual connectors 5056 to each LED PCB 5035 in the frontal or forehead region of the headband 5006. The frontal PCB 5050 also includes a connector 5054 to the cables 5052 from the occipital PCB 5040. The connector 5054 can be a flat-flex connector. Note that the use of two or more power control circuits mounted on the same or separate circuit boards can be used to control different sets of light sources. This can provide greater control over the operating conditions of the two or more groups of light sources to maintain operation with nominal operating conditions. Thus, a first plurality of light sources can be operated by a first power control circuit and a second plurality of light sources can be controlled by a second power control circuit. The use of a constant current control circuit improves the safe operation of the system. This system enables operation of the system at 500 milliamps and at a duty cycle of 35%, for example. It is desirable to operate the system at a currents above 400 milliamps and a duty cycle of less than 45% to improve safety, efficacy and durability of the system. This design is scalable, so that a third power control circuit can be used to control a third plurality of light sources, etc. Thus, a design of the system for older children or adult use can integrate more light sources to illuminate larger areas of the brain. In such a system 15-20 or more LEDs can be integrated for optimal control for a battery operated system, or for applications in which a power cable can be used to provide power the head mounted system.

FIGS. 18R and 18S illustrate respective top and bottom views of the LED PCB 5035 in accordance with some embodiments described herein. The LED PCB 5035 can include a second controller 5031 with LED current feedback, an LED driving circuit 5038, the LED 5036, a temperature sensor, and an LED unique identification (ID) circuit 5033. The LED PCB 5035 can also include a connector 5037 to connect via cable 5052 with either the occipital PCB 5040 or the frontal PCB 5050. The LED driving circuit 5038 can be an LED constant current circuitry that maintains the proper current output to drive the LED 5035. The temperature sensor can sense the temperature and send signals to the microcontroller 5031. The microcontroller 5031 can halt power to the LED 5036 if an over-temperature or overheating condition is detected. The LED unique ID circuit 5033 can include a resistor bank that is set differently for each LED PCB 5035 in the system. The occipital PCB 5040 or frontal PCB 5050 can use the LED unique ID circuit 5033 to identify each connected LED PCB 5035 upon connection or at a subsequent time. The LED PCB 5035 can include a microcontroller reset switch 5032 that is not operator accessible but can be used during initial setup or repair. The LED PCB 5035 also can include a controller programming port 5034 to enable debugging or reprogramming of the LED PCB 5035.

Photobiomodulation can be used to treat several ailments including Alzheimer's disease, post-traumatic stress disorder (“PTSD”), cognitive enhancement, cognitive impairment from trauma and/or injury, depression, anxiety, mood disorders, Parkinson's Disease, strokes, Global Ischema, and Autism Spectrum Disorder (“ASD”). In particular, these ailments can be treated with transcranial photobiomodulation, which involves targeted light energy to the brain. The devices associated with performing transcranial photobiomodulation are often applied over the head, such as in certain embodiments described herein. However many such devices, can be cumbersome and in particular, for especially sensitive patients (e.g., children with ASD), it can be difficult to comfortably apply the device for treatment over a meaningful duration of time without the patients attempting to shift or remove the device.

Aspects of the disclosed technology include devices for photobiomodulation, which can be used to treat various patients including ASD children and older adults. Consistent with the disclosed embodiments, the photobiomodulation device may be sized and shaped to fit inside the oral cavity of the human mouth. The photobiomodulation device may include one or more light emitting diode (LED) lights, which may be located in a center portion of the device. Further, the LED emitters may be positioned to point downwards or to other regions, such that light from the device affects blood vessels that flow within the body to regions of the brain. Preferred embodiments of can be used in conjunction with methods and devices that can illuminate blood vessels within the brain or that supply blood directly to the brain such as the internal carotid artery. Further, the LED light emitters may emit light at one or more wavelengths which can be red, infrared, and/or a combination of the two. The LED light emitters may have a material (e.g., latex, silicone, rubber, etc.) surrounding it that allows the light to penetrate tissue within the mouth yet is also difficult to chew. The surrounding material can comprise one or more lenses to couple the emitted light onto the tissue that contacts a surface of the device and wherein the tissue contains regions of vascular flow that is illuminated with the device. The photobiomodulation device may further include an extendable portion that protrudes outwards from the device in a longitudinal direction. In some examples, the extendable portion may include the LED light emitters. The photobiomodulation device may be shaped similarly or substantially similar to a pacifier, for example. Therefore, a wearer of the photobiomodulation device can bite down or suck on the extendable portion while it is inside the mouth. The photobiomodulation device may further include one or more processors, transceivers, or power sources (e.g., batteries). Preferred embodiments can also include a cavity to collect a sample of fluid from within the mouth for further testing and analysis, such as a saliva sample. A surface of the device, or the cavity, can optionally include a sensor to measure further characteristics of the tissue and/or the sample. The sensor can be electronically connected to circuitry for readout of sensor data during use. The sensor can include a light sensor such as a photodetector to measure light from the tissue and/or sample. The device can be configured to communicate with an external portable communication device as previously described herein to store patient data in a memory, and to further process and communicate data as described in the present application.

In some examples, the frequency and/or type of light emitted by the photobiomodulation device may be adjustable. Therefore, the photobiomodulation device may further include a controller that allows the user to adjust the frequency, illumination pattern and/or intensity of light. Also, in some examples, the photobiomodulation device may be paired to a user device (e.g., via Bluetooth®) that can send instructions to adjust the operating parameters of light emitted. In some examples, the position of the LED light emitter may be adjustable, i.e., the LED light emitters can be moved or scanned in another direction (e.g., left, right, up, or down).

Some implementations of the disclosed technology will be described more fully with reference to the accompanying drawing. This disclosed technology can be embodied in many different forms, however, and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein can include, but are not limited to, for example, components developed after development of the disclosed technology.

It is also to be understood that the mention of one or more method steps does not imply that the methods steps must be performed in a particular order or preclude the presence of additional method steps or intervening method steps between the steps expressly identified.

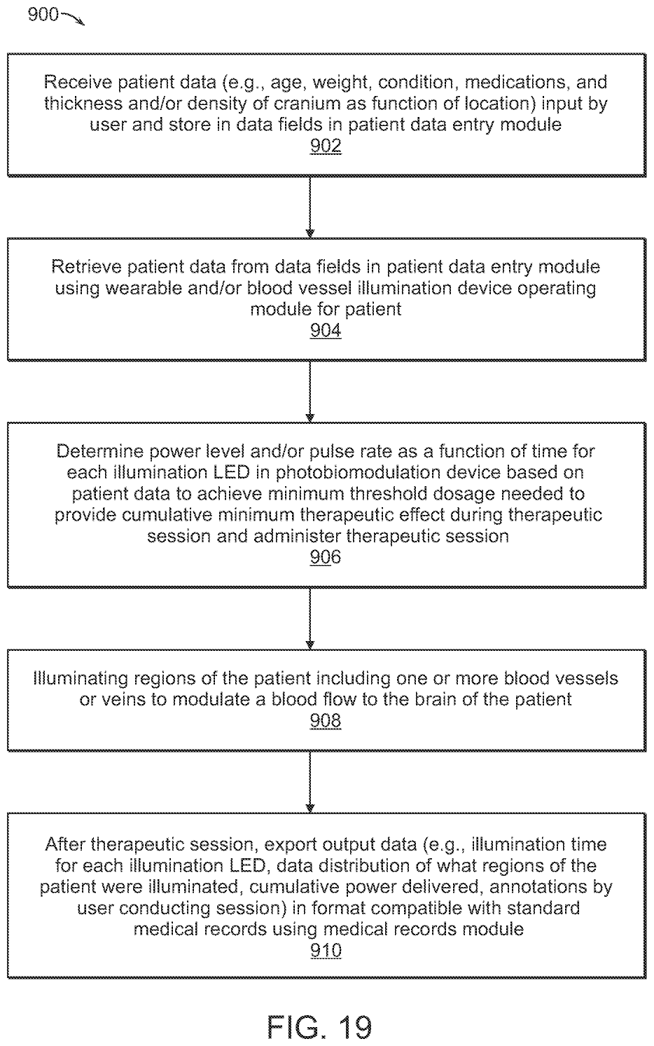

Shown in FIG. 19 is a process sequence 900 that can be implemented with a controller on the therapeutic device or in conjunction with an external controller as described herein. The user interface is configured to receive and store patient data 902. Certain data can be retrieved manually or automatically 904 so that parameters for a therapeutic session as implemented 906 on the PBM device. The device is actuating to illuminate vascular tissue of the patient 908 to thereby modulate blood flow within the body including the brain of the patient. This can be implemented in combination with transcranial illumination of brain tissue in selected patients, which can include transcranial illumination of blood vessels in proximity to brain tissue that is also receiving light. A record of the therapeutic session is than communicated 910 for storage and further analysis.

Further methods of the invention can include photobiomodulation of lymphatic vessels to improve drainage to treat neurological conditions. See, for example, the publication by Semyachkina-Glushkovskaya et al., “Photobiomodulation of lymphatic drainage and clearance; perspective strategy for augmentation of meningeal lymphatic functions”, Biomedical Optics Express, Vol. 11, No. 2, February 2020, the entire contents of which is incorporated herein by reference. By using PBM to augment the rate of drainage of lymphatic fluid from the brain there are improvements in transport of components that adversely impact neurological condition of the patient. Improved drainage of the lymphatic system has been shown to improve the condition of autistic patients. See Antonucci et al., “Manual Lymphatic Drainage in Autism Treatment”, Madridge Journal of Immunology, Vol. 3, Issue 1, December 2018, the entire contents of which is incorporated herein by reference. Thus, methods of treatment can include transcranial PBM of lymphatic channels in the brain. The LED array elements can be actuated to illuminate lymphatic channels at the energy densities described herein to perform therapeutic treatment of the patient. Imaging technologies including Optical Coherence Tomography (OCT) and ultrasound have been used to monitor lymphatic flow as well as blood flow and perfusion.

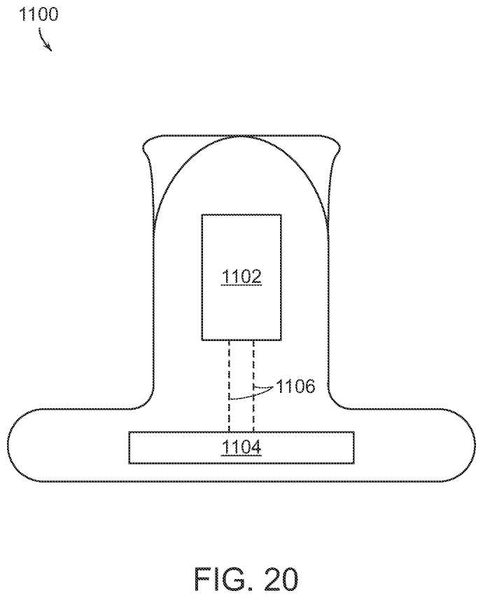

FIG. 20 shows an example photobiomodulation device 1100. As shown in FIG. 20, in some implementations the photobiomodulation device 1100 may include LED light emitters 1102, one or more circuit boards including circuitry 1104 powered by one or more batteries, and/or internal wires 1106 connected to light emitters 1102, among other things including sensors or other components as described herein. The photobiomodulation device circuitry 1104 can further include one or more processors, a transceiver, and/or a controller. The photobiomodulation device 1100 may be paired with a user device (e.g., smartphone, smartwatch, or tablet device as described herein), which may provide instructions that may determine a frequency of transmitted light and/or the type of light (e.g., red light or infrared light) pattern or intensity distribution.

Using the photobiomodulation device 1100, certain methods of the present disclosure may perform photobiomodulation (stimulating brain with light). In some examples, photobiomodulation may be performed simultaneously with linguistic training to treat, for example, children with ASD as described previously herein. Preferred photobiomodulation devices and methods may include several near infrared and/or red light emitters to stimulate certain blood vessels within the cranium or that flow directly into the brain. Methods associated with the photobiomodulation device 1100 may include determining a position the LED lights 1102 to output the infrared and/or red lights. The light absorbed by the blood vessels may increase the production of ATP, which may provide the neurons more energy to communicate with each other and provide increased brain connectedness. In some examples, the photobiomodulation device 1100 can determine an amount of change in ATP. Further, the photobiomodulation device 1100 may continue to output light until a desired amount of ATP change is reached. Therefore, in some examples, the photobiomodulation device 1100 may further include one or more sensors that can determine the amount of ATP of cells within a predetermined distance of the device. Also, using a controller, the frequency of transmitted light and/or the type of light emitted by the photobiomodulation device 1100 can be manually or automatically adjusted.

Methods for providing photobiomodulation may include determining the light frequency, location of the LED lights (e.g., blood vessels needing increased ATP), whether ATP production increased, and the overall effect of the treatments. Accordingly, based on the determined overall effect on the brain, the photobiomodulation device 1100 may be dynamically adjusted on a user-specific basis.

The photobiomodulation device 1100 may be specifically tailored for children and/or older adults, such that it alleviates certain ailments (e.g., ASD, Alzheimer's Disease). Further, the photobiomodulation device 1100 may be used on a daily basis, in the convenience of the family's home, without a need for a specially trained therapist. Moreover, the photobiomodulation device 1100 can be non-invasive, not require a prescription, and lack side effects.

Methods of the present disclosure may further include determining the location(s) of the light diodes that may be used to stimulate specific brain areas responsible for language, comprehension, energy production, and/or for self-regulation (e.g., reducing anxiety). Therefore, application of light therapy by the photobiomodulation device 1100 may result in improved sleep, improved language, and/or improved general cognition. The methods may also include determining total power, power density, pulsing, and/or frequency. The total power may be 400-600 mW (0.4-0.6 W) with 100-150 mW per each of four panels.

Further, the photobiomodulation device 1100 may be comprised of a comfortable material for prospective patients. For example, the photobiomodulation device 1100 may be comprised of plastic, latex, silicone, rubber, and/or the like. Because ASD patients in particular are especially sensitive, the aforementioned shape and materials may be integral in allowing ASD patients to wear it for a sufficient amount of time without being irritable. Of course, the photobiomodulation device 1100 is preferably both safe and comfortable. The electric components (e.g., processors, wires, transceivers, etc.) may be included within the interior of the photobiomodulation device 1100 and may be difficult to reach by children, for example. Further, the weight of the photobiomodulation device 1100 may be light enough to allow it to be held in the mouth comfortably. Moreover, the photobiomodulation device 1100 may require a power source (e.g., batteries) that allows it to be portable. The emitter section 1102 can include one or more sensors as described herein to measure a fluid analyte, such as glucose or lactose, in a saliva sample that can be captured by a small port into a cell within the device. A motion sensor or piezoelectric sensor can be included to measure mechanical movements of the device.

As mentioned above, the photobiomodulation device 1100 can be paired to a user device, such that a user can adjust the position of the LED light emitters 1102 as well as the frequency and/or type of light. Further, the photobiomodulation device 1100 may be dynamically adjustable based on the determined ATP levels of the cells near photobiomodulation device 1100 before and after application of the light treatment. For example, the photobiomodulation device 1100 can be configured to treat a predetermined amount of ATP that the cells near the photobiomodulation device 1100 can have. Then, the photobiomodulation device 1100 may determine an amount of the ATP cells before application of the light treatment and during the application of the light treatment. Based on the determined ATP levels, the photobiomodulation device 1100 may continue to apply light treatment until the predetermined amount of ATP is reached.

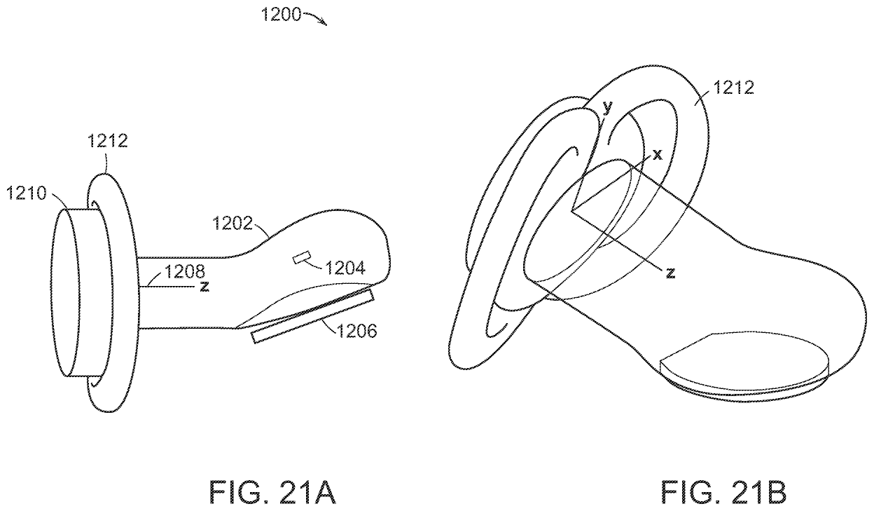

Shown in the side and perspective views of FIGS. 21A and 21B, respectively, is a device 1200 for partial insertion within the oral cavity. In this example, a pacifier such as used with infant children can be used for PBM therapy. The child can grasp the elements 1212 that serve as a mouthguard so as to limit the insertion portion or distal region of the device to a predetermined length. This defines the portion of tissue in the mouth, such as on the tongue 1206, that is illuminated by LED 1204, which is at a fixed position within the insertion portion 1202 so as to illuminate tissue region 1206. A wire 1208 can extend from circuit housing 1210 at the proximal portion of the device to connect to the LED emitter 1204. The distal section 1202 is shaped to improve contact with tissue region 1206 when placed in the patient's mouth.

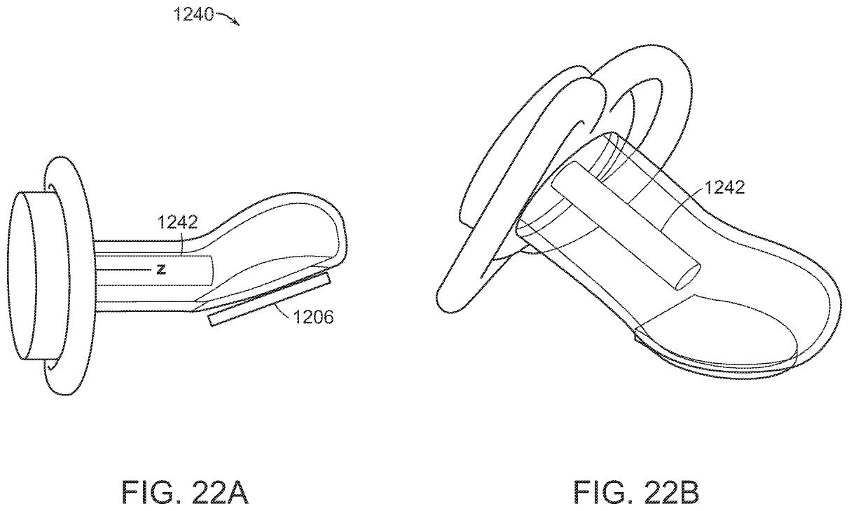

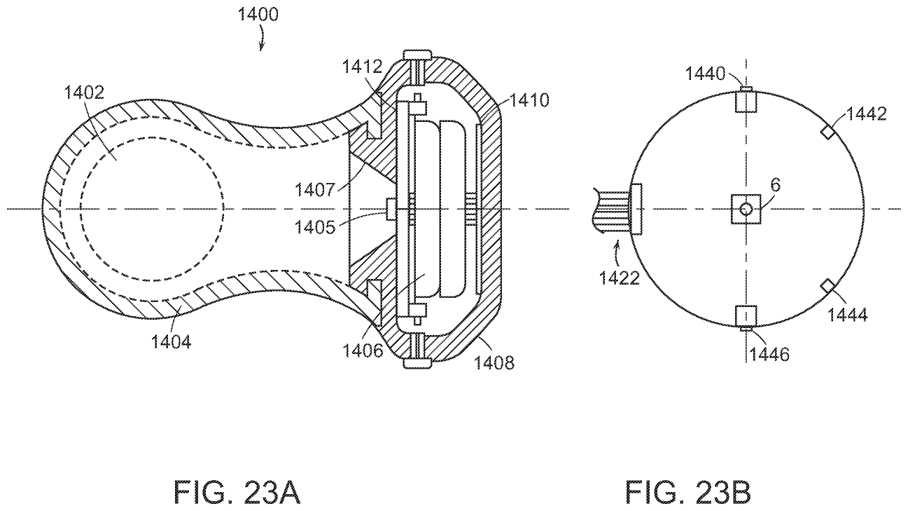

FIGS. 22A and 22B show side and perspective views of a further embodiment wherein the LED emitters are mounted to a circuit board in the circuit housing adjacent to a tube or optical fiber coupling 1242 that extends into the distal section to optically couple the LED emitted light onto the region 1206. The material of the distal section can optionally include reflector elements or surfaces to improve coupling onto tissue 1206 that can include one or more blood vessels to be illuminated. The insertion portion can be encapsulated in a white diffuse coating or layer that more efficiently couples light onto a clear portion 1402 (see FIG. 23A) of the surface configured to transmit light onto the tissue 1206.

Shown in FIGS. 23A and 23B are cross-sectional and end views respectively showing a circuit board 1406 within the circuit housing 1408 wherein the LED is mounted on a distally facing portion of the circuit board 1406. A frame 1412 situates connections within the housing 1408 to buttons or actuators 1440, 1446 that enable the user to control operation of the device including on/off operation and control of operating parameters. Indicator lights 1442, 1444 can indicate the operating status of the device. A cable 1422 can also be connected to the device to provide battery recharging, communication and control functions. The insertion portion 1404 can be attached to housing 1408 around reflector surfaces around the LED emitter that emits light onto the output surface 1402 that contacts the tissue surface.

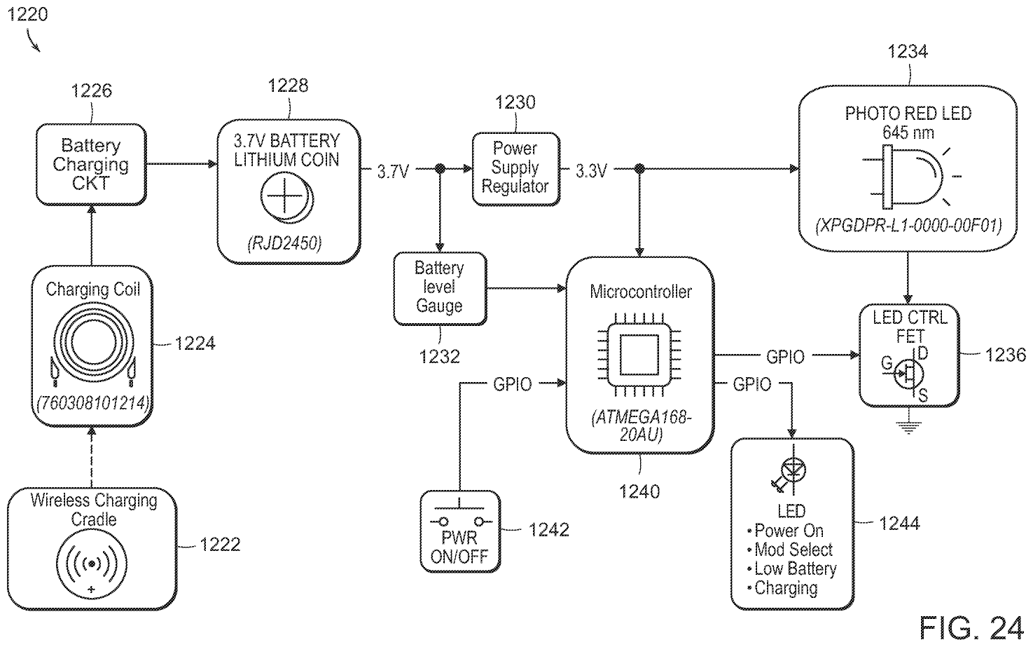

FIG. 24 illustrates an exemplary circuit 1220 for operating the PBM device. This embodiment can include a wireless charging element 1222 connected to a charging coil 1224 that enables charging circuit 1226 to charge the battery 1228 which can be a 3.7 V lithium battery in this example. The circuitry can be mounted on a circuit board in which a processor such as microcontroller 1240 is connected to a battery gauge 1232, power supply regulator 1230. LED control field effect transistor 1236 and LED 1234. The device can include an on/off switch 1242 and an LED status indicator light 1244.

Fetal Alcohol Syndrome (FACS) results from a baby being exposed to alcohol during the neonatal stage of development. The fetal liver cannot metabolize alcohol (ethanol), so when alcohol enters the blood stream of the developing baby it interferes with the delivery of nutrition and oxygen to the developing organs. Therefore, it interferes with cell growth and proliferation. Specifically, ethanol in the developing baby's blood stream can result in permanent and irreversible brain damage. Neurological and behavioral symptoms often reflect the affected brain areas. The most common affected brain areas are the prefrontal cortex, which results in difficulties with focus, decision making and social interactions; the hippocampus can also be affected, which results in difficulties with forming memories; the cerebellum, which results in difficulties controlling movements; and also the corpus callosum, which affects overall brain function and results in mental retardation.

Brain imaging studies have specifically identified these areas (frontal lobe, corpus callosum, hippocampus and cerebellum) as being most likely to be affected by FACS. Other imaging studies showed that FACS results in poor communication between various brain areas (i.e., poor brain connectivity). Children affected by FACS usually have smaller brains. In addition, children affected by FACS may develop physical characteristics like microcephaly, growth retardation, dislocated limbs, certain facial features (e.g., thinner upper lip) and cardiological problems. It should be noted that the physiological features of FACS may or may not be present and a percentage of FACS children are misdiagnosed as having ADHD (due to their difficulties with focus, organization, planning, decision making and memories). It should also be noted that Fetal Alcohol Syndrome disproportionally affects babies in the minority communities (specifically in the Black community). No treatment is currently available for FACS.

tPBM (stimulation of the brain with near-infra red light) has been shown in animal and human studies (in vivo and in vitro) to increase blood oxygenation, cerebral blood flow, and mitochondrial ATP production. In addition, EEG and NIRS data has shown that tPBM improves brain connectivity. Therefore, blood brings more oxygen and nutrition to the brain. In addition, increased ATP production results in more neurogenesis and synaptogenesis. Furthermore, functional brain connectivity has been shown to improve after one session. tPBM has been shown to be beneficial for traumatic brain injury, depression, ischemic stroke, and Parkinson's disorder. In addition, it has been shown to be effective for Down syndrome, autism and ADHD. Similarly, tPBM can be effective for the neurological symptoms of FACS by increasing the amount of oxygen and nutrients delivered to the brain, improving functional brain connectivity, and increasing neurogenesis and synaptogenesis. Specifically, the effect may be most pronounced in cortical structures (frontal lobes), which improves organization, focus, and decision making. The effect on memory and motor functions may be less pronounced since sub-cortical structures are implicated (e.g., hippocampus and cerebellum). However, due to neuroplasticity, the beneficial effect of tPBM may be most pronounced when treatment is administered to young children.

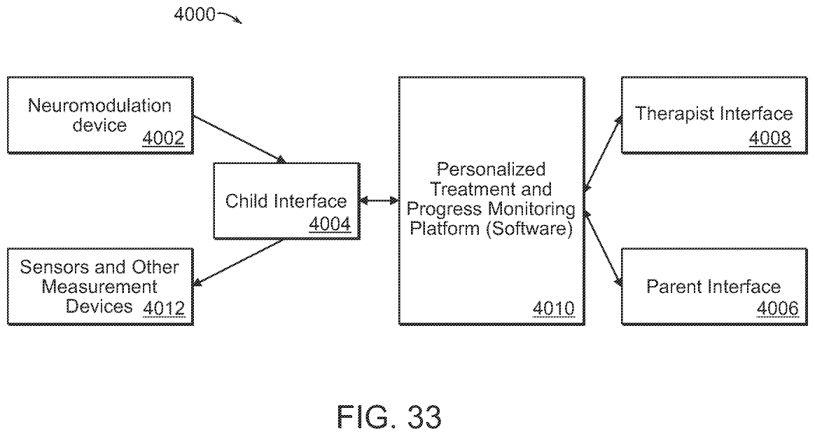

The preferred embodiment of the present invention includes a neuro-biomodulation device, sensors and other measurement instruments, a software platform for personalized treatment recommendation and progress monitoring, and respective child, parent, and therapist interfaces described herein and as shown in FIG. 33.

The User Profile Module (UPM) 3002 receives all data related to the child's profile, including demographic data, neuro-developmental assessment data, health data, ongoing device data, treatment history, progress indicators, parental assessments, and behavioral data. The user profile is continuously updated with treatment and progress data and contains both baseline and longitudinal data.

The Reference Population Module (RPM) 3016 is the database containing all user profiles created within the UPM and is used as a calibration and testing sample for the Machine Learning Module 3018.

The neuro-developmental assessment module (NDA) 3006 uses the user profile together with questionnaire data to assess the baseline and continuous performance of the child along attachment, playing, communication and language, and other behavioral factors using a range of metrics and scores the child's current state for each of the measures. As treatments are administered, the NDA scoring is updated and resulting recommendations modified. The NDA assessment together with the UPM data feed into the Personalized Treatment Module (PTM).

The Personalized Treatment Module (PTM) 3004 leverages the cluster-treatment mapping data from the Machine Learning Module 3018 to create personalized plans for the Neuromodulation Treatment Module (NMT) 3008 and the Cognitive Programming Module (CPM) 3010. This includes physical device treatment duration, intensity, and frequency as well as specific cognitive treatment activity portfolios to be administered to the child.

The Neuromodulation Treatment Module (NMT) 3008 leverages the personalized treatment recommendations of the PTM and provides them across the parent and therapist interfaces for administration.

The Cognitive Programming Module (CPM) 3010 leverages the personalized treatment recommendations from the PTM and provides cognitive activity and treatment content to the child via the child interface and/or the parent/therapist interfaces.

The Sensor and Quantitative Data Feedback Module (SQD) 3012 captures data from physical sensors and devices such as EEG, heart rate and pulse wearables, and other devices alongside with performance data of the child on the cognitive programming module (CPM) as well as parental and therapist feedback to measure the impact of the treatments on the NDA metrics of the child.

The Performance Progress Module (PPM) 3014 compares the individual data from the SQD 3012 with expected progress thresholds established for the selected cluster within the RPM 3016 and provides effectiveness scores for administered treatments.

The Machine Learning Module (MLM) 3018 uses an embedding-based vectorization methodology to create user profile vectors that are then mapped into different profile-treatment clusters which match an individual profile background to treatments that have the highest effectiveness scores for individuals with similar user profile vectors.

The Feature Selection and Vectorization Module (FSV) 3020 takes individual data fields from the UPM and vectorizes them into n-dimensional numeric feature vectors that represent the initial UPM data. The FSV 3020 uses a modified tf/idf in the form of a boolean (feature) frequency-inverse boolean vector frequency (BF/IBVF) measure to convert different measurement variables into numeric data indices of a vector. This vectorization of variables can be expressed as:

$$BF ( {b}_{l} , {d}_{j} ) = \frac{Freq ( {b}_{l} , {d}_{j} )}{{\sum }_{l} Freq ( {b}_{l} , {d}_{j} )}$$

$$IDBF ( {b}_{l} , {d}_{j} ) = log \frac{{N}_{doc}}{{N}_{doc} ( {b}_{l} )}$$

$$BFIDBF = BF ( {b}_{l} , {d}_{j} ) * IDBF ( {b}_{l} , {d}_{j} )$$

Wherein bₗ represents the l^{th} bin, dⱼ represents the j^{th} document, Freq(bₗ, dⱼ) is the number of times bin l appears in document j, ΣₗFreq(bₗ, dⱼ) represents the total number of bins in document j, N_{doc} is the number of users, and N_{doc}(bₗ) is the number of uses with the bin bₗ.

The FSV 3020 then engages in dimensionality reduction of the vector into a lower-dimensional orthogonal subspace that captures as much of the variation of the UPM and RPM data sets as possible.

The Embedded Cluster Predictor (ECP) 3024 takes the reduced dimensionality vectors from the FSV 3020 and clusters them based on the K-means or LDA, bypassing BF/IBVF and dimensionality reduction) into bi-partite profile-treatment clusters using effectiveness scores from the PPM as a omni-distance measure.

The Deep Learning Module (DLM) 3022 takes data from the SQM and from the PPM for the reference population and adjusts the ECP 3024 clusters continuously with new feedback data from all users. The raw SQM data is augmented with gaussian noise to reduce inter-subject variability. DNN learns features automatically. Further details describing the use of machine learning computational systems and methods, and particularly with respect to the application of neural networks to EEG data and similar data sets is described in Moinnereau et al., “Classification of Auditory Stimuli from EEG with a regulated recurrent neural network reservoir”, arXiv:1804.10322v1 [eess.SP], published 27 Apr. 2018, the entire contents of which is incorporated herein by reference. A regulated recurrent neural network (RNN) to characterize hearing of patients to auditory stimuli or speech which improved on the classification rates over other methods such as a naïve Bayes classifier or support vector machine (SVM). In this method, EEG signals recorded by methods previously described herein, are transformed into spike trains that are accumulated in a reservoir of connected neurons. For example, the process of encoding of signals into spike trains can assume that an analog signal is the result of filtering spike trans with a reconstruction finite impulse response (FIR) filter and uses the FIR to find spikes in the EEG signals according to the following two equations at every given time τ.

$${e}_{1} ( τ ) = {\sum }_{k = 0}^{M} \vert s ( k + τ ) - h ( k ) \vert$$

$${e}_{2} ( τ ) = {\sum }_{k = 0}^{M} \vert s ( k + τ ) \vert$$

where s represents the different EEG signals, h(k) is the reconstruction FIR filter, and M is the order of the filter. This can digitize the output to enable further processing of the data. The readout from the reservoir is classified over time using linear regression. The results of the RNN were compared to a deep neural network (DNN) which can employ, in this example, three convolutional layers where EEG signals were input into the network. This enables EEG measurements to be used to measure the response of system users to auditory stimuli as described above where photobiomodulation is used to treat patients to improve language learning. Thus, changes in language comprehension can be quantified over time during treatment.

In a further example, as described in Chambon et. al., “A deep learning architecture for temporal sleep stage classification using multivariate and multimodal time series,” IEEE Neural Systems of Rehabilitation Eng. 26, 758-69 (2017) involving the use of a multivariate time series to classify sleep patterns, and further machine learning techniques to classify and score sleep patterns using a convolutional neural network in Chambon et. al., “A deep learning architecture to detect events in EEG signals during sleep”, arXiv:1807.05981v1 [eess.SP] 11 Jul. 2018, in which the learning problem can be solved by a minimization problem to detect events from EEG signals measured during sleep. This can be expressed as event detector \hat{f}

\hat{f}∈ar [symbol] min [symbol] _{x∈X}[l(E(x)),f(x)]

where an iterative computational process is used to detect events during sleep. As the patient is treated with photobiomodulation therapy, changes in the detected events can be quantified and stored over time in relation to the course of treatment. Note that x denotes input EEG signals while E(x) denotes a “true” or measured event; there are also “default” events used to train the neural network. An event label l can have a zero-value or a selected non-zero value such as 1.

The process detects spindles and K-complexes jointly or severally to detect and score events during sleep. Thus, the network identifies classes of sleep events using EEG sensor data that can be measured during the course of photobiomodulation therapy to characterize and quantify therapeutic outcomes of the treatment.

The architecture of the Deep Neural Network (DNN) contains convolutional neural network (CNN) layers to extract frequency domain features and recurrent neural network (RNN) layers to capture the temporal structure. Thus, the DNN generates quantitative frequency domain and time domain data that are used to characterize the results of the photobiomodulation therapy and can be used to guide modifications of the therapeutic plan for the patient and serve to train the network to treat subsequent patients that are within the same class in the PPM so that the appropriate thresholds are established.

The Cluster-Treatment Mapper (CTM) 3026 takes the individual's UPM vector and maps it into the clusters identified in the ECP to identify the optimal treatment options based on the RPM. It then feeds the identified cluster into the PTM for further processing.

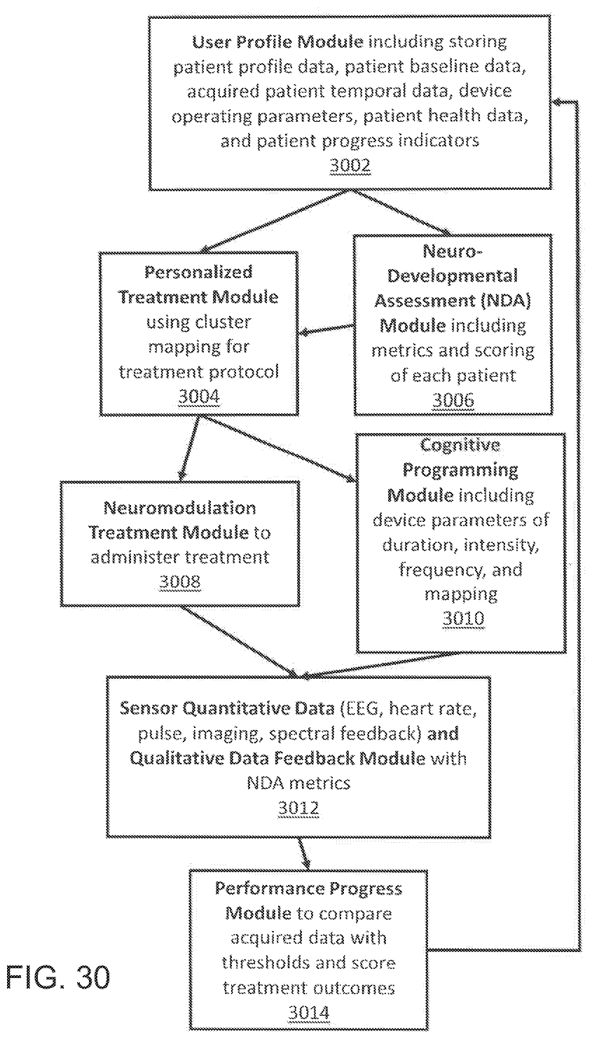

The entire process for an individual is captured in the flowchart in FIG. 30. User's data is captured by the UPM 3002, and an neuro-developmental assessment is performed 3006. The PTM 3004 leverages the MLM 3018 and the RPM 3016 to identify ideal treatments using the NMT 3008 and CPM 3010 modules. Once the user engages in the treatment, the SQD 3012 module records data on the effect of the treatment, and the PPM 3014 assesses the effectiveness of the treatment, recording all the activities back into the UPM 3002.

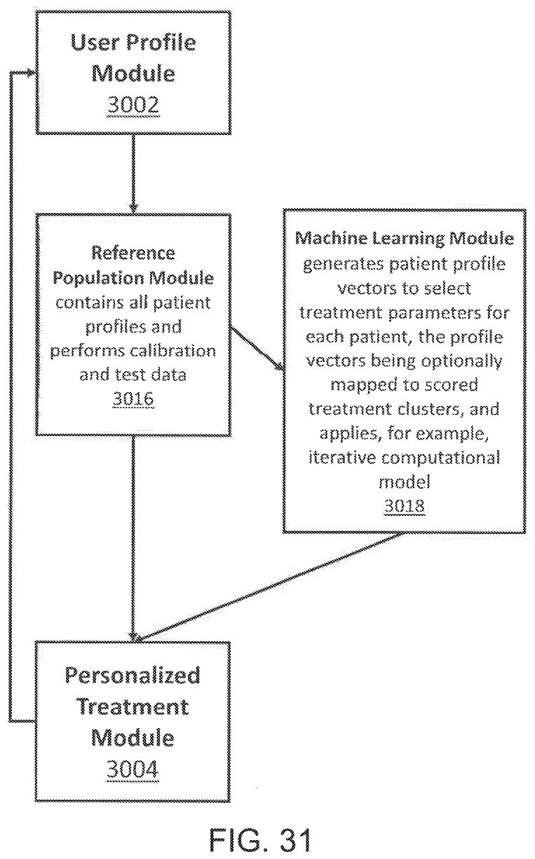

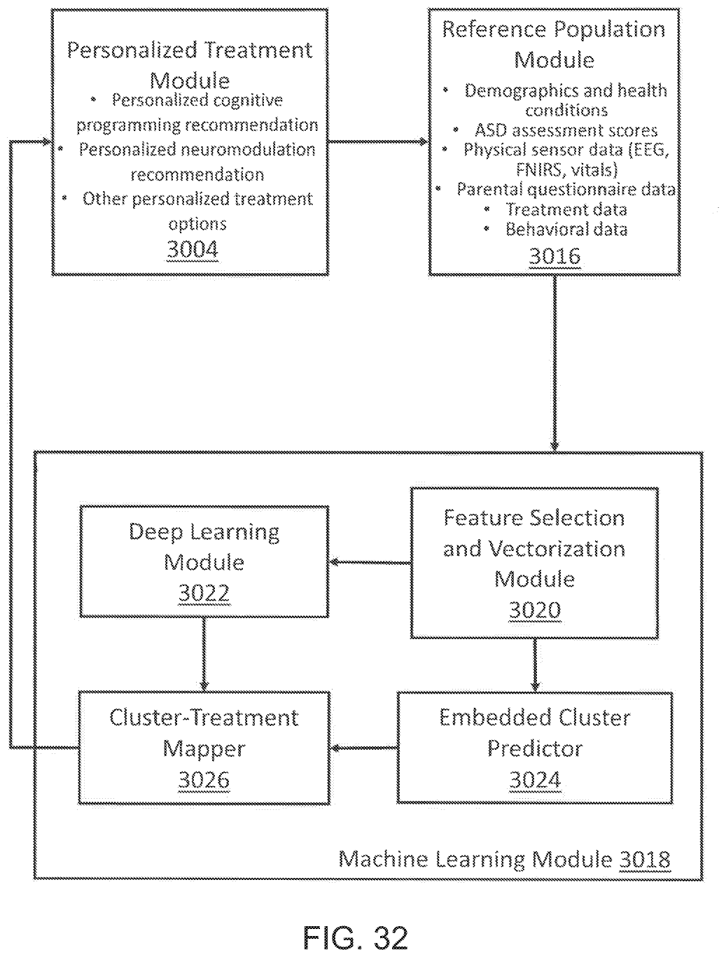

The reference population treatment cluster analysis process and the respective personalized treatment mapping process are shown in FIGS. 31 and 32 respectively. These constitute the integration of feedback into the system for learning and furthering the personalization prediction accuracy.

In FIG. 33, a preferred embodiment of the system 4000 is shown with its components, including the neuro-biomodulation device 4002 as described previously herein, physical and quantitative data collection systems 4012, interfaces for the user (child 4004 such as a tablet), parents (using a personal computer 4006 to access a website interface), and therapist interface 4008 and the software system 4010 for personalized treatment needs, assessment, recommendation, and progress monitoring. The neurobiomodulation device 4002 can include the head-mounted photobiomodulation device described previously and configured to be worn by a child during a therapeutic session.

In some embodiments, the photobiomodulation and/or neurobiomodulation devices and methods of use described herein can produce statistically significant improvements in autism symptoms and related indicators. A clinical trial was conducted with the objective of demonstrating that transcranial photobiomodulation (tPBM) is an effective treatment modality to improve language and communication skills in children with ASD. In recent pilot studies, tPBM has been shown to be an effective treatment for certain conditions such as stroke, traumatic brain injury (TBI), and depression (Ando et al., 2011; Cassano et al., 2018; Naeser et al., 2020). Pilot studies have shown that tPBM can reduce symptoms of autism (Ceranouglu et al, 2019; Leisman et al 2018). The above referenced study hypothesized that children with ASD will demonstrate improvement in communication skills and language acquisition with experimental treatment.

The present clinical study examined the effect of tPBM modulation on symptoms of autism in children 2-6 years old. It is a randomized, placebo-controlled, double-blind study. Twenty-nine participants were enrolled and wore the tPBM device (such as the photobiomodulation device(s) of the present disclosure) for 6 minutes, and in which illuminating light delivering an energy in a range of 16-24 joules was administered during each session. Each participant completed 16 sessions during an 8-week course of the study. Data about children's behavior was collected from parents through weekly interviews. Children's therapists are interviewed regarding any observed changes in child's behavior. Before and after treatment scores of Childhood Autism Rating Scales are compared for placebo and experimental conditions. EEG measurements from frontal, occipital and temporal area were collected before and after each treatment.

The results of the trial indicated a statistically significant reduction in autism symptoms as measured using the Childhood Autism Rating Scale (CARS). A CARS assessment was made for each participating child (n=21) before the beginning of the trial and after the trial by a blinded researcher. The CARS is defined such that higher scores indicate worse autism symptoms. As shown in the tables below, treatment using photobiomodulation in accordance with the systems and methods described herein produces a statistically significant improvement in children in the “experimental” group whereas children in the “placebo” group did not have a statistically significant improvement. In the preliminary CARS results Control: before: 40.3 7.5 after 39.8 7.3 Experiment: before: 45.3 5.7 after 35.4 4.7 T-test p-value: 1-sided: p=0.001; 2-sided: p=0.005.

| control # | Original CARS | Final CARS | diff |
|---|---|---|---|
| 10 | 40.6 | 40.7 | 0.1 |
| st. dev. | 7.2 | 7.4 | 7.7 |

| experiment | Original | Final | diff |
|---|---|---|---|
| 13 | 45.3 | 35.3 | −10.0 |
| st. dev. | 5.7 | 4.7 | 3.4 |

| excluding 2 pre- | | | |
|---|---|---|---|
| clinical experiment | Original | Final | diff |
| 11 | 44.8 | 34.5 | −10.3 |
| | 6.0 | 4.6 | 3.6 |

| | with | | excluding | |
|---|---|---|---|---|
| | pre- | | pre- | |
| | clinical | | clinical | |
| T-Test | 0.231% | 0.116% | 0.196% | 0.098% |
| DiD | 10.1 | | 10.4 | |

In the final clinical study results, the CARS scoring showed a statistically significant decrease for overall based on 16 active patients and a slight decrease in the placebo control group of 14 patients:

| experiment | Original | Final | diff |
|---|---|---|---|
| 16 | 43.1 | 34.6 | −8.5 |
| | 5.4 | 4.8 | 4.1 |

| control | Original | Final | diff |
|---|---|---|---|
| 14 | 40.5 | 39.5 | −1.0 |
| | 6.8 | 8.1 | 8.1 |

| | with pre- | | excluding | |
|---|---|---|---|---|
| | clinical | | pre-clinical | |
| T-Test | 0.015026 | 0.007513 | 0.015595 | 0.007798 |
| DiD | 7.6 | | 7.6 | |

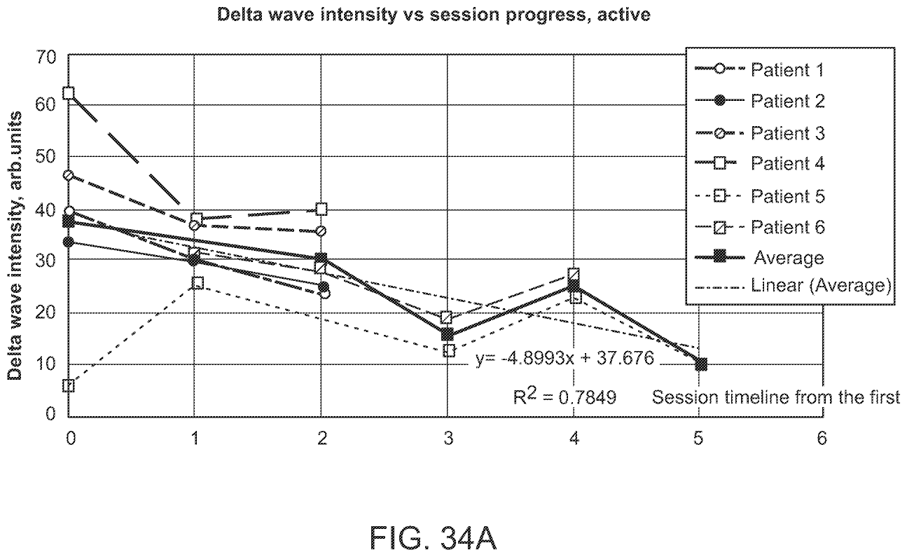

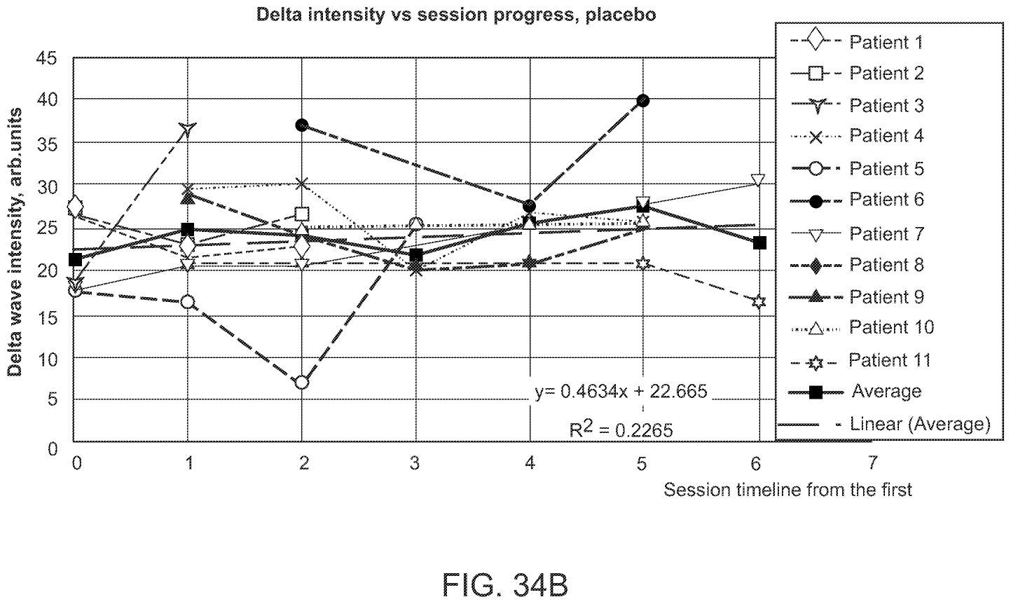

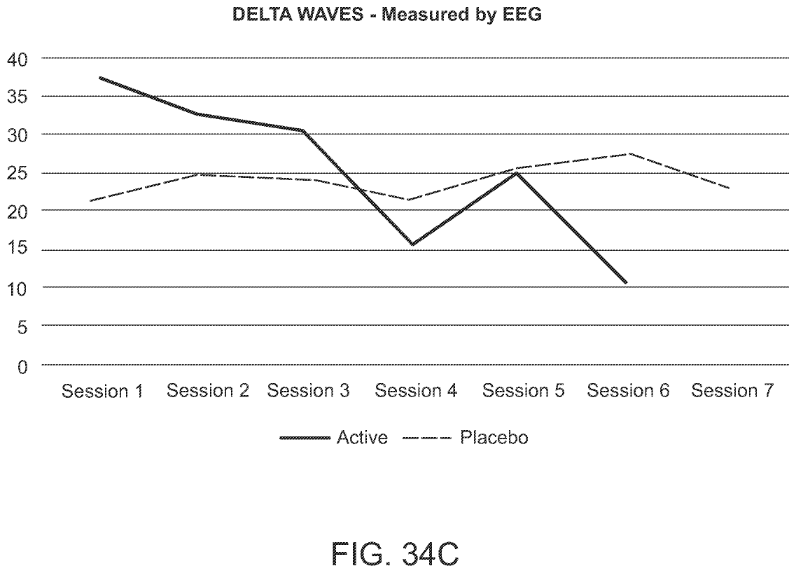

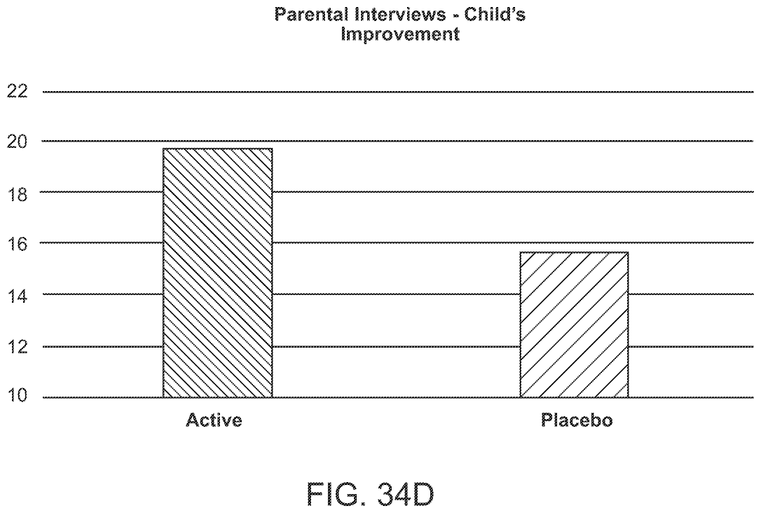

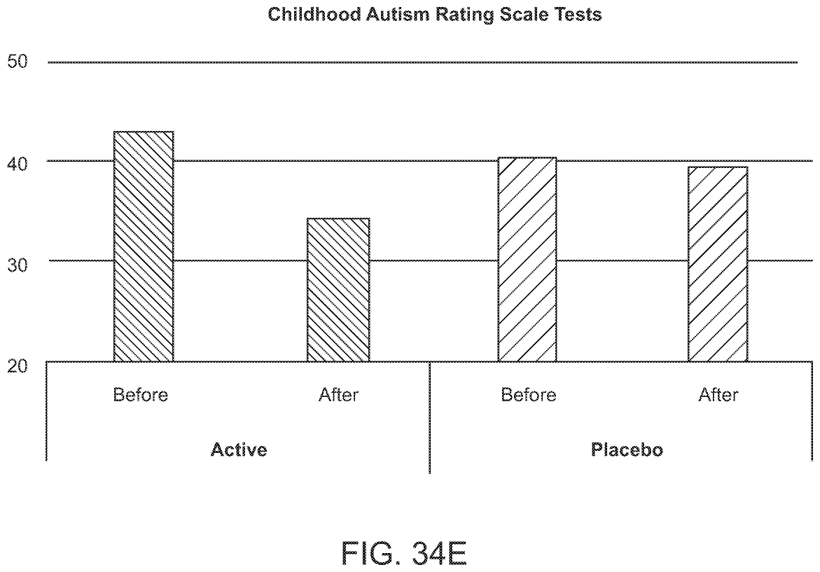

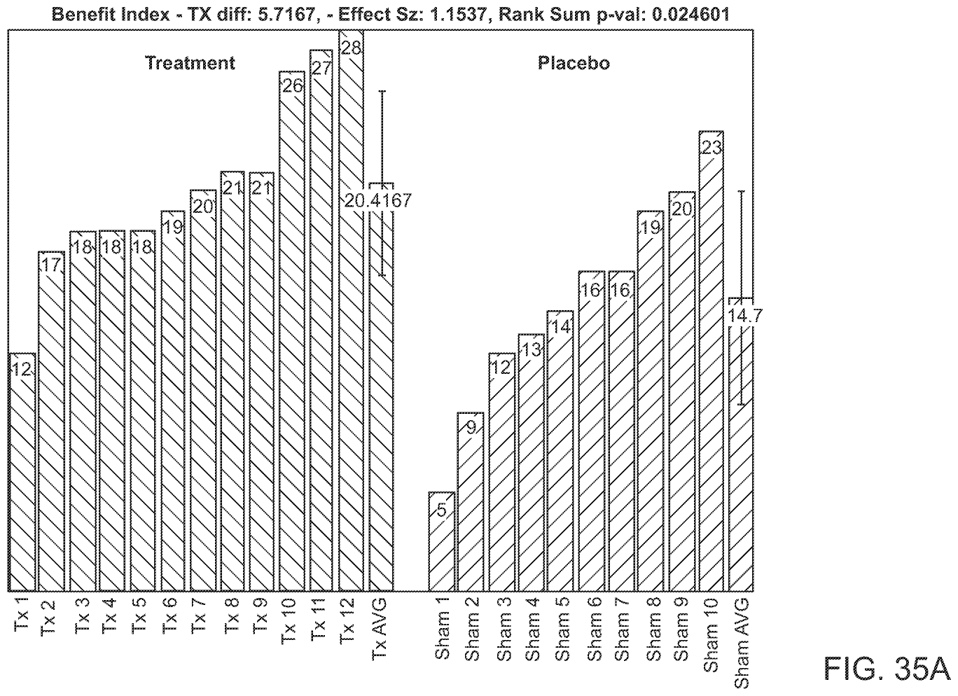

Clinical trials also indicate that systems and methods for photobiomodulation as described herein can produce measurable improvements in EEG data. EEG data was gathered before and after treatment from the frontal, occipital, and temporal areas in consenting subjects. (In some cases, it was not permissible or possible to collect EEG data from a child, for example, due to hair interference or sensory issues, and collected data was meaningless for analysis in some cases such as when a child is jumping around.) The data was analyzed by the Pirogov Institute in Moscow. Changes in the EEG data show a decrease in delta waves and increase in alpha, beta, and theta in a few patients which is associated with better focus, implicit learning and faster language acquisition. Compared to placebo, active stimulation using photobiomodulation systems and methods as described herein presented suppression of the increase in the lower frequency bands (delta) and a further increase in power in the higher frequency bands (alpha, beta,). Normalization of alpha activity can represent normalization of DMN functions and is indicative of increased organization in the cortex including language areas. A summary of the trajectory of the alpha and beta is 13.2-15.36-8.78-7.45-21.8-27.7 (increasing). A summary of the trajectory of the theta wave is 49.08-51.04-58.92-71.6-52.9-61.7 (increasing). Note that ASD is associated with lower theta wave values. Consequently, the measured increase in theta waves and the decrease in delta waves serves as a biomarker for improvement of cognitive function of ASD patients. A summary of the trajectory of the delta wave is 0-31.4-27.8-19.2-27.7-0 (decreasing). FIG. 34A illustrates the decreasing trend of the delta wave for the experimental group (“active”) including six individual subjects. Averaged data and a curve fit are also illustrated. FIG. 34B illustrates delta wave data for eleven subjects in the control group (“placebo”). After completion of the study, an overlay of the active and control groups of the EEG delta wave component is shown in FIG. 34C for data measured over seven sessions. The data indicate a statistically significant decrease in the active group undergoing photobiomodulation therapy. As can be seen, there does not appear to be a consistent trend among the control subjects with no visible decrease in delta wave intensity.

Progress of the subjects in the study was also analyzed through qualitative interviews. Specifically, a researcher conducted weekly interviews with parents regarding their observation of the children. In addition, most of the time when the parent came in the researcher collected notes as well. The table below shows averaged data for experimental and control groups for individual question categories and for aggregated “index” scores as described below:

| QuestionTitles | TxAVGs | TxSTDs | PlaceboAVGs | PlaceboSTDs | Pvals | EffectSizes |
|---|---|---|---|---|---|---|
| Benefit Index | 20.42 | 4.62 | 14.70 | 5.33 | 0.0246 | 1.1537 |
| Eye Contact | 4.42 | 2.11 | 2.30 | 1.49 | 0.0159 | 1.1394 |
| Improvement | | | | | | |
| Hitting Self | 1.08 | 1.38 | 0.30 | 0.48 | 0.1921 | 0.7302 |
| Social Improvement | 3.83 | 2.76 | 2.20 | 1.69 | 0.1602 | 0.6988 |
| Command | 4.75 | 2.05 | 3.50 | 2.32 | 0.2862 | 0.5743 |
| Improvement | | | | | | |
| Anxiety | −0.42 | 1.68 | 0.50 | 1.65 | 0.2375 | 0.5507 |
| Improvement | | | | | | |
| Wakeup | 1.00 | 1.60 | 0.20 | 1.62 | 0.5824 | 0.4981 |
| Improvement | | | | | | |
| Headaches | 0.42 | 0.90 | 0.10 | 0.32 | 0.3767 | 0.4520 |
| Side Effect Index | 4.17 | 3.07 | 3.20 | 2.62 | 0.5493 | 0.3363 |
| Tics | 0.42 | 1.24 | 0.10 | 0.32 | 1.0000 | 0.3355 |
| Meltdowns | 32.33 | 54.17 | 20.60 | 19.49 | 0.6679 | 0.2777 |
| Calmer | 1.58 | 2.19 | 1.10 | 1.10 | 0.9170 | 0.2706 |
| Meltdowns | 0.00 | 1.48 | 0.50 | 2.27 | 0.5003 | 0.2663 |
| Improvement | | | | | | |
| Hyperactivity? | 2.67 | 2.23 | 2.20 | 2.20 | 0.6159 | 0.2105 |
| New Words How | 13.25 | 8.29 | 15.00 | 9.18 | 0.7660 | 0.2011 |
| Many | | | | | | |
| Wakeups | 7.25 | 8.25 | 5.70 | 7.45 | 0.5948 | 0.1962 |
| Speech | 5.83 | 1.59 | 5.60 | 2.59 | 0.7880 | 0.1112 |
| Improvement | | | | | | |
| Eating | 0.83 | 2.37 | 1.00 | 1.33 | 0.8930 | 0.0846 |
| Improvement | | | | | | |

A ‘total improvement score’ was computed by combining the categories that are related to socialization (e.g. eye contact), language (new words), and responsiveness to create the total Benefit Index score. FIG. 34D shows an aggregate comparison of the active and control groups with the active group demonstrating a higher score. The placebo group showed improvement that can reflect a natural improvement or improvement due to a “placebo effect.” FIG. 34E shows an aggregate CARS graphical illustration of the before and after results for both active and control groups of the study with the active group showing a statistically significant decrease in comparison with the control group.

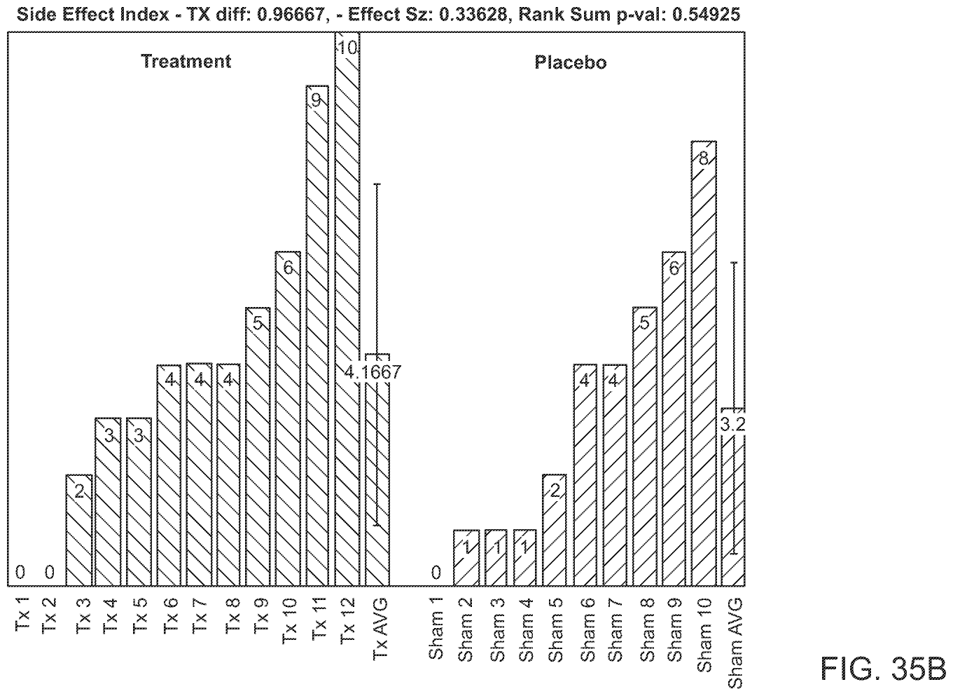

FIG. 35A illustrates values of the benefit index for individual subjects and the average for all subjects including error bars. The results show a statistically significant difference in the Benefit score between the Active and Placebo kids. Separately, the categories that relate to “over-excitement” such as hyperactivity, headaches, wakefulness, and others can be combined to create the Side Effect Index score. FIG. 35B illustrates values of the side effect index for individual subjects and the average for all subjects including error bars. The results also show a non-statistically significant difference in Side Effects between Active and Placebo children.

A further analysis of clinical results can be based on a treatment protocol taking place 2 times a week for up to 60 minutes at a time. During this patient study, the participants first wear the mobile EEG device for approximately 15 minutes, and the EEG signals before treatment are collected. Then the participants wear the tPBM device for up to 15 minutes. Then the participants wear the EEG device again for approximately 15 more minutes. The child can be encouraged to play with toys and interact with the parent (or the experimenter). The children in the active and sham treatment group wear the same devices (the tPBM device won't be turned on for children in the sham group). The total time in the office can be about 40-60 minutes (including playing). An example assessment schedule is shown in the table below

### Assessment Schedule

| | | Twice a | Once a | | |
|---|---|---|---|---|---|
| Visit | Baseline | Week | week | Visit 12 | Visit 24 |
| Demographics | X | | | | |
| Therapeutic | | X | | X | X |
| Treatment/ | | | | | |
| Sham treatment | | | | | |
| CARS2 | X | | | X | X |
| SRS | X | | | | X |
| REELS-3 | X | | | | X |
| EEG | X | X | | X | X |
| Parental Interviews | X | | X | X | X |
| Therapist Interviews | X | | | X | X |

Several Standard tests (scales) can be used for pre-test and post-test, as well as weekly interviews.

### Primary End Point:

1. Childhood Autism Rating Scales, Second Edition (CARS2).
a. Performed by a clinician at the beginning of the trial
b. Performed by a clinician upon the completion of the trial

2. Secondary End POINTS: Social Responsiveness Scales (SRS)
a. Performed by a clinician at the beginning of the trial
b. Performed by a clinician upon the completion of the trial

3. Receptive-Expressive Language Scales, Third Edition (REELS-3)
a. Performed by a clinician at the beginning of the trial
b. Performed by a clinician upon the completion of the trial

4. EXPLORATORY END POINTS: EEG will be collected from each participant.
a. It will be conducted pre- and post-treatment in each session.
b. Mobile FDA-cleared medical grade EEG device.
c. Performed by a research assistant who is conducting each session.

5. Parental interviews.
a. Performed weekly by a research assistant

6. Therapist interviews
a. Performed by a research assistant at the beginning of the trial.
b. Performed by a research assistant at the midpoint of the trial.
c. Performed by a research assistant upon the completion of the trial.

### Endpoints

Primary Safety Measure
Adverse events will be recorded after each therapy session, through
weekly parental interviews, and through bi-weekly therapist interviews

| | |
|---|---|
| Efficacy Measures | Descriptions |

Primary

| | |
|---|---|
| Childhood Autism Rating | (CARS2) is a 15-item rating scale used to assess children |
| Scales, Second Edition | with autism in the following functional areas: |
| (CARS2) | Relating to People |
| | Imitation |
| | Emotional Response |
| | Body Use |
| | Object Use |
| | Adaptation to Change |
| | Visual Response |
| | Listening Response |
| | Taste, Smell and Touch Response and Use |
| | Fear or Nervousness |
| | Verbal Communication |
| | Nonverbal Communication |
| | Activity Level |
| | Level and Consistency of Intellectual Response |
| | General Impressions |
| | The clinician rates the individual on each item, using a 4- |
| | point rating scale, ranging from minimal or no symptoms to |
| | severe symptoms of ASD. Ratings are based on frequency of |
| | the behavior in question, its intensity, peculiarity, and |
| | duration. |
| | The cutoff for ASD diagnosis is 30. The higher the score, the |
| | more severe the condition. Score of 37 and higher indicates |
| | severe autism. Score of 30 to 36.5 indicates mild to moderate |
| | autism. |
| | The CARS-2 is an update of the Childhood Autism Rating |
| | Scale (CARS), an older and widely-used rating scale for |
| | autism. CARS and CARS2 were developed on children and |
| | adults referred to the Division TEACCH programs in North |
| | Carolina. Division TEACCH was created in 1966 as a |
| | pioneering program serving individuals on the autism |
| | spectrum of all ages throughout the state of North Carolina. |
| | The original CARS was developed on 1606 children. For the |
| | CARS2-ST, a verification sample of 1034 was obtained. To |
| | develop the CARS2-HF, a sample of 994 was obtained. All |
| | participants were clinically evaluated through Division |
| | TEACCH. |
| | This scale has been validated: |
| | Moulton et. al in 2019 validated a study of 282 children and |
| | concluded the continuing relevance of CARS2 in ASD |
| | assessment. |
| | https://www.ncbi.nlm.nih.gov/pmc/articles/PMC5392181/ |
| | In 2019, Moon et. al. conducted a large meta-analysis with |
| | 24 studies and 4,433 participants. The sensitivity of the test |
| | was acceptable but not the specificity, suggesting that |
| | CARS2 should be used in conjunction with other scales. |
| | https://pubmed.ncbi.nlm.nih.gov/30977125/ |

Secondary

| | |
|---|---|
| Social Responsiveness | The SRS-2 provides a continuous measure of social ability |
| Scales (SRS) | (from impaired to above average) for individuals with 2-18 |
| | years old. High scores are associated with more severe social |
| | impairments. The authors of the questionnaire suggest that |
| | because scores along a continuum can be obtained, the SRS-2 |
| | can help clinicians identify and understand the group of |
| | individuals with ASD with milder impairments as well as |
| | individuals with non-ASD conditions who also show social |
| | impairments. |
| | The SRS-2 has been widely adopted in genetic research on |
| | ASD because it can measure social ability in all family |
| | members (those with an ASD diagnosis and those without). |
| | SRS scale is administered in the format of parent or teacher |
| | questionnaire (5 sub-domains, 65 items on a 4-point Likert |
| | scale). |
| | Normative data was collected through 5 studies: 3 |
| | epidemiological studies of twins studied for other purposes |
| | (only one twin was used for the norm sample) and 2 studies |
| | specifically for SRS norm data. The total norm sample was |
| | approximately 1600 children. |
| | The SRS-2 has been widely adopted in autism research, |
| | especially studies of the genetics of autism. |
| Receptive-Expressive | The Receptive-Expressive Emergent Language Test-Fourth |
| Language Scales, Third | Edition (REEL-4) was designed to help identify toddlers |
| Edition (REELS-3) | who have language impairments or who have other |
| | disabilities that affect language development. The REEL-4 |
| | has two subtests that make up the Language Ability |
| | composite, Receptive Language and Expressive Language, |
| | as well as a supplementary Vocabulary Inventory test, |
| | composed of the Nouns and Expanded subtests. Results are |
| | obtained from a caregiver interview. |
| | The REEL-4 is based on a contemporary linguistic model. It |
| | includes current studies related to normative base, reliability, |
| | and validity. The normative sample includes 1,019 infants |
| | and toddlers from around the nation. The demographic |
| | characteristics of the sample were matched to U.S. child |
| | population for the year 2019 reported in ProQuest Statistical |
| | Abstract of the United States 2018. The normative sample |
| | was stratified to on the basis of gender, race, Hispanic status, |
| | and geographic region. Standard scores, percentile ranks, and |
| | age equivalents are provided. |
| | The average reliability coefficients for all test scores are high |
| | (exceeding .90). Test-retest studies show the REEL-4 is |
| | stable over time. |
| | Studies of the test’s diagnostic accuracy (sensitivity, |
| | specificity, and ROC/AUC statistics) support its use for |
| | differentiating children with language impairment, low- |
| | functioning autism, and developmental delay from children |
| | with no exceptionalities. Validity data are reported as well, |
| | documenting the test’s relationship to the Developmental |
| | Assessment of Young Children-Second Edition, Preschool |
| | Language Scales-Fifth Edition, Receptive-Expressive |
| | Emergent Language Test-Third Edition, and Test of Early |
| | Communication and Emerging Language. |

Exploratory

| | |
|---|---|
| EEG | EEG data could potentially reveal statistically changes in the |
| | distribution of brainwaves as a result of the treatment. Delta |
| | waves in the wakeful state could be a sign of brain |
| | inflammation (e.g., Frohlich et al, 2021). High intensity of |
| | Delta waves and their reduction during the course of the |
| | study could be indicative of the reduction of brain |
| | inflammation. Medical grade EEG (at least 16 channel) will |
| | be collected during each session to measure the intensity of |
| | different brainwaves in active and sham conditions. |
| Parental Interview | Structured parental interviews will be used to monitor |
| | positive events (e.g., new words learned, increased focus, |
| | increased eye-contact) and monitor side-effects (e.g., loss of |
| | sleep, hyperactivity) throughout the course of the study |
| Therapist’s interview | Structured therapist’s interview with questions pertaining to |
| | children’s ability to focus, ability to sit calmly throughout |
| | the session, respond to the therapist, indicate their needs will |
| | be conducted before and after the course of treatment in the |
| | active and sham arms. |

### Primary Endpoint: Childhood Autism Rating Scale (CARS2)

### Secondary Endpoints:

Social Responsiveness Scales (SRS)
Receptive-Expressive Language Scales, Third Edition (REELS-3)
EEG

### Exploratory Endpoints:

Synergy of tPBM treatment and ABA Therapy
Parental interviews.
Therapist interviews

Statistics: There are no universally accepted clinical outcome measures developed for measuring changes in core symptoms in ASD, based on interventions. A recently proposed clinical efficiency benchmark is a 4-4.5 decrease in CARS 2, based on a recent article by Jurek et. al. 2021, who conducted a panel of 5 experts including pediatric and adult psychiatrists who work with patients in Europe and India. Their proposal should be taken with caution, because they do not work with a diverse population, similar to the population in the United States. We propose a reduction of 3 points in CARS, as it is a 10% reduction—based on 30 points being a cutoff Score for ASD. Clinically, 10% reduction of CARS score may mean moving from SEVERE to Moderate or from Moderate to Mild subgroup of the spectrum, which might mean more independent functioning (e.g., improved signaling of their needs, improved focus, responsiveness to language and therapy) and improved quality of life for children and their caregivers.

A sample size of 60 subjects, 30 in each arm is sufficient to detect a clinically important difference of 3 points between treatment groups in reducing symptoms of autism as measured by the CARSII assessment assuming a between treatment standard deviation of 8.02 using a two-tailed t-test of difference between means with 80% power and a 5% level of significance. to reach significance in the primary end point. If we reach statistical and clinical significance with 60 participants, we plan to continue recruiting up to 150 participants to measure the effectiveness in the secondary and exploratory end points.

Analysis Population: Data analysis will be conducted on the intent to treat (ITT) population which includes all subjects that were consented and randomized to either the JelikaLite device or the sham device. Additional analysis will be conducted on a per protocol population which is defined as all participants who have participated in the trial.

Effectiveness Analysis: The following analyses are conducted:
1. Primary analysis (based on 60 participants):
   a. Before and After treatment change in CARS scores in Active and Sham groups,
   b. Analysis of the number of subjects that achieved clinically meaningful difference, based on CARS
2. Statistical significance with CARS: based on power calculation based on CARS the following secondary analysis on the full data set at the end of the full study (with 150 participants).
   a. SRS and REELS: Before and After treatment changes in the scores in the Active and Sham groups.
   b. EEG data (Before and After treatment redistribution of the brainwaves in Active and Sham groups)
3. Exploratory end points:
   a. Correlation between the effectiveness of tPBM and Number of ABA hours received (using CARS).
   b. Analysis of Qualitative data such as parental interviews and therapists' interviews.

The primary analysis of efficacy includes a comparison of change in mean CARSII scores between Baseline and 12 for the actively treated versus sham arm. The primary analysis will be performed on the ITT population.

A model for repeated measures fitted by restricted maximum likelihood method will be used for the primary analysis. This model takes into account the presence of missing data and yields valid estimates under the assumption of data missing at random (MAR).

Fixed effects will further include treatment, visit, treatment by visit interaction, baseline CARSII score and baseline CARSII by visit interaction. A general (co)variance structure with unconstrained correlations and variances will be used to model the within-subject errors. If this analysis fails to converge, alternative variance-covariance structures will be considered. More specifically, the same mean model will be fitted with following variance-covariance structure (in this order):
An antedependence correlation structure
A heterogeneous Toeplitz correlation with unconstrained variances
A heterogeneous compound symmetry structure
   The Kenward-Roger approximation can be used to estimate denominator degrees of freedom.

The Secondary Analysis of Efficacy: In the first part of the secondary analysis of efficacy, a comparison of change in mean SRS and REEL scores is calculated between Baseline and Week 12 for the actively treated versus sham arm. The primary analysis will be performed on the ITT population. A model for repeated measures fitted by restricted maximum likelihood method will be used for the primary analysis. This model takes into account the presence of missing data and yields valid estimates under the assumption of data missing at random (MAR). Fixed effects can further include treatment, visit, treatment by visit interaction, baseline SRS and REEL score and baseline SRS and REEL scores by visit interaction. A general (co)variance structure with unconstrained correlations and variances can be used to model the within-subject errors. If this analysis fails to converge, alternative variance-covariance structures will be considered.

In a second part of the secondary analysis of efficacy, linear regression analysis can be used to analyze the change in EEG waves distribution throughout the measurement. Pearson correlation of the distribution of brainwaves with time can be computed.

Exploratory Endpoint Analysis: CARS: Synergy of tPBM treatment and ABA therapy: The model will include ABA treatment used in the randomization as covariate (3 levels: 0 hours of ABA, <=10 hours of ABA and >10 hours of ABA.). This analysis will be conducted if there is a statistically significant correlation of the before and after treatment change in CARS scores and the number of ABA hours each participant in receiving. Parental Interviews: Non-parametric statistics (e.g., Wilcoxon Signed Rank test). Therapist Interviews: Non-parametric statistics (e.g., Wilcoxon Signed Rank test).

Additional confounding variables for Post-Trial Analysis:
Age
Skin color
Length, color and thickness of hair (we will encourage all participants to cut hair for the duration of the trial).
Severity of condition
Gender
Other concomitant treatments
# of languages spoken in the household
Changes in amount of therapy during the trial
Non-psychotropic medication

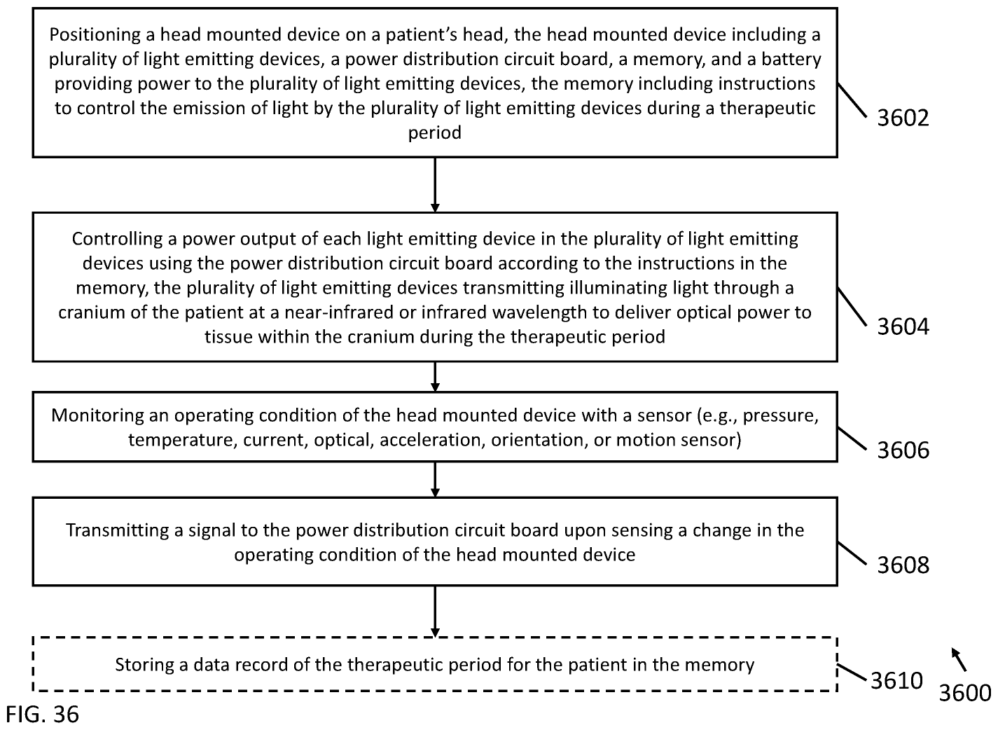

FIG. 36 illustrates a method 3600 for therapeutic photobiomodulation for treatment of diseases or disorders in accordance with some embodiments described herein. The method 3600 includes positioning a head mounted device 5000 on a patient's head (step 3602). The head mounted device 5000 includes a plurality of light emitting devices 5036, a power distribution circuit board 5040, 5050, a memory 5042, and a battery 5060 providing power to the plurality of light emitting devices 5036. The memory 5042 includes instructions to control the emission of light by the plurality of light emitting devices 5036 during a therapeutic period. The method 3600 includes controlling a power output of each light emitting device 5036 in the plurality of light emitting devices using the power distribution circuit board 5040, 5050 according to the instructions in the memory 5042 (step 3604). The plurality of light emitting devices transmits illuminating light through a cranium of the patient at a near-infrared or infrared wavelength to deliver optical power to tissue within the cranium during the therapeutic period. The method 3600 includes a step of monitoring an operating condition of the head mounted device 5000 with a sensor (step 3604). The sensor can be a pressure, temperature, current, optical, or motion sensor in various embodiments. The sensor can measure acceleration or orientation in some embodiments similar to the accelerometer 5045 employed by the patient detection module 5046. Monitoring the operating condition can include detecting adverse events such as monitoring whether the head mounted device 5000 has been removed (advertently or inadvertently) from the patient's head, monitoring temperature to determine if the device is overheating, or monitoring optical power or current to determine whether the device is transmitting too great of an intensity of optical power. The method 3600 can also include transmitting a signal to the power distribution circuit board 5040, 5050 upon sensing a change in the operation condition of the head mounted device 5000 (step 3608). For example, a signal can be sent to the power distribution circuit board 5040, 5050 to stop power upon sensing a parameter that indicates an adverse operating condition. Conversely, a signal can be sent to the power distribution circuit board 5040, 5050 that enables power distribution to the LEDs if the sensor detects that the operating condition is safe (i.e., no errors or warnings). The method 3600 also includes an optional step of storing a data record of the therapeutic period for the patient in the memory 5042 (step 3610). In such an embodiment, the memory 5042 can be non-volatile (e.g., EEPROM or solid-state storage) or the memory 5042 can be volatile memory such as any of the various forms of random access memory (RAM).

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described can include a particular feature, structure, or characteristic, but not every implementation necessarily includes the particular feature, structure, or characteristic. Further, repeated use of the phrase “in one implementation” does not necessarily refer to the same implementation, although it can.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than the order shown in the illustrative flowcharts.

What is claimed is:

1. A photobiomodulation neuro-therapy device comprising:
a portable head mounted device that is sized to be positioned on a patient's head, the portable head mounted device including a plurality of light emitting devices, a processor, a memory and a battery providing power to the portable head mounted device, each of the light emitting devices being operable in response to control signals to control transmission of transcranial illuminating light into the patient having a near infrared wavelength delivered during a therapeutic period wherein the processor executes instructions stored in the memory to control the emission of light by the light emitting devices during the therapeutic period; and
a power control circuit on the portable head mounted device, the power control circuit being connected to each of the light emitting devices to transmit the control signals that separately control emission of light from each of the light emitting devices.

2. The device of claim 1 further comprising a transducer device that delivers an auditory signal to the patient during the therapeutic period.

3. The device of claim 1 wherein the processor controls a delivery of an auditory signal to the patient with headphones on the portable head mounted device and wherein the memory records the power of the transmitted light delivered to the patient during the therapeutic period.

4. The device of claim 1 further comprising a communication circuit including a transceiver on the portable head mounted device to receive a wireless control signal to control an operation of the portable head mounted device.

5. The device of claim 1 wherein the light emitting device further comprises a first light emitter that illuminates the patient at a first wavelength and a second light emitter that illuminates the patient at a second wavelength different than the first wavelength.

6. The device of claim 1 wherein the light emitting device further comprises a plurality of panels that illuminate the patient from a plurality of different angles, each panel having one or more light emitting diodes (LEDs).

7. The device of claim 1 further comprising a sensor that measures an operating condition of the device or a physiologic response of the patient to the illuminating light and wherein the processor, in response to a sensed value from the sensor, controls an operation of the portable head mounted device.

8. The device of claim 1 wherein the processor is configured to perform at least one of
execute a machine learning operation to determine an operating parameter of the portable head mounted device, or
communicate with an external computing device that performs a machine learning operation to determine an operating parameter of the portable head mounted device.

9. The device of claim 8 wherein the machine learning operation comprises an iterative computational sequence executed on at least one of the processor or the external computing device.

10. The device of claim 1 wherein the portable head mounted device has a size, shape and weight to be worn by a child, and wherein the processor is programmed to treat a neurological condition that comprises autism.

11. The device of claim 1 further comprising an EEG sensor that measures EEG signals of the patient with a plurality of EEG electrodes attached to the head of the patient.

12. The device of claim 6 wherein each panel in the plurality of panels comprises an LED circuit board on which the at least one light emitting diode is mounted, each LED circuit board being connected to a power control circuit mounted on the portable head mounted device.

13. The device of claim 12 wherein the battery is connected to the power control circuit mounted on a controller circuit board.

14. The device of claim 4 wherein the communication circuit communicates with the external computing device by a cable, a wireless transmission or a combination thereof.

15. The device of claim 14 wherein the external computing device comprises a tablet display device having a touchscreen display that is operative in response to a plurality of touch gestures made by a user on the surface of the touchscreen display, the tablet display device including a processor programmed with one or more software modules to control operations of the tablet display device and the portable head mounted device.

16. The device of claim 1 wherein the power control circuit operates under closed loop control to control power distribution to each of the light emitting device.

17. The device of claim 7 wherein the sensor detects a movement of the head mounted device relative to the head of the patient, the sensor transmitting a signal to shut off the light emitting devices.

18. The device of claim 1 wherein the power control circuit operates in response to programmed instructions such that the processor executes a sequence of steps to illuminate different regions of brain tissue of the patient with selected levels of light.

19. The device of claim 18 wherein the selected levels of light comprise a plurality of presets such that a user can select at least one preset that includes a time duration, a total area of a cranium of the patient to be illuminated and a total amount of light to be delivered onto the total area of the cranium of the patient during a therapeutic session.

20. The device of claim 18 wherein the selected levels of light are manually selected by a user with a user interface.

21. The device of claim 1 wherein an external computing device comprises a user interface that controls an operation of the portable head mounted device.

22. The device of claim 21 wherein the portable head mounted device is connected to the external computing device with a cable.

23. The device of claim 21 wherein the portable head mounted device communicates with the external computing device with a wireless connection wherein said communication includes illumination parameters and an illumination period.

24. The device of claim 21 wherein the user interface comprises a graphical user interface operable on a display of the external computing device.

25. The device of claim 24 wherein the external computing device comprises a tablet display device.

26. The device of claim 25 wherein the tablet display device comprises a touchscreen display.

27. The device of claim 26 wherein the graphical user interface is operable on the touchscreen display that is responsive to a plurality of touch gestures whereby a user can control one or more operating parameters of the portable head mounted device.

28. The device of claim 1 wherein the power control circuit comprises a first power control circuit board and further comprising a second power control circuit board, the first power control circuit board being electrically connected to a first plurality of the light emitting devices and the second power control circuit board being connected to second plurality of the light emitting devices, the first plurality of light emitting devices being controlled separately from the second plurality of light emitting devices.
